(19) United States
(12) Patent Application Publication
(10) Pub. No.: US 2013/0323739 A1
(43) Pub. Date: Dec. 5, 2013

(54) METHOD FOR DETERMINING RECURRENCE OR STABLE DISEASE AFTER TREATMENT FOR PROSTATE CANCER

(71) Applicants: Robert E. Klem, Rancho Santa Fe, CA (US); Jonathan E. McDermed, Carlsbad, CA (US); Russ Saunders, Carlsbad, CA (US); Edward Jablonski, Escondido, CA (US); Thomas Adams, Rancho Santa Fe, CA (US); Mark J. Samo, Encinitas, CA (US)

(72) Inventors: Robert E. Klem, Rancho Santa Fe, CA (US); Jonathan E. McDermed, Carlsbad, CA (US); Russ Saunders, Carlsbad, CA (US); Edward Jablonski, Escondido, CA (US); Thomas Adams, Rancho Santa Fe, CA (US); Mark J. Samo, Encinitas, CA (US)

(73) Assignee: Iris International, Inc., Chatsworth, CA (US)

(21) Appl. No.: 13/844,639

(22) Filed: Mar. 15, 2013

Related U.S. Application Data
(60) Provisional application No. 61/655,972, filed on Jun. 5, 2012.

(51) Int. Cl. G01N 33/50; G01N 33/68

(52) U.S. Cl. CPC G01N 33/5091; G01N 33/689
USPC 435/6.12

(57) ABSTRACT

This invention describes compositions and methods for use in PSA assays having low functional sensitivity which are useful, for example, in the detection of early stage recurrence of prostate disease following treatment and in the determination of whether patients have prostate cancer recurrence or stable disease.

### RELATED APPLICATIONS

The present application claims benefit of U.S. provisional application Ser. No. 61/655,972 filed on Jun. 5, 2012. This application also claims priority to U.S. application Ser. No. 13/489,430, which is a continuation-in-part of U.S. Ser. No. 12/378,965 filed on Feb. 19, 2009, which claims benefit of U.S. provisional application Ser. No. 61/066,732, filed on Feb. 21, 2008, U.S. Ser. No. 61/030,718, filed on Feb. 22, 2008, and U.S. Ser. No. 61/030,462, filed on Feb. 21, 2008. The contents of each of these applications are incorporated by reference herein in their entirety.

### BACKGROUND AND INTRODUCTION TO THE INVENTION

The following includes information that may be useful in understanding the present invention. It is not an admission that any of the information provided herein is prior art, or relevant, to the presently described or claimed inventions, or that any publication or document that is specifically or implicitly referenced is prior art.

Prostate cancer is the most prevalent male malignancy in the United States with nearly a quarter million new cases diagnosed each year. Age at diagnosis has decreased with a concomitant increase in the number of men diagnosed with early-stage or clinically localized disease. Jemal A., Tiwari R. C., Murray T., et al: Cancer statistics, 2004. CA Cancer J. Clin. 2004: 54: 8-29; Moul J. W., Wu H., Sun L., et al: Epidemiology of radical prostatectomy for localized prostate cancer in the era of prostate-specific antigen: An overview of the Department of Defense Center for Prostate Disease Research national database. Surgery 2002; 132: 213-219.

Radical prostatectomy (RP) is an increasingly common treatment for localized prostate cancer perhaps due to wider adoption of robotic-assisted laparoscopic technology. While surgery can result in long-term cancer control more men are living longer with prostate cancer, leading to a proportionately larger number of men at risk for cancer recurrence heralded by a rising PSA that exceeds a defined cutpoint, typically 0.2-0.4 ng/mL (200-400 pg/mL). Pound, C. R., Partin, A. W., Eisenberger, M. A., et al: Natural history of progression after PSA elevation following radical prostatectomy. JAMA 1999; 281: 1591-1597; Liu, L., Coker, A. L., Du, X. L., et al: Long-term survival after radical prostatectomy compared to other treatments in older men with local/regional prostate cancer. J. Surg. Oncol. 2008; 97: 583-591; Gretzer, M. B., Trock, B. J., Han, M., et al: A critical analysis of the interpretation of biochemical failure in surgically treated patients using the American Society for Therapeutic Radiation and Oncology criteria. J. Urol. 2002; 168: 1419-1422; Roehl, K. A., Han, M., Ramos, C. G., et al: Cancer progression and survival rates following anatomical radical retropubic prostatectomy in 3,478 consecutive patients: long-term results. J. Urol. 2004; 172: 910-914.

In addition to risk factors such as family history of cancer, smoking status, age, and race, the initial detection of prostate cancer is generally based upon findings of increased circulating concentrations of a protein called Prostate-Specific Antigen (PSA), a neutral serine protease produced by normal, benign and malignant prostatic epithelial cells. PSA produced by prostatic cells is present in both free and complexed forms in seminal fluid, serum, plasma, and urine and can be measured in those fluids. Simultaneous measurement of the free and complexed forms is called “total PSA” measurement and may be referred to correctly as either “tPSA” or “PSA.” The concentration of PSA in blood increases in various prostate diseases, particularly in prostate cancer, and this increased concentration is reflected in serum measurements of PSA. Valsanen et al., Prostate Cancer and Prostatic Disease 2:91-97 (1999). Thus, for the past two decades, assays such as conventional immunoassays for serum PSA have been used in the initial detection of prostate cancer. Yu et al., J. Urology 157:913-918 (1997).

Biochemical recurrence (BCR) is conservatively estimated to affect around 50,000 men each year. Moul J W: The evolving definition of advanced prostate cancer. Rev. Urol. 6 (Suppl. 8): S10-S17, 2004. As a result, both physicians and the men treated by RP are interested in estimating the risk of clinical recurrence. Post-RP risk stratification approaches are based on a number of factors, but rely heavily on pathologic findings, such as Gleason score, percentage of grade 4-5 disease, surgical margin involvement, extracapsular extension (also referred to as capsular extension), seminal vesicle invasion and lymph node involvement. Multivariate models (nomograms) that incorporate these factors are freely available on the Internet to assist in post-RP risk assessment. http://www.nomogram.org/Prostate/pros_calc.php.

However, these models predict the likelihood of BCR and are not suitably generalizable for identifying men at high or low risk of clinical recurrence, either locally or systemically. Shariat, S. F., Kattan, M. W., Vickers, A. J., et al: Critical review of prostate cancer predictive tools. Future Oncology 2009; 5: 1555-1584.

The serum PSA nadir value following RP can be one factor in predicting BCR since undetectable levels <0.01 ng/mL (<10 pg/mL) are associated with better prognosis when BCR is used as the endpoint. See Witherspoon, L. R., Lapeyrolerie, T.: Sensitive prostate specific antigen measurements identify men with long disease-free intervals and differentiate aggressive from indolent cancer recurrences within two years after radical prostatectomy. J. Urol. 1997; 157: 1322-1328; Doherty, A. P., Bower, J., Smith, G. L., et al: Undetectable ultrasensitive PSA after radical prostatectomy for prostate cancer predicts relapse-free survival. Br. J. Cancer 1999; 83: 1432-1436; Hong, S. K., Park, H. Z., Lee, W. K., et al: Prognostic significance of undetectable ultrasensitive prostate-specific antigen nadir after radical prostatectomy. Urology 2010; 76: 723-727; Shen, S., Lepor, H., Yaffee, R., et al: Ultrasensitive serum prostate specific antigen nadir accurately predicts the risk of early relapse after radical prostatectomy. J. Urol. 2005; 173: 777-80.

However, most men have serum PSA concentrations following RP that are undetectable with these assays and their poor sensitivity cannot distinguish between men that are at increased risk of recurrence and those that may be at reduced risk of recurrence. Shimizu, F., Matsuyama, Y., Tominaga, T., et al: Inadequacy of prostate-specific antigen doubling time estimates calculated using an ultrasensitive assay of prostate-specific antigen for biochemical failure after radical prostatectomy. Urol. Int. 2007; 79: 356-360; Taylor, J. A., Koff, S. G., Dauser, D. A., et al. The relationship of ultrasensitive measurements of prostate-specific antigen levels to prostate cancer recurrence after radical prostatectomy. BJU Int. 2006; 98: 540-543.

The inability to differentiate clinical recurrence risk based on these assays is unfortunate for several reasons. From a healthcare management perspective, the intensity of serum PSA and urologic follow-up could be reduced in men at low risk for clinical recurrence. In addition, empiric use of adjuvant therapies could be reduced or eliminated, which could also reduce healthcare costs and costs of unneeded therapy. From a patient perspective, quality of life would improve with the avoidance of unneeded radiotherapy or hormonal therapy, safer treatment of hypogonadism, and the unquantifiable yet important alleviation of psychological stress.

Generally, if increased serum PSA concentrations are observed in a patient, a prostate biopsy is performed to confirm the presence of cancer and to characterize the cancer pathology. Once prostate cancer is confirmed, approximately two-thirds of patients are treated with radical prostatectomy (RP, the complete surgical removal of the prostate), or radiation, hormonal, or chemotherapies by a variety of methods. However, up to 40% of those treated patients may undergo disease recurrence. See Moul, J. Urology 163:1632-1642 (2000). Recurrence of prostate cancer is associated with a poor prognosis for survival. However, prognosis can be improved if the recurrence is detected at an early stage so that appropriate management methods including salvage and/or adjuvant treatments may be initiated. Unfortunately, existing methods for evaluating the likelihood of recurrence are insufficient for early detection. Clinicopathological observations taken prior to, or at the time of RP such as cancer stage, Gleason score, age at diagnosis, surgical margin involvement (presence of cancer at the surgical margin), local tissue invasion of the cancer, prostate capsule invasion of the cancer, seminal vesicle invasion of the cancer, bladder neck invasion of the cancer, lymph node invasion of the cancer, and total tumor volume are somewhat informative in assessing the likelihood of disease recurrence but are not always predictive and cannot be used to identify the exact time of a recurrence. Biopsy or imaging methods of various types can be used to confirm disease recurrence but these methods suffer from poor sensitivity. Generally, by the time a biopsy or imaging study detects new tumors, the recurrence is at a late stage when prognosis is especially poor. Thus, these methods are insufficient for early detection and aggressive treatment based thereon.

### BRIEF SUMMARY OF THE INVENTION

Accordingly, to address the insufficiencies of basing disease recurrence on clinicopathological findings and biopsy or imaging studies, in one aspect this invention relates to determining prostate cancer recurrence based on findings of increasing serum PSA concentrations in the patient following treatment, in combination with clinicopathological findings. In some aspects this invention is useful for determining whether a patent has recurrence or a likelihood of stable disease following treatment for prostate cancer which results in [PSA] levels less than 100 ng/mL. For example, following a radical prostatectomy or androgen deprivation treatment where little or no residual, PSA-secreting prostate tissue remains and sufficient time has passed for the physiological clearance of pre-operative levels of PSA, the serum concentration of PSA falls to a nadir. If the serum PSA concentrations begin to rise at a specified rate after the nadir point, a disease recurrence may be indicated.

In “biochemical recurrence” (BCR) observed with other assays where the determination of “recurrence” reflects only an increase in circulating levels of PSA rather than findings of new local or distant tumors. Biochemical recurrence of PSA has been a standard of care in medical management of prostate cancer following treatment such as RP.

Various thresholds have been published to establish the point at which biochemical recurrence is thought to occur. Cookson, M. S., et al. J. Urology 177:540-545 (2007). Typically, a value of 200 pg/mL (0.2 ng/ml) following the nadir of PSA is utilized to define the point of biochemical recurrence. Id. Conventional assays for PSA have detection limits in the range of 100 pg/ml with functional sensitivities possibly higher. The mean detection time for biochemical recurrence using a conventional PSA assay with a detection limit of 100 pg/mL is over 38.4 months. Vassilikos et al., Clinical Biochemistry 33(2): 115-123 (2000).

Aspects and embodiments of the instant disclosure are based on the surprising and unexpected results demonstrating a synergistic potentiation in the predictive value and the sensitivity limit for a method of determining the risk of prostate cancer recurrence in a subject following treatment for prostate disease using a combination of PSA recurrence probability index and pathological findings. In one aspect the determination can be made based on measurements of [PSA] in samples obtained from a patient within 18 months of treatment for prostate cancer resulting in [PSA] of <100 pg/mL, such as radical prostatectomy or androgen deprivation. This novel method enables, for the first time, a highly predictive and highly sensitive assay for assessing recurrence risk in subjects having a post-treatment level of PSA of <100 pg/mL, which were previously below the limit of conventional assay detection.

In one aspect, this invention relates to a method of determining the combined risk of prostate cancer recurrence in a subject following treatment for prostate cancer resulting in a post-treatment level of PSA of <100 pg/mL, comprising: a) determining at least one pathological finding (PF) in the subject following treatment wherein the pathological finding is selected from: a Gleason score of ≧7, a positive surgical margin, seminal vesicle invasion, extracapsular extension (i.e, capsular extension or ECE); b) obtaining measurement of the PSA levels in two or more samples obtained from the patient within 18 months after treatment for prostate cancer, wherein the PSA assay for measuring the PSA levels has a functional sensitivity less than 2.0 pg/mL, and the PSA levels from two or more samples are used to determine a [PSA] time value, where the [PSA] time value does not exceed a PSA recurrence probability index and the combined recurrence risk of the subject is classified as PF+/PSA−; and c) whereupon adjuvant treatment is not administered based on the [PSA]-time value and PSA recurrence probability index (RPI).

In another aspect, this invention relates to a method of determining the combined risk of prostate cancer recurrence in a subject following treatment for prostate cancer resulting in a post-treatment level of PSA of <100 pg/mL, comprising: a) determining at least one pathological finding (PF) in the subject following treatment wherein the pathological finding is selected from, for example, a Gleason score of ≧7, a positive surgical margin, seminal vesicle invasion, extracapsular extension (i.e, capsular extension or ECE); b) obtaining measurement of the PSA levels in two or more samples obtained from the patient within 18 months after treatment for prostate cancer, where the PSA assay for measuring the PSA levels has a functional sensitivity less than 2.0 pg/mL, and the PSA levels from two or more samples are used to determine a [PSA]-time value, where the [PSA]-time value exceeds a PSA-RPI and the combined recurrence risk of the subject is classified as PF+/PSA+; and c) whereupon adjuvant treatment is administered based on the combined risk classification using the [PSA]-time value and PSA-RPI in combination with the pathological findings.

In another aspect, this invention relates to a method of determining the combined risk of prostate cancer recurrence in a subject following treatment for prostate cancer resulting in a post-treatment level of PSA of <100 pg/mL, comprising: a) determining at least one pathological finding (PF) in the subject following treatment wherein the pathological finding is selected from: a Gleason score of less than 7, a negative surgical margin, a negative seminal vesicle invasion, a negative extracapsular extension (i.e, capsular extension or ECE); b) obtaining measurement of the PSA levels in two or more samples obtained from the patient within 18 months after treatment for prostate cancer, wherein the PSA assay for measuring the PSA levels has a functional sensitivity less than 2.0 pg/mL, and the PSA levels from the two or more samples are used to determine a [PSA]-time value, wherein the [PSA] time value does not exceed a PSA RPI and the combined recurrent risk of the subject is classified as PF−/PSA−; and c) whereupon adjuvant treatment is not administered based on the combined risk classification using the [PSA]-time value and PSA-RPI in combination with the pathological findings.

In yet another aspect, this invention relates to a method of determining the combined risk of prostate cancer recurrence in a subject following treatment for prostate cancer resulting in a post-treatment level of PSA of <100 pg/mL, comprising: a) determining at least one pathological finding (PF) in the subject following treatment wherein the pathological finding is selected from: a Gleason score of less than 7, a negative surgical margin, a negative seminal vesicle invasion, a negative extracapsular extension (i.e, capsular extension or ECE); b) obtaining measurement of the PSA levels in two or more samples obtained from the patient within 18 months after treatment for prostate cancer, wherein the PSA assay for measuring the PSA levels has a limit of quantitation functional sensitivity less than 2.0 pg/mL, and the PSA levels from the two or more samples are used to determine a [PSA]-time value, wherein the [PSA]-time value exceeds a PSA RPI of 2.0 pg/mL/month and the combined recurrent risk of the subject is classified as PF−/PSA+; and c) whereupon adjuvant treatment is administered based on the classification using the [PSA]-time value and PSA-RPI.

In one embodiment, the likelihood of recurrence is greater for a subject who is classified as PSA+ than for a subject who is classified as PSA− and PF+.

In one embodiment, the PF is seminal vesicle invasion. In one embodiment, the likelihood of recurrence is greater for a subject who is classified as PSA+ than a subject who is classified as PSA− and seminal vesicle positive. In one embodiment, the likelihood of recurrence is greater than for a subject who is classified as PSA+ than a subject who is classified as PSA− and seminal vesicle negative.

As used herein, the risk in recurrence (or the chance of having a stable disease) may be expressed in terms of odds ratios. For example, when a subject presents with a Gleason score of 7 or greater, and the subject is determined to have a PSA-time values that exceeds the PSA-RPI threshold, the subject then is determined to have about a 8.31 fold higher risk of recurrence than by Gleason score alone. Similarly, if a subject presents with a negative seminal vesicle invasion, and the subject is determined to have PSA-time values that exceeds the PSA-RPI threshold, the subject then is determined to have about a 15.7 fold greater risk of recurrence than by pathological finding alone.

In other embodiments, the increased likelihood of recurrence is, for example, is about at least up to 9.49 times greater for a patient who has a PSA time value exceeding the PSA RPI and a positive pathological finding than for a patient who has a PSA time value which does not exceed the PSA RPI and negative pathological finding. In some embodiments, the increased likelihood of stable disease is, for example, at least up to about 54 times greater for a patient who has a PSA time value not exceeding the PSA RPI index and a negative pathological finding than for a patient who has a PSA time value which exceeding the PSA RPI and a positive pathological finding.

For example, in other embodiments using Gleason score as the clinical observation, and in other embodiments using clinical observations such as surgical margins, capsular extension and/or seminal vesicle invasion, the increased risk of recurrence or likelihood of stable disease is about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, about 12.0, about 12.1, about 12.2, about 12.3, about 12.4, about 12.5, about 12.6, about 12.7, about 12.8, about 12.9, about 13.0, about 13.1, about 13.2, about 13.3, about 13.4, about 13.5, about 13.6, about 13.7, about 13.8, about 13.9, about 14.0, about 14.1, about 14.2, about 14.3, about 14.4, about 14.5, about 14.6, about 14.7, about 14.8, about 14.8, about 14.9, about 15.0, about 15.1, about 15.2, about 15.3, about 15.4, about 15.5, about 15.6, about 15.7, about 15.8, about 15.9, about 16, about 16.1, about 16.2, about 16.3, about 16.4, about 16.5, about 16.6, about 16.7, about 16.8, about 16.9, about 17, about 17.1, about 17.2, about 17.3, about 17.4, about 17.5, about 17.6, about 17.7, about 17.8, about 17.9, about 18, about 18.1, about 18.2, about 18.3, about 18.4, about 18.5, about 18.6, about 18.7, about 18.8, about 18.9, about 18.9, about 19.0, about 19.1, about 19.2, about 19.3, about 19.4, about 19.5, about 19.6, about 19.7, about 19.8, about 19.9, about 20.0, about 20.1, about 20.2, about 20.3, about 20.4, about 20.5, about 20.6, about 20.7, about 20.8, about 20.9, about 21, about 21.1, about 21.2, about 21.3, about 21.4, about 21.5, about 21.6, about 21.7, about 21.8, about 21.9, about 22, about 22.1, about 22.2, about 22.3, about 22.4, about 22.5, about 22.6, about 22.7, about 22.8, about 22.9, about 23, about 23.1, about 23.2, about 23.3, about 23.4, about 23.5, about 23.6, about 23.7, about 23.8, about 23.9, about 24, about 24.1, about 24.2, about 24.3, about 24.4, about 24.5, about 24.6, about 24.7, about 24.8, about 24.9, about 25, about 25.1, about 25.2, about 25.3, about 25.4, about 25.5, about 25.6, about 25.7, about 25.8, about 25.9, about 26, about 26.1, about 26.2, about 26.3, about 26.4, about 26.5, about 26.7, about 26.8, about 26.9, about 27, about 27.1, about 27.2, about 27.3, about 27.4, about 27.5, about 27.6, about 27.8, about 27.9, about 28.0, about 28.1, about 28.2, about 28.3, about 28.4, about 28.5, about 28.6, about 28.7, about 28.8, about 28.9, about 29, about 29.1, about 29.2, about 29.3, about 29.4, about 29.5, about 29.6, about 29.7, about 29.8, about 29.9, about 30, about 30.1, about 30.2, about 30.3, about 30.4, about 30.5, about 30.6, about 30.7, about 30.8, about 30.9, about 31, about 31.1, about 31.2, about 31.3, about 31.4, about 31.5, about 31.6, about 31.7, about 31.8, about 31.9, about 32, about 32.1, about 32.2, about 32.3, about 32.4, about 32.5, about 32.6, about 32.7, about 32.8, about 32.9, about 33, about 33.1, about 33.2, about 33.3, about 33.4, about 33.5, about 33.6, about 33.7, about 33.8, about 33.9, about 34, about 35, about 36, about 37, about 38, about 39, about 40, about 41, about 42, about 43, about 44, about 45 about 46, about 47, about 48, about 48.5, about 49, about 49.2, about 49.5, about 49.2, about 49.5, about 50, about 50.5, about 51.0 about 51.5, about 52, about 52.5, about 53, about 54, about 55, about 56, about 57, about 58, about 59, about 60, about 65, about 70, about 75, about 80, about 85, about 90, about 95, about 100, about 105, about 106, about 107, about 108, about 108.6, about 109, about 109.5, about 110, about 111, about 112, about 115, about 120, about 125, about 130, about 130.5, about 131, about 131.5, about 132, about 132.5, about 133, about 135, about 140, about 145, and about 150, or any number ranging between any two of the values above, including at least about 3.0 to about 30.0, at least about 2.0 to about 4.0, at least about 4.0 to about 6.0, at least about 6.0 to about 8.0, at least about 8.0 to about 10.0, at least about 10.0 to about 12.0, at least about 12.0 to about 15.0, at least about 15.0 to about 20.0, at least about 20.0 to about 25.0, at least about 25.0 to about 30.0, at least about 30.0 to about 35.0, at least about 35.0 to about 40.0, at least about 40.0 to about 45.0, at least about 45.0 to about 50.0 at least about 50.0 to about 55.0, at least about 55.0 to about 60.0, at least about 60.0 to about 65.0, at least about 65.0 to about 70.0, at least about 70.0 to about 75.0, at least about 75.0 to about 80.0, at least 80.0 to about 85.0, at least about 85.0 to about 90.0, at least about 90.0 to about 95.0, at least about 95.0 to about 100.00, at least about 105.00 to about 110.00, at least about 110 to about 115.00, at least about 115.00 to about 120.00, at least about 120.00 to about 125.00, at least about 125.00 to about 130.00, at least about 130.00 to about 135.00, at least about 135.00 to about 140.00, at least about 140.00 to about 145.00, at least about 145.00 to about 150.00 times greater. The risk factor for recurrence can be based on a combination of odds ratios and pathological findings (see for example, Example 12A-12D).

In one embodiment, the PF is ECE. In one embodiment, the likelihood of recurrence is greater for a subject who is classified as PSA+ than for a subject who is classified as PSA− and ECE positive. In one embodiment, the likelihood of recurrence is greater for a subject who is classified as PSA+ than a subject who is classified as PSA− and ECE negative. (see for example, Example 12A-12D).

In one embodiment, the PF is surgical margins. In one embodiment, the likelihood of recurrence is greater for the subject who is classified as PSA+ than the subject who is classified as PSA− and surgical margins positive. In one embodiment, the likelihood of recurrence is greater for a subject who is classified as PSA+ than a subject who is classified as PSA− and surgical margins negative. (see for example, Example 12A-12D).

In one embodiment, the PF is a Gleason score. In one embodiment, the likelihood of recurrence is greater for a subject who is classified as PSA+ than a subject who is classified as PSA/− and a Gleason score of more than 7. In one embodiment, the likelihood of recurrence is greater for a subject who is classified as PSA+ than a subject who is classified as PSA− and a Gleason score of less than 7. (see for example, Example 12A-12D).

In one embodiment, the PF is a seminal vesicle. In one embodiment, the likelihood of recurrence is greater for a subject who is classified as PSA+ than a subject who is classified as PSA/− and seminal vesicle positive. In one embodiment, the likelihood of recurrence is greater for a subject who is classified as PSA+ than a subject who is classified as PSA− and a seminal vesicle negative. (see for example, Example 12A-12D).

This invention is also useful in monitoring patients treated for prostate disease, and the detection of prostate cancer, and cancer recurrence or stable disease following therapy, or following a decision not to administer post-prostatectomy therapy depending on pathological findings and the PSA values and PSA indices of this invention. The present invention has advantages over conventional serum PSA assays for identification of biochemical recurrence of prostate cancer following treatment by providing unique assays with limits of detection and functional sensitivities for PSA superior to conventional assays. This invention is therefore useful in the monitoring of patients treated for prostate disease and the detection of cancer recurrence as opposed to stable disease (absence or low likelihood of recurrence) following primary therapy such as RP.

The methods described herein are also useful, for example, in detecting early stage recurrence of prostate cancer or to make early determinations that a patient is stable or likely to have stable disease following radical prostatectomy for prostate cancer. The improved limit of detection and functional sensitivity of the present invention enables early detection of stable disease and/or recurrence and, in appropriate cases, enables earlier initiation of salvage and/or adjuvant treatment for recurring cancer.

Therapy for prostate cancer may include radical prostatectomy, radiation therapy, chemotherapy, or anti-androgen treatment. Measurement of [PSA]-time values associated with stable disease (sometimes referred to as detecting or determining stable disease) can avoid unnecessary adjuvant therapies in relatively young patients with poor margins and Gleason scores, who would otherwise be treated if stable disease were not detected. On the other hand, patients for whom recurrence is detected early using the methods of this invention, can undergo earlier treatment. Thus, the ability to detect low levels of PSA permits reduction of therapy of some patients currently being treated under current treatment regimes because they have a high probability of relapse, because conventional methods of measuring [PSA] cannot reliably determine that those patients have low PSA levels and low increases in the [PSA] level. See Nilsson et. al., Acta Oncologica Vol. 43, No. 4, pp 316-381, 2004.

In one embodiment the level or concentrations of total PSA (tPSA or PSA) can be monitored in a patient following therapy, by obtaining one or more samples from the patient after the therapy and determining the amount of PSA in each sample using a PSA assay having a detection limit at least as low as 1 pg/mL and a functional sensitivity lower than 10 pg/mL. In another embodiment, a PSA assay having a limit of quantitation and functional sensitivity of less than 1 pg/mL is used to determine recurrence of prostate cancer in a patient after therapy by determining whether a PSA time value exceeds its corresponding PSA index cutoff. In a more preferred embodiment the PSA assay has a limit of quantitation at least as low as 0.2 pg/mL and a functional sensitivity equal to or lower than 0.5 pg/mL.

The improved limit of detection and functional sensitivity of the PSA assays used in the methods of this invention permit detection of biochemical relapse or recurrence at an earlier stage. This detection of early stage biochemical recurrence should permit salvage and/or adjuvant treatment at an earlier stage, when there are fewer cancer cells and such cells may be more sensitive to treatment. Salvage and/or adjuvant treatments may include localized radiotherapy, and may be administered with or without concurrent androgen deprivation. For example, salvage and/or adjuvant radiotherapy has been shown to have a beneficial effect when used in treating men with PSA doubling times (the time in days or months or years when doubling of serum PSA concentration occurs) of less than 6 months, when the treatment was given <2 years after biochemical recurrence determined using standard conventional assays. Trock et al., ASCO 2008 Urogenitary Cancers Symposium, Abstract No. 85. In addition, detection of early stage biochemical recurrence may eliminate the need to conduct further costly management in patients who have stable disease, or avoid the need for unnecessary adjuvant and salvage therapies in those patients. In some embodiments of this invention, because the PSA-RPI is stronger risk identifier than clinical pathological factors, the determination of treatment will be based on whether the PSA time value exceeds the PSA-RPI, even if that determination of likelihood of recurrence or stable disease provides the opposite assessment of risk compared to one or more clinical pathological factors.

In another embodiment of this invention assays for PSA having a functional sensitivity of at least less than 1 pg/mL are used to detect biochemical recurrence at an early stage following therapy for prostate cancer. Indices based on PSA measurements are used in the detection of early stage biochemical recurrence. These indices include the maximum observed PSA level during monitoring, the nadir PSA level, a multiplier of the nadir PSA level, ratio of maximum observed PSA level to nadir PSA level, or the number of doublings. PSA rate indices such as velocity of PSA increase slope or use of Ln [PSA] vs. time, second consecutive increase, and doubling time can also be used. Any of these indices can be used singly or in combination in determining whether a patient has early stage biochemical recurrence (ES-BCR), or stable disease.

In one aspect the PSA assays embodied in this invention are used to determine whether a patient has an early risk for prostate cancer recurrence, i.e., to detect early stage biochemical recurrence (ES-BCR), or whether the patient is more likely to display stable disease characteristics, i.e., to detect stable disease. For example, if the maximum observed [PSA] is equal to or exceeds a [PSA] index, it is determined that the patient has ES-BCR, and if the maximum observed [PSA] is less than a [PSA] index, it is determined that the patient has stable disease.

As another example, PSA assays can be used to measure the PSA concentration level in serial samples obtained from a patient following radical prostatectomy for prostate cancer. The measurements can be used to determine a PSA rate value. By determining whether the PSA rate of increase value is equal to or exceeds the PSA rate of increase index, it is possible to detect whether the patient has ES-BCR or recurrence or stable disease. If the rate of increase in PSA is equal to or exceeds a rate index, it is determined that the patient has ES-BCR, and if the rate of increase in PSA falls below the threshold, it is determined that the patient has stable disease. When the PSA rate index is doubling time, the doubling time value is equal to or exceeds the doubling time index when the doubling time value is at least as low as the doubling time index, i.e. shorter doubling times are associated with poorer prognosis than higher doubling times.

In another aspect, further analysis based on one or more PSA indices permits classification of patients into additional sub-types, allowing clinicians to tailor treatments appropriate for that subtype and to use these therapies at an earlier time than current clinical practice. Early initiation of salvage and/or adjuvant treatment may improve patient outcomes.

The present invention will now be described more fully with reference to the accompanying figures and examples, which are intended to be read in conjunction with both this summary, the detailed description, and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete, and will fully convey the full scope of the invention to those skilled in the art.

### DESCRIPTION OF THE FIGURES

FIG. 1 displays results from one embodiment of this invention and specifically shows the plot of the Nucleic Acid Detection Immunoassay, ® [PSA] (PSA concentration) in pg/mL vs. days post radical prostatectomy for recurring patient number 11, with exponential fit. The ® assay [PSA] was the [PSA] determined in the ® assay study, described in the detailed description.

FIG. 2 shows the plot of the ® [PSA] in pg/mL vs. days post radical prostatectomy for recurring patient number 31.

FIG. 3 shows the plot of the ® [PSA] in pg/mL vs. days post radical prostatectomy for recurring patient number 38.

FIG. 4 shows the plot of the ® [PSA] in pg/mL vs. days post radical prostatectomy for stable patient number 86.

FIG. 5 shows the plot of the ® [PSA] in pg/mL vs. days post radical prostatectomy for stable patient number 120.

FIG. 6 shows the plot of the ® [PSA] in pg/mL vs. days post radical prostatectomy for stable patient number 126.

FIG. 7 shows the plots in pg/mL vs. days post radical prostatectomy for all 43 recurring patients are shown in the Figure.

FIG. 8 shows an overlay plot for 43 recurring patients, of [PSA] pg/ml vs time following prostatectomy with the PSA level range constrained to 1000 pg/ml.

FIG. 9 shows a plot of the first post-prostatectomy total [PSA] vs. the patient sub-population (recurrence of prostate cancer (1) or with stable disease (0)).

FIG. 10 shows a plot of the nadir total [PSA] vs. the patient sub-population (recurrence of prostate cancer (1) or with stable disease (0)).

FIG. 11 shows a plot of the maximum observed [PSA] level (pg/mL) vs. the patient sub-population (recurrence of prostate cancer (1) or with stable disease (0)).

FIG. 12 shows a plot of the maximum [PSA] level/nadir level [PSA] vs. the patient sub-population (recurrence of prostate cancer (1) or with stable disease (0)).

FIG. 13 shows a plot of the second consecutive increase in [PSA] level (pg/mL/month) vs. the patient sub-population (recurrence of prostate cancer (1) or with stable disease (0)).

FIG. 14 shows a plot of the doubling time data (days) vs. the patient sub-population (recurrence of prostate cancer (1) or with stable disease (0)).

FIGS. 15A-C show the overlay plots for recurring patients with doubling times of <150 days, 150-400 days, or >400 days, respectively.

FIG. 15A shows the overlay plots for recurring patients, of [PSA] pg/ml vs days post surgery with doubling times of <150 with range constrained to 1000 pg/mL

FIG. 15B shows the overlay plots for recurring patients, of [PSA] pg/ml vs days post surgery with doubling times of 150-400 with range constrained to 1000 pg/mL

FIG. 15C shows the overlay plots for recurring, of [PSA] pg/ml vs days post surgery patients with doubling times of >400 with range constrained to 1000 pg/Ml

FIGS. 16A-D shows the overlay plots for subclasses of recurring patients by doubling time, with ranges constrained to 1000 pg/mL, respectively. The recurring patients with doubling times of >400 days have been further subdivided whether the maximum observed PSA is above or below 200 pg/mL.

FIG. 16A shows the overlay plots for recurring patients with doubling time <150 days of [PSA] pg/ml vs days post surgery.

FIG. 16B shows the overlay plots for recurring patients with doubling time <150-400 days of [PSA] pg/ml vs days post surgery.

FIG. 16C shows the overlay plots for recurring patients with doubling time >400 days, maximum [PSA]>200 pg/mL vs days post surgery.

FIG. 16D shows the overlay plots for recurring patients [PSA] pg/ml vs days post surgery.

FIG. 17 shows the overlay plots of [PSA] pg/ml vs days post surgery that, with few exceptions, the stable disease patients generally have PSA maximums which do not exceed 15 pg/mL.

FIG. 18 shows a mosaic plot of the data showing the number of doublings during monitoring vs. the patient sub-population (recurrence of prostate cancer (1) or with stable disease (0)).

FIG. 19 shows a mosaic plot of the data showing the number of consecutive doublings vs. the patient subpopulation of recurrence of prostate cancer (1) or with stable disease (0).

FIGS. 20A and 20B show the multivariate ROC curve in comparison to the univariate ROC curve for the ® maximum observed [PSA] level. FIG. 20A shows the multivariate ROC curve. FIG. 20B shows the univariate ROC curve for the ® maximum observed [PSA] level (black line) vs. the multivariate ROC curve (dotted line).

FIGS. 21A and 21B show the multivariate ROC curve in comparison to the univariate ROC curve for the ® maximum total [PSA]/nadir [PSA] levels. FIG. 21A shows the multivariate ROC curve. FIG. 21B shows the univariate ROC curve for the ® maximum total [PSA]/nadir [PSA] levels (black line) vs. the multivariate ROC curve (dotted line).

FIGS. 22A and 22B show the multivariate ROC curve in comparison to the univariate ROC curve for the second rise in [PSA] (pg/mL/month). FIG. 22B shows the multivariate ROC curve. FIG. 22A shows the univariate ROC curve for the ® second rise in [PSA] (pg/mL/month) (black line) vs. the multivariate ROC curve (dotted line). Table 22 shows the results of the logistic regression and ROC computations.

FIGS. 23A-C show the univariate analysis for maximum total [PSA], second rise (pg/mL/month) indices, and maximum total [PSA]/nadir total [PSA].

FIG. 24 shows a linear curve fit for a stable patient for level of [PSA] (pg/mL) vs. time (months) over a time period of approximately eight years.

FIG. 25 shows a linear curve fit for a recurring patient for level of [PSA] (pg/mL) vs. time (months) over a time period of approximately five years.

FIG. 26: Kaplan-Meier plot of univariate survival probabilities for patients categorized as at reduced risk for clinical recurrence (dashed line) and not at reduced risk for recurrence (solid line) by PSA slope.

FIG. 27 is a schematic representation of an exemplary PSA-RPI assay

FIGS. 28A-C show plots of clinical PFS (progression free survival) probability vs. number of years of follow up post radical prostatectomy, for (A) low, (B) intermediate and (C) high multivariate risk groups with respect to ProsVue® results ([PSA]-time values and PSA RPI) from an exemplary PSA assay.

FIG. 29 is a synergy plot depicting univariate hazard ratio for clinical recurrence by Capra-S risk group and ProsVue® results.

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction. Additional embodiments may be disclosed in the Detailed Description below.

### DETAILED DESCRIPTION OF THE INVENTION

In one aspect this invention relates to certain uses of a recently developed and validated assay for measuring total serum PSA in human samples. In one aspect the assay is an immuno-polymerase chain reaction (IPCR), but other assays with low limits of quantitation (i.e., less than 2 pg/mL, 1.5 pg/mL, 1 pg/mL, 0.5 pg/mL or 0.2 pg/mL (or any limit of quantitation ranging between any of these values). In some aspects using a dual-antibody sandwich IPCR assay, a double-stranded (ds) DNA label can be used for sensitive analyte detection, and other features that reduce non-specific binding and improve analytical sensitivity and precision can also be used. For example, the limit of quantitation (LOQ) of a validated PSA dual-antibody sandwich IPCR assay of this invention is 0.00065 ng/mL (0.65 pg/mL), roughly an order of magnitude lower than other sensitive commercially available PSA assays. Further, in some aspects the assay employs an index based on the change in [PSA] over time post-RP as a unique prognostic factor. In some embodiments of this invention, because the PSA RPI is stronger risk identifier than clinical pathological factors, the determination of treatment will be based on whether the PSA time value exceeds the PSA-RPI, even if that determination of likelihood of recurrence or stable disease provides the opposite assessment of risk compared to one or more clinical pathological factors.

In one aspect, the assay used in these studies may be a multi-point assay, including a multi-point assay cleared by the Food and Drug Administration (FDA).

In addition, in some aspects the superior sensitivity (and low limits of detection and limits of quantitation) of the assays of this invention gave precise [PSA] determinations down to below 1 pg/mL and provided a unique opportunity to evaluate the kinetics of changes in [PSA] at these very low post-RP levels. In some aspects using the assays of this invention, the [PSA] change over time may exhibit first-order kinetics in non-recurring men, and the rate of change (in pg/mL/month) may be significantly different than for men who suffered from clinical recurrence. In some aspects values determined from the change in [PSA] over time may be used in the methods of this invention, such as slope [PSA], In (slope [PSA]), or a function, time function, statistical, mathematical or formulaic transformation, weighted PSA values, one or more value multiplier, or other expressions of a change in PSA over time.

In some aspects a [PSA] slope cutpoint, or [PSA] slope recurrence probability index (PSA-RPI) of ≦2.0 pg/mL/month, can be used to determine patients at a reduced risk of clinical recurrence. A slope function index, ln(slope) function index, velocity of change in [PSA] function index may be any amount between about 0.2 and 4.0 pg/mL/month, including, for example, about 0.2, about 0.3, about 0.4, about 0.5, about 0.6, about 0.7, about 0.8, about 0.9, about 1.0, about 1 about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.92, about 1.94, about 1.95, about 1.96, about 1.98, about 2.0, about 2.02, about 2.04, about 2.05, about 2.06, about 2.08, about 2.10, about 2.15, about 2.2, about 2.25, about 2.3, about 2.4, about 2.5, about 2.6 about 2.7, about 2.8, about 2.9, about 3.0, about 3.1, about 3.2, about 3.4, about 3.5, about 3.6, about 3.7, about 3.8, about 3.9, or about 4.0 pg/mL/month, or about any of those amounts, or about any range between any two of these values. As another example, the slope of Ln [PSA] vs. time index may be above about any level between 0.015 to 0.0425, preferably 0.015, 0.0175, 0.020, 0.0225, 0.025, 0.0275, 0.030, 0.0325, 0.035, 0.0375, 0.040, 0.0425, or 0.045, and more preferably 0.03. Studies using the assays of this invention, with an LOQ lower than 2 pg/mL, have determined that most men have serum PSA concentrations well below 10 pg/mL in the first several months following RP.

Prostate cancer is a complex disease, and it has proved difficult to accurately predict clinical outcomes in individual men following RP. Previous studies have linked undetectable PSA levels <0.01 ng/mL (<10 pg/mL) post-RP to relapse-free survival¹¹ and nadir values to a lower likelihood for BCR. However, the risk of BCR or clinical recurrence using assays with limits of quantitation higher than 2 pg/mL (such as 10 pg/mL or 5 pg/mL), is not informative as a predictor of clinical endpoints due to the apparently unpredictable kinetics of post-RP PSA values after a BCR event occurs—the result of trying to measure [PSA] levels below the limit of quantitation of those assays.

The assays according to this invention, can be used with an endpoint of clinically documented recurrence rather than BCR, to determine a PSA-RPI correlated with reduced risk of clinical recurrence. For example, in one embodiment, patients with a [PSA] slope value <a PSA-RPI of 2.0 pg/mL/month enjoyed a greater than 90% reduced risk for clinical recurrence. In a multivariate model that included pre-RP [PSA], pathologic stage and Gleason score, PSA-RPI slope was the most powerful independent prognostic factor for identifying men at reduced risk for clinical recurrence (HR 9.8, P<0.0001). While Gleason score (HR 5.4), was also a predictor, PSA-RPI slope was the strongest independent prognostic index of recurrence risk, including the subset of men having pathologic Gleason score ≧7.

According to one aspect of this invention, assays for total serum PSA having a limit of detection at least as low as 5 pg/mL, 2 pg/mL or 1 pg/mL (or any value between 5 and 1 pg/mL) and a limit of quantitation and/or functional sensitivity of less than 10 pg/mL are used to monitor patients following therapy for prostate cancer. The assays of this invention can be used to detect a likelihood of clinical recurrence (recurrence) and/or early stage biochemical recurrence following therapy and/or a likelihood of stable disease (or stable disease) post-surgery. Measuring the level of PSA refers to simultaneously measuring the level of total serum PSA, or tPSA.

However, there is a limitation even to the use of biochemical recurrence as an indicator of prostate cancer recurrence when conventional assays for PSA are used. The lowest values of serum PSA following radical prostatectomy are often below the limits of detection when conventional assays are used to measure PSA. See Junker et al., Anticancer Research 19:2625-2658 (1999). Thus, values of serum PSA following RP may be reported as zero nanograms/milliliter (ng/ml) with conventional assays when PSA is not actually absent in the circulation. See Stamey, Clin. Chem. 42(6): 849-852. Even if the PSA value is above the detection limit of a conventional assay, the concentration may nevertheless be below the assay's “functional sensitivity,” the ability to quantify concentrations of serum PSA at low levels with accuracy and precision. This means that the true nadir concentration of serum PSA either cannot be detected or cannot be reported with accuracy and precision by conventional assays. This is unfortunate since the nadir concentration itself may be a predictor of recurrence with lower nadir concentrations associated with lower likelihood of recurrence. Furthermore, if the serum PSA level should begin to rise, it may not be detectable by conventional assays until a time at which recurrence is at a stage when prognosis may again be poor.

Aggressive cancers may recur far more rapidly but conventional assays would not be able to detect these recurrences due to their limits of detection and insufficient functional sensitivity giving rise to scattered data when measuring [PSA] values below 10 pg/ml. Even non-aggressive cancers may begin to show a rise in serum PSA that is not detectable by conventional assays. Thus, conventional assays for serum PSA are not able to aid physicians in the early detection of prostate cancer recurrence.

Most current FDA cleared conventional PSA assays measure [PSA] down to approximately 100 pg/mL, and that limit of detection is reflected in the definition of biochemical recurrence recently recommended by the American Urological Association Prostate Cancer Guideline Panel ([PSA] of greater than 0.2 ng/mL (200 pg/mL), with a second confirmatory level of PSA greater than 0.2 ng/mL). See Cookson, et al., J. Urology 177:540-545 (2007). Due to the limitations in functional sensitivity, conventional PSA assays indicate the absence of PSA in samples having [PSA] below the functional sensitivity of the assays. E. G. Stamey (1996); Vassilikos et al., Clinical Biochemistry 33(2): 115-123 (2000).

For detection of early stage recurrence following therapy, it is of clinical importance to know whether PSA in post-therapy samples is within the functional sensitivity of an assay. Otherwise, clinicians and patients do not know whether a negative result reflects the “absence” of PSA or the limits of detection of the assay despite the presence of PSA-producing cells.

In the methods of this invention, assays having a low functional sensitivity limit as described herein have been used to measure PSA levels down to the 0.2-0.5 pg/mL range in serum samples from women. The 0.5 pg/mL functional sensitivity of the assay permitted determination that the levels of PSA in the sera of women are in the range of 0.5 to 3 pg/mL rather than zero, as was commonly assumed. In fact, the assays of this invention can be used to measure [PSA] in men following treatment such as radical prostatectomy below 1 pg/mL. Thus, the assays with a limit of detection down to 0.5 pg/mL are capable of measuring the lowest levels of PSA that one would expect to find in men post radical prostatectomy.

In some aspects, measuring PSA levels using PSA assays with a functional sensitivity of less than 0.5 pg/mL permits precise measurement of the low PSA levels in post-therapy prostate cancer patients. For example, measurement of [PSA] using the Nucleic Acid Detection Immunoassay (ProsVue®test) showed that following radical prostatectomy, many patients have stable low PSA levels, which indicates that those patients have very slow growing cancers or are cured. For patients displaying increased serum PSA levels with time, PSA levels could be accurately determined, enabling correlation of changes in [PSA] over time with clinical outcome. Patients' PSA serum levels were accurate enough to determine slopes for the increase in PSA, and to generate reproducible data for samples containing PSA levels previously below the functional sensitivity of current commercial assays. Measuring the level of PSA refers to measuring the level of total PSA, or tPSA.

Use of more sensitive PSA assays of this invention established that PSA levels increase exponentially following the post-RP nadir. The highly sensitive PSA measurements using the assays of this invention indicated that cancer cells were present and growing exponentially long before the [PSA] level reached 200 pg/mL. The results from a retrospective analysis of a dataset shows that prostate cancer cells are present and growing for a considerable length of time before the serum level reaches the current biochemical recurrence point of 200 pg/mL.

In one aspect of the invention a PSA assay having a limit of quantitation or functional sensitivity of at least as low as 0.5 pg/mL and/or a limit of detection 0.2 pg/mL is used to determine recurrence of prostate cancer at an early stage. It also decreases the time needed to detect early recurrence, up to, for example, 30 months earlier than with conventional assays. For example, the assays of this invention can be used to detect recurrence or stable disease as early as about 4, 5, 6, 7, 8, 9, 10, 11, 12, 13, 14, 15, 16, 17, 18, 19, 20, 21, 22, 23, 24, 25, 26, 27, 28, 29 or about 30 months following treatment. In some embodiments the assays of this invention can be used to detect recurrence or stable disease as early as about 4 to about 6 months, or by about 18 months or any range of time falling between any two of those times. Precise measurements of PSA in the 0.5 to 100 pg/mL range using these PSA assays also permits recognition of early stage biochemical recurrence and initiation of treatment much earlier than that based on current clinical practice.

Earlier detection of the need for salvage and/or adjuvant treatment for early stage recurrent prostate cancer decreases the time required to begin follow up treatment of patients, which generally currently takes place only after PSA levels exceed 200 pg/mL. As described herein, using PSA assays having a functional sensitivity of 0.5 pg/ml to monitor patients could lead to determination of [PSA] levels correlated with recurrence (i.e., detection of clinical recurrence), and evaluations for further therapy by 18 months, or at least as much as 30 months sooner than using current measures of biochemical recurrence. This will assist in providing earlier treatment when the cells are potentially more localized and/or susceptible to therapy.

In one aspect, the methods of this invention permit earlier and more accurate identification of men at risk for disease progression and patients with early treatment failure. The methods of this invention can also be used to earlier determine that the patient is not having a recurrence. As described herein, using PSA assays having a functional sensitivity of 0.5 pg/ml to monitor patients could lead to determination of [PSA] levels correlated with stable disease (i.e., detection of stable disease), and a determination that no further therapy is necessary at that time. The availability of more sensitive PSA assays therefore reduces system costs and patient anxiety by permitting earlier classification of patients as stable or having clinical recurrence or early biochemical recurrence.

In some aspects, the highly sensitive, early detection methods of this invention can be used in evaluating treatment options following radical prostatectomy. In some embodiments, this invention can be used to detect whether patients have recurrence or stable disease, whether, and how often patients should be monitored for recurrence, and whether and when salvage and/or adjuvant/treatments such as anti-androgen treatment, radiotherapy or chemotherapy should be administered.

Post prostatectomy treatments have been determined largely based on pathological findings such as Gleason score, age at diagnosis, surgical margins, T-stage, tissue invasion, extracapsular invasion, seminal vesicle invasion, bladder neck invasion, lymph node invasion, and tumor volume. Clinical parameters having predictive value for recurrence include high Gleason score, high PSA using current assays (above 200 ng/ml measured with current assays), pT3 disease, positive surgical margins, ECE, and seminal vesicle invasion.

A high percentage of patients with prostate cancer are not cured by RP, and 27-53% will display elevated [PSA] within 10 years. Nilsson et al., “A systematic overview of radiation therapy effects in prostate cancer,” Acta Oncologica, 43(4):316-381 (2004). However, between 30% and 70% of the patients currently treated with adjuvant therapy will not suffer from recurrence. Thus, administering adjuvant and/or salvage therapy to post-prostatectomy patients on the basis of pathological findings such as age, Gleason score and surgical margins alone may expose a significant percentage of patients who have stable disease to unnecessary, costly treatments and potential complications.

As an example, adjuvant treatments may be administered to patients displaying poor clinical signs. These patients include relatively young patients with poor margins and Gleason scores. For instance, patients in their fifties having poor margins and Gleason scores of greater than or equal to 7, will usually undergo therapy such as external radiotherapy (RT). Post-prostatectomy treatment with external beam radiotherapy in patients with stage pT3 disease prolongs biochemical disease-free survival, and the likelihood of achieving stable disease in patients who are not cured by RP is higher when treatment is given earlier, rather than delayed salvage and/or adjuvant treatment. See Nilsson et al., at 316.

However, use of the highly sensitive assays and [PSA] values and indices of this invention can be used alone or in combination with pathological findings to provide early detection of stable disease, and can avoid unnecessary adjuvant therapies currently being administered. For example, early detection of stable disease in relatively young patients who would otherwise be treated, can avoid the need for unnecessary treatments, and attendant risk of side effects. Side effects of post-prostatectomy therapy can include incontinence, urinary frequency, nocturia, cystitis, diarrhea, rectal bleeding, decreased libido and/or impotence. Accordingly, in some aspects, early detection of stable disease using the detection methods of this invention can avoid unnecessary adjuvant therapies in patients who routinely receive adjuvant therapies based on pathological findings. On the other hand, delaying salvage/adjuvant treatment until the [PSA] obtained using conventional methods reaches 200 pg·mL diminishes the likelihood of achieving stable disease. See Nilsson at 345.

Thus, in some aspects, the PSA values and PSA indices of this invention are used in combination with pathological findings to determine whether adjuvant and/or salvage treatment should be administered. Additionally, because the [PSA] time values of this invention are more tightly correlated with stable disease then absence of clinicopathological observations, whether the [PSA] time value exceeds the PSA-RPI may determine the course of treatment. For example if adjuvant and/or salvage therapy would normally be administered to a patient based on one or more clinicopathological observations, but one or more PSA values does not exceed the PSA index, and stable disease is thereby detected, then unnecessary treatment could be avoided. PSA values and indices that can be used in these methods are described throughout. As a more specific example, when the slope of [PSA]-time value is lower than the PSA-RPI, then even if a relatively young patient has poor margins and a Gleason score of >7, adjuvant treatment can be avoided, and the patient monitored until one or more [PSA] time values exceeds the PSA-RPI.

In other aspects, when the methods of this invention are used in combination with pathological findings to detect early stage recurrence, patients diagnosed as having a PSA-time value exceeding a PSA-RPI (i.e., recurrence) can undergo earlier treatment, leading to increased prognosis. Radiation and chemotherapy can be performed according to methods and protocols known to those of skill in the art. Anti-androgen treatment can be performed using drug and biologic drug compositions, combinations, dosage forms and dosages known to those of ordinary skill in the art for adjuvant or salvage therapy in the treatment of post-prostatectomy patients.

An example of a PSA assay having a functional sensitivity of about 0.5 pg/mL and a detection limit of 0.2 pg/mL according to this invention is a sandwich format immunoassay using polymerase chain reaction (PCR) for signal generation. An example of such an assay useful in detecting PSA in serum or plasma samples in the methods of this invention is described below. Any PSA assay having a functional sensitivity of least as low as specified such as the ones described herein may be used. Methods for detecting proteins and for signal generation in protein assays are known to those in the art. For example, the methods of this invention may use other assay formats, including sandwich immunoassay formats, and any method of signal generation capable of providing the required functional sensitivity for use in the methods of this invention. For example, the methods of signal generation may include use of deoxyribonucleic acid (DNA), bioluminescence, radioactivity, chemifluorescence, nanoparticles, aptamers, or oligo-nanoparticles, either singly or in combination.

In addition, as discussed in more detail below, clinicopathalogical observations, or other PSA values such as doubling time and/or maximum observed PSA concentration can be used to further classify early stage recurring patients into multiple groups. These classifications could potentially be used to recommend different therapies for patients in the different subgroups. Thus, use of the methods of this invention will provide clinicians and patients with an accurate indication of treatment failure or early stage biochemical recurrence, and will permit more timely and appropriate selection of therapies to control the disease. In addition, earlier treatment therapy as a result of early detection may improve patient outcomes and avoid the need for more costly management of patients having stable disease.

In one embodiment, this invention includes a method of detecting whether a patient has early stage biochemical recurrence (ES-BCR), clinical recurrence or stable disease, comprising a) obtaining a sample from a patient after therapy for prostate cancer; b) measuring the PSA level in the sample using a PSA assay having a functional sensitivity at least as low as 20 pg/mL, c) using the PSA level from one or more samples to determine a PSA value, wherein ES-BCR is detected if the PSA value exceeds a PSA index in one or more samples.

The assay for PSA can be used to determine the PSA level in samples taken from a patient following a treatment for prostate cancer. PSA level may include the amount or concentration of PSA in the sample or a function thereof. The sample may be a plasma or serum sample. Measurements of PSA levels and/or a PSA-time value determined from the PSA level measurements may be used to monitor and assess whether therapy for prostate cancer has effectively treated the disorder. Preferably, the PSA assay has a limit of quantitation or functional sensitivity at least as low as 0.5 pg/mL and a limit of detection as low as 0.2 pg/mL.

The observed PSA level may be a concentration or amount or function thereof. A “PSA time value” is a parameter that is a function of the observed PSA level. PSA time value may include, for example, the slope of Ln [PSA] vs. time, the velocity of increase in PSA level, the doubling time for PSA level. In some aspects a PSA value such as the ratio of the observed PSA level or maximum observed PSA level to the nadir PSA level, or the second consecutive increase in PSA level may also be used. As used herein, PSA time value can include, for example, any function, statistical, mathematical or formulaic transformation, weighted PSA values, one or more value multiplier, or other expressions of a change in PSA over time.

A “PSA recurrence probability index” (PSA-RPI) is a predetermined cutoff, threshold or number, which discriminates with or without statistical significance between subpopulations of patients having stable disease and patients having, or who will have recurrence. As used herein, PSA recurrent probability index (RPI) can include, for example, any function, statistical, mathematical, mathematical or formulaic transformation, one or more weighted PSA values, one or more multiplier, expressions of the numerical and/or statistical predictive indication. In other aspects, the degree of dependence between PSA-RPI and recurrence or stable disease may also be determined.

An index based on PSA-RPI and one or more pathological factors (PSA-RPI-PF) such as Gleason score, age at diagnosis, surgical margins, T-stage, tissue invasion, extracapsular invasion, seminal vesicle invasion, bladder neck invasion, lymph node invasion, biopsy, or tumor volume may also be used to further discriminate, with or without statistical significance, between subpopulations of patients having stable disease or likely to have stable disease, and patients having recurrence, or likely to have recurrence. In other aspects, the degree of dependence between PSA-RPI-PF and recurrence or stable disease may also be determined.

As used herein, “correlate” or “correlation” between PSA recurrence probability index and risk of recurrent presentation can include a statistical predictive indication describing the high degree of dependence between PSA-RPI and recurrent risk factors. In a certain embodiment, the predictive indication can be a linear or non-linear correlation coefficient. In certain embodiment, a correlation coefficient of greater than about or 0.78 or 0.90 is observed.

As used herein, a PSA+ or PF+ classification denote the presence or satisfaction of the threshold clinicopathological condition or numerical and/or statistical parameter.

Recurrence is detected when one or more selected PSA time values obtained using a PSA assay with a limit of detection at least as low as 1 pg/mL exceed the corresponding PSA index (which is correlated with recurrence). The values and corresponding indices can be used singly or in combination in determining whether a patient has recurrence or stable disease.

Disease recurrence may be determined biochemically, or based on clinical observations and/or pathological findings such as imaging or biopsy, although those methods suffer from poor sensitivity for recurrence. One or more of the PSA values and PSA indices obtained using the methods of this invention can also be used in combination with pathological findings to facilitate or determine treatment options for patients. For example, detection of recurrence using the methods of this invention may result in further therapy, including radiation therapy, chemotherapy or anti-androgen therapy. In some instances, further therapy may be warranted if there is an early, rapid, increase in a [PSA] value, and/or if an early measured PSA rate value exceeds a PSA rate index. As another example, an early, less rapid [PSA] rate increase may or may not result in further therapy, depending on other patient parameters including clinical and/or pathological findings and/or observations.

Clinical or pathological findings and/or observations may include Gleason score, age at diagnosis, surgical margins, T-stage, tissue invasion, extracapsular invasion, seminal vesicle invasion, bladder neck invasion, lymph node invasion, biopsy, or tumor volume can also be determined. In some embodiments, the parameters supporting further therapy include age less than an age cutoff, a Gleason score exceeding a Gleason score cutoff, high PSA using the methods of this invention, positive surgical margins, ECE and seminal vesicle invasion. The age cutoff may be, for example, 50, 51, 52, 53, 54, 55, 56, 57, 58, 59 or 60, 65, 70, 75 or 80 or any value ranging between any two ages. The Gleason score cutoff may be, for example, 4, 5, 6, 7, 8, 9, 10 or any value ranging between any two of those scores. As used herein, PF can include, for example, any function, statistical, mathematical or formulaic transformation, weighted values, value multiplier, expressions of the numerical and/or statistical predictive indication based on clinical or pathological findings.

As another example, a slow increase in a [PSA] rate value may not result in further therapy, if pathological findings indicative of lack of recurrence such as low Gleason score, or advanced age (such as over 70 or 80), are also present. In addition, if the methods of this invention detect stable disease, no further therapy will be administered. In any instance where further therapy is not administered, it may be desirable to further monitor one or more PSA values using the methods of this invention, either alone or in combination with pathological findings, to determine if further therapy should be administered at a later time.

A PSA recurrence probability index (RPI) may be a predetermined cutoff or threshold for a PSA time function such as [PSA] slope, the slope of Ln [PSA] vs. time, the velocity of increase or rate of rise in PSA, or the doubling time for PSA. Doubling time is (Ln(2)/K), where K is the slope of the exponential fit of a plot of PSA level versus time. In the case of doubling time, the PSA time value “exceeds” the PSA RPI when the doubling time value is less than or equal to the PSA index. In certain embodiments, the slope is a smooth function.

PSA indices may include the maximum observed PSA level, a multiplier of the nadir PSA level, the maximum observed PSA level, or the nadir PSA level, which may be determined using standard statistical methods such as those described herein. As an example, the PSA level index may be a [PSA] index of at least about A PSA level of at least any level between 10 to 60, or 10 to 99 pg/mL, preferably 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 99 pg/mL and any range between any two levels. More preferably, the PSA level index may be a [PSA] index of at least about 15 pg/mL, 20 pg/mL or 25 pg/mL. A PSA level index range may also be specified. A [PSA] index range may be, for example, 10 to 20 pg/mL, 15-25 pg/mL, 15-22 pg/mL or 20-25 pg/mL. The PSA level index may be used alone or in combination with other PSA indices or clinical indices to determine patients having stable disease or ES-BCR.

By “PSA nadir” is meant the lowest measured amount of PSA in a sample from the patient following therapy such as radical prostatectomy. The PSA nadir results from clearance of PSA produced by proliferating prostate tissue removed or killed during treatment. PSA has a half life of 2.2 days to 3.5 days, and may take from 3 to 4 weeks or up to 6-8 weeks to clear from the bloodstream. Ellis et al., Adult Urology, 50 (4), 573-579, (1997). Following treatment such as radical prostatectomy, the serum PSA level decreases to a nadir following treatment which removes or kills the proliferative prostatic cells. In patients with stable disease, the PSA levels may remain flat after reaching a low point. The sample may be one of a serial set of blood serum samples for which PSA level is measured. A serial set of blood serum samples is two or more samples taken at different time points from the same patient following therapy such as radical prostatectomy or salvage and/or adjuvant treatment.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, horses, mice; rats; rabbits; guinea pigs; dogs; cats; sheep; goats; cows; primates, such as monkeys, chimpanzees, and apes, and humans.

As used herein, prostate disease can include, for example, prostate cancer, and/or an adenocarcinoma or an adenocarinoma that has migrated to the bone. Exemplary prostate cancer develops in the prostate organ in men, which surrounds the first part of the urethra. Other exemplary prostate cancers may include adenocarcinomas that develop in the glandular cells responsible for generating seminal fluid.

As used herein, the term “treatment” or “therapy” refers to both primary, salvage, and/or adjuvant therapeutic modalities and prophylactic or preventative measures, or administering an agent suspected of having therapeutic potential. Treatment may refer to one or more therapeutic modalities in the clinical management of prostate disease. A treatment for a prostate disease can include, for example, a treatment for prostate cancer. A treatment for prostate cancer can include, for example, surgery and/or radical prostatectomy (RP). Treatment for prostate cancer may also include immunotherapy, radiation therapy, salvage radiation therapy, as well as hormonal or chemotherapies, or anti-androgen modalities or combined modalities.

As used herein, exemplary surgical procedures to treat prostate cancer can include, radical prostatectomy, for example, radical retropubic prostatectomy, a radical perineal prostatectomy, cryosurgery, and a laparoscopic radical prostatectomy.

As used herein, exemplary radiation can include, for example, brachytherapy (seed implantation or interstitial radiation therapy), external beam radiation, including three dimensional conformal radiation therapy, intensity modulated radiation therapy, and conformal proton beam radiation therapy.

As used herein, anti-androgen modalities can include, for example, surgery castration, chemical castration, and/or hormone therapy. Exemplary hormone therapy can include androgen deprivation therapy (ADT) and/or androgen suppression therapy. The goal is to reduce levels of male hormones, called androgens, in the body, or to prevent them from reaching prostate cancer cells. Exemplary androgens are testosterone and dihydrotestosterone (DHT). Exemplary surgery can also include orchiectomy in which the testicles, where 90% of androgens are produced, are removed.

Exemplary hormone therapy can also include, for example, administration of luteinizing hormone-releasing hormone (LHRH) analogs to lower androgen levels. Exemplary LHRH analogs can include leuprolide, goserelin, triptorelin, and histrelin. Treatment with these drugs is sometimes called chemical castration because they lower androgen similar to surgical castration, e.g. orchiectomy.

An LHRH antagonist may also be administered, such as abarelix.

Anti-androgens block the body's ability to use any androgens. Even after orchiectomy or during treatment with LHRH analogs, the adrenal glands still make small amounts of androgens. Exemplary anti-androgens can include, for example, flutamide (Eulexin®), bicalutamide (Casodex®), and nilutamide (Nilandron®). Anti-androgen treatment may be combined with orchiectomy or LHRH analogs as first-line hormone therapy. This is called combined androgen blockade (CAB).

Additional exemplary androgen modulating agents can include, for example, estrogens (female hormones); Ketoconazole (Nizoral®); Ketoconazole can block the production of cortisol, an important steroid hormone in the body; Abiraterone (Zytiga®): Abiraterone blocks an enzyme called CYP17, which helps stop these cells from making certain hormones, including androgens; DES, MDV3100 and orteronel,

As used herein, the term “agonist” is defined as a compound that increases the activity of a receptor when it contacts the receptor.

As used herein, the term “antagonist” is defined as a compound that competes with an agonist or inverse agonist for binding to a receptor, thereby blocking the action of an agonist or inverse agonist on the receptor. However, an antagonist (also known as a “neutral” antagonist) has no effect on constitutive receptor activity.

As used herein, the term “inverse agonist” is defined as a compound that decreases the basal activity of a receptor (i.e., signaling mediated by the receptor). Such compounds are also known as negative antagonists. An inverse agonist is a ligand for a receptor that causes the receptor to adopt an inactive state relative to a basal state occurring in the absence of any ligand. Thus, while an antagonist can inhibit the activity of an agonist, an inverse agonist is a ligand that can alter the conformation of the receptor in the absence of an agonist. The concept of an inverse agonist has been explored by Bond et al. in Nature 374:272 (1995). More specifically, Bond et al. have proposed that unliganded beta 2-adrenoceptor exists in an equilibrium between an inactive conformation and a spontaneously active conformation. Agonists are proposed to stabilize the receptor in an active conformation. Conversely, inverse agonists are believed to stabilize an inactive receptor conformation.

As used herein, “partial agonists” can bind and activate a given receptor, but have only partial efficacy at the receptor relative to a full agonist. Partial agonists can also be considered as ligands which display both agonistic and antagonistic effects—when both a full agonist and partial agonist are present, the partial agonist may act as a competitive antagonist, competing with the full agonist for receptor occupancy and producing a net decrease in the receptor activation observed with the full agonist alone.

As used herein, “full agonists” bind and activate a receptor, they have affinity for the receptor and display full efficacy in activating the receptor upon binding.

As used herein, pathological findings (PF) or clinicopathological findings can include, for example, any suitable factors known in the art or may later develop for useful to determine post-RP risk stratification such as, for example, Gleason score, percentage of grade 4-5 disease, surgical margin involvement, extracapsular extension (ECE), seminal vesicle invasion and lymph node involvement, as well as multivariate models (nomograms) that incorporate these factors.

As used herein, presence or absence (+/−) seminal vesicle invasion (SVI) is a pathological finding that is a significant risk factor for clinical recurrence of prostate cancer post-prostatectomy. Determination of PSA-RPI slope adds significantly more information to this risk assessment. In certain embodiments, the odds of clinical recurrence in men with SVI and a PSA-RPI slope >2.0 pg/mL/month is 4.88 times higher compared to men with SVI and a PSA-RPI slope ≦2.0 pg/mL/month. Likewise, in certain embodiments, the odds of clinical recurrence in men without SVI and a PSA-RPI slope >2.0 pg/mL/month are 15.47 times higher compared to men without SVI and a PSA-RPI slope ≦2.0 pg/mL/month.

As used herein, the pathological finding based on Gleason score (pGS) (e.g. ≧7) is a significant risk factor for clinical recurrence of prostate cancer post-prostatectomy. In certain embodiments, the odds of clinical recurrence in men with a pGS ≧7 are 8.31 times higher compared to men with a pGS <7. Determination of PSA-RPI slope adds significantly more information to this risk assessment. In certain embodiments, the odds of clinical recurrence in men with a pGS ≧7 and a PSA-RPI slope >2.0 pg/mL/month is 27.0 times higher compared to men with a pGS ≧7 and a PSA-RPI slope ≦2.0 pg/mL/month. (See for example, Example 12A-D)

As used herein, the pathological finding based on presence or absence (+/−) of extracapsular extension (ECE) is a significant risk factor for clinical recurrence of prostate cancer post-prostatectomy. As used herein, extracapsular extension (ECE) can include, for example, capsular extension. Determination of PSA-RPI slope adds significantly more information to this risk assessment. In certain embodiments, the odds of clinical recurrence in men with ECE and a PSA-RPI slope >2.0 pg/mL/month is 6.06 times higher compared to men with ECE and a PSA-RPI slope ≦2.0 pg/mL/month. Likewise, In certain embodiments, the odds of clinical recurrence in men without ECE and a PSA-RPI slope >2.0 pg/mL/month are 29.7 times higher compared to men without ECE and a PSA-RPI slope ≦2.0 pg/mL/month.

As used herein, the pathological findings based on a positive surgical margin (+SM) is a significant risk factor for clinical recurrence of prostate cancer post-prostatectomy. Determination of PSA-RPI slope adds significantly more information to this risk assessment. In certain embodiments, the odds of clinical recurrence in men with +SM and a PSA-RPI slope >2.0 pg/mL/month is 5.88 times higher compared to men with a +SM and a PSA-RPI slope ≦2.0 pg/mL/month. Likewise, In certain embodiments, the odds of clinical recurrence in men with a negative surgical margin (−SM) and a PSA-RPI slope >2.0 pg/mL/month are 21.57 times higher compared to men with a −SM and a PSA-RPI slope ≦2.0 pg/mL/month.

An assay for total PSA preferably has a detection limit at least as low as 10 pg/mL and a functional sensitivity at least as low as 20 pg/mL. A PSA assay may preferably have a functional sensitivity of at least as low as about 15, 14, 13, 12, 11, 10, 9, 8, 7, 6, 5, 4, 3, 2, 1, 0.9, 0.8, 0.7, 0.6, or 0.5 pg/mL. A PSA assay may preferably have a detection limit as low as 0.2 pg/mL and/or a functional sensitivity of about 0.5 pg/mL. The detection limit is alternatively referred to herein as functional detection limit or limit of detection. The limit of detection (LOD) is the lowest amount of analyte in a sample that can be detected with type I and II error rates set to 5%

In some embodiments the limit of detection can be at least as low as 15, 14, 13, 12, 11, 10, 9, 8, 7, 6, 5, 4, 3, 2, 1, 0.9, 0.8, 0.7, 0.6, 0.5 or 0.2 pg/mL. The PSA assay may also have a detection limit as low as 0.5 pg/mL, with a functional sensitivity as low as 1, 2, 3, 4, or 5 pg/mL. In some embodiments, the PSA assay has a functional detection limit of 0.2 pg/mL and a functional sensitivity of 0.5 pg/mL, and further comprises contacting the sample with a conjugate comprising a non-nucleic acid PSA binding entity and a nucleic acid marker that can be used to generate a PCR signal.

The most common definition of biochemical recurrence recently is a [PSA] of greater than 0.2 ng/mL (200 pg/mL), although levels ranging from 100 to 2000 pg/mL have been used. Doherty et al., J. Cancer 83(11): 1432-1436 (2000). With a PSA assay having a functional sensitivity of at least as low as 1.0 pg/mL, it is possible to determine whether or not early stage biochemical recurrence has occurred. The detection of early stage biochemical recurrence takes place earlier than detection of conventionally defined biochemical recurrence using conventional PSA assays.

In one aspect of the invention, ES-BCR based on PSA level may be detected by comparison of the maximum observed PSA level to a PSA level index. A PSA level of at least any level between 10 to 60 pg/mL, at least any level between 10 to 60, or 10 to 99 pg/mL, preferably 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 99 pg/mL and any range between any two levels, and more preferably 10, 15, 20, 25, or 30 pg/mL can be used as a PSA index to determine ES-BCR. For example, if the maximum observed PSA level index is 15 pg/mL, then comparing a maximum observed PSA level greater than 15 pg/mL to the PSA index detects ES-BCR.

In another aspect, the maximum observed PSA to nadir PSA ratio may be used as the PSA index for determining whether ES-BCR has occurred. For example, the maximum observed PSA/nadir PSA may be any number between 3 to 11, preferably 3, 4, 5, 6, 7, 8, 9, 10, or 11, and more preferably 6. The multiplier of the nadir PSA level may be 2×, 4×, or 8×, preferably 4×.

In another embodiment of this invention, assays for PSA can be used to determine PSA in serial samples taken from a patient following therapy. PSA measurements from the serial samples taken over time can be used to calculate PSA rate values including velocity of the change in PSA, the slope of Ln [PSA] vs. time, or the doubling time for the increase in PSA. Comparison or one or more of these PSA rate values to its corresponding rate index permits determination of whether ES-BCR has occurred.

In this embodiment, the invention may comprise, for example, methods for determining whether a patient has early stage biochemical relapse (ES-BCR), comprising:

a) obtaining serial samples from the patient; b) determining the PSA level in each sample using a PSA assay having a functional sensitivity at least as low as 1 pg/mL; c) determining that the PSA rate value exceeds a PSA rate index, thereby detecting ES-BCR; or determining that the PSA rate value does not exceed the rate index, thereby detecting that the disease is stable.

The first sample for use in determining a PSA value may be taken at any time after therapy, and at or following the clearance of pre-therapy PSA levels and the PSA nadir. Generally, the first sample will be taken any time between 2 weeks to 8 weeks following treatment. Samples may be taken at any set of intervals used in the clinical monitoring of prostate disease. Preferably the first sample will be taken 30 or 45 days after treatment, with subsequent samples preferably taken at 3 month intervals. This time course may be modified if the PSA value of a sample indicates that ES-BCR has occurred or indicates treatment failure.

The rate of rise in PSA level can be measured from the point of the PSA nadir. Patients whose velocity of increase in PSA rises above an index level can be characterized as undergoing early stage biochemical recurrence. The rate indices can be obtained, evaluated, or determined by using statistical analyses including univariate logistic regression and receiver-operating characteristic (ROC) analysis, bivariate analysis or multivariate analysis or other appropriate statistical methods to obtain index values that provide good discrimination between patient subpopulations having stable disease and ES-BCR.

As discussed further below, the rate of rise in PSA levels over time is a good index of whether the patient has ES-BCR, recurrence, or stable disease. In addition, a rate index such as the second consecutive rise may be used as an index of whether the patient has ES-BCR or stable disease. The velocity of change in [PSA] index or the second consecutive rise index may be any amount between about 0.2 and 4.0 pg/mL/month, including, for example, about 0.2, about 0.3, about 0.4, about 0.5, about 0.6, about 0.7, about 0.8, about 0.9, about 1.0, about 1 about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.92, about 1.94, about 1.95, about 1.96, about 1.98, about 2.0, about 2.02, about 2.04, about 2.05, about 2.06, about 2.08, about 2.10, about 2.15, about 2.2, about 2.25, about 2.3, about 2.4, about 2.5, about 2.6 about 2.7, about 2.8, about 2.9, about 3.0, about 3.1, about 3.2, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, or about 6.8 pg/mL/month, or about any amount in between any two of those amounts. The increase in the PSA-time value can be used to confirm or modify the pathological findings during recurrence risk assessment. As another example, the slope of Ln [PSA] vs. time index may be above about any level between 0.015 to 0.0425, preferably 0.015, 0.0175, 0.020, 0.0225, 0.025, 0.0275, 0.030, 0.0325, 0.035, 0.0375, 0.040, 0.0425, or 0.045, and more preferably 0.03.

In addition, in one embodiment a doubling time index of whether a patient may be any number of days between 400-800 days, more preferably 400, 425, 450, 475, 500, 525, 550, 575, 600, 625, 650, 675, 700, 725, 750, 775 or 800 days, and most preferably 550 days. When the PSA rate value does not exceed the PSA rate index, the determination is made that the disease is stable; if the PSA rate value is equal to or exceeds the PSA rate index, recurrence is detected. In the case of a doubling time index, recurrence will be determined if the PSA doubling time in days is equal to or less than the doubling time index. For the doubling time value and index, the doubling time value will be determined to exceed the doubling time index if the doubling time value is less than the doubling time index.

In other embodiments, the maximum observed PSA index and PSA time index are used in combination to determine whether a patient has stable disease or ES-BCR. For example, the method may further comprise c) determining that that the PSA level is above a PSA index of 5, 10, 15, or 20 pg/mL (or any range or value in between 10 and 20 pg/mL) and that the slope of [PSA] vs. time value is above a slope of [PSA] vs. time index of about 2.0 pg/mL/month in order to detect recurrence. On the other hand, if the PSA level is less than a PSA index or the rate of rise in [PSA] vs. time is below about 2.0 pg/mL/month, stable disease is detected. In this example, stable disease is determined if both the PSA time value is equal to or exceeds the PSA time index, and the observed PSA value is equal to or exceeds the maximum observed PSA index.

In another aspect, the invention is a method of detecting whether a patient has fast (e.g., slope >5.0), moderately fast (e.g., slope >2.0), moderate (e.g., slope >1.0), moderately slow (e.g., slope >0.5), or slow (e.g., slope >0.2) recurrence or biochemical recurrence (ES-BCR), comprising a) obtaining a serial set of blood serum samples from a patient after therapy for prostate cancer; b) measuring the PSA level in each sample using a PSA assay having a limit of detection of about 0.5 pg/mL; c) determining a PSA rate value; d) determining that the PSA time value is equal to or less than a PSA-time index, thereby detecting recurrence or ES-BCR; and e) classifying recurrence or ES-BCR as rapid, medium, or slow based on the PSA rate index. (see for example, Example 12A-D)

Patients whose PSA doubling time value is equal to or exceeds the rate threshold may be classified as having fast, moderately fast, moderate, moderately slow, or slow recurrence or ES-BCR based on doubling time. As an example, in some embodiments, a doubling time equal to or less than about ten months indicates fast or rapid recurrence; a doubling time of more than about ten months up to equal to or about 24 months indicates medium ES-BCR, and a doubling time of more than about 24 months indicates slow recurrence.

In another aspect, the invention is a method of detecting whether a patient has moderately fast, moderate, moderately slow, or slow recurrence or ES-BCR, comprising a) obtaining a serial set of blood serum samples from a patient after therapy for prostate cancer; b) measuring the PSA level in each sample using a PSA assay having a functional sensitivity of about 0.5 pg/mL; c) determining the doubling time value and the maximum observed PSA value; d) determining that the doubling time is equal to or less than a doubling time index, thereby detecting recurrence or ES-BCR; and e) classifying ES-BCR as rapid, medium, or slow based on the doubling time and maximum observed PSA.

In other aspects, patients whose PSA doubling time value is equal to or exceeds the doubling time index may be classified into four subclasses of ES-BCR based on doubling time and/or maximum observed PSA. Type 1 recurring patients have a doubling time of less than 150 days. Type 2 recurring patients have a doubling times between 150-400 days, Type 3 recurring patients and type 4 recurring patients have PSA doubling times greater than 400 days. For Type 3 patients, the maximum observed PSA exceeds 200 pg/mL, while Type 4 patients have maximum observed PSA values which do not exceed 200 pg/mL for an extended time of longer than 400 days.

In another aspect, the invention is a method of detecting if a patient has early stage biochemical recurrence (ES-BCR) after salvage and/or adjuvant therapy for prostate cancer, comprising a) obtaining a samples from the patient after salvage therapy; b) measuring the PSA level in the sample using a PSA assay having a functional sensitivity of about 0.5 pg/mL; c) using the PSA level from one or more samples to determine a PSA value; wherein ES-BCR is detected if the PSA value exceeds a PSA index and stable disease is detected if the PSA value does not exceed the PSA index.

In another aspect the invention is a kit comprising a nucleic acid-anti-PSA conjugate suitable for performing a sandwich immunoassay for PSA using PCR signal detection, wherein the assay has a detection limit at least as low as 0.2 pg/mL and a limit of quantitation or functional sensitivity at least as low as 0.5 pg/mL. The kit may further comprise software for determining one or more PSA values.

In another aspect, the invention is a label comprising a description of a method of detecting whether a patient has early stage biochemical relapse (ES-BCR), comprising
a) obtaining a sample from a patient after therapy for prostate cancer; b) measuring the PSA level in the sample using a PSA assay having a functional sensitivity less than 1 pg/mL; c) using the PSA level from one or more samples to determine a PSA value; wherein ES-BCR is detected if the PSA value is equal to or exceeds a PSA index and stable disease is detected if the PSA value does not exceed the PSA index.

### EXAMPLES

The invention is now described with reference to the following Examples. These Examples are provided for the purpose of illustration only, and the invention is not limited to these Examples, but rather encompasses all variations that are evident as a result of the teaching provided herein.

Prior to the experiments described herein, there was no published protocol that allows for determining the risk of prostate cancer recurrence in a subject following treatment for prostate disease using a combination of PSA recurrence probability index and pathological findings. The highly sensitive assays of this invention permit accurate and precise measurements of [PSA] values at low levels following treatment for PSA cancer, including within relatively short time frames from treatment. This permits a high correlation (statistically significant) between a patient's PSA time value exceeding a PSA time index and the patient having a risk for recurrence (detection of recurrence). This further permits a very high correlation (statistically significant) between a patient's PSA time value not exceeding a PSA time index and a likelihood of stable disease (detection of stable disease).

Other aspects and embodiments of the instant disclosure are based on the surprising and unexpected discovery of the synergistic predictive power of combining PSA time values obtained using the highly sensitive assays of this invention with a PSA recurrence probability index and pathological findings, in order to determine the risk of prostate cancer recurrence or stable disease in a subject following treatment for prostate disease. This novel method enables, for the first time, a highly predictive and highly sensitive validated assay for assessing recurrence risk or likelihood of stable disease in subjects having a post-treatment level of PSA of <100 pg/mL, which were previously below the limit of conventional assay detection

In the experiments described herein, several factors were discovered that allowed the unexpected enhanced/potentiated predictive efficacy for prostate cancer recurrence. For example, it was discovered that the combination of the PSA-RPI index and the PF findings was the most powerful index of reduced risk of clinical recurrence and added predictive value to established risk factors. This method could possibly reduce healthcare costs by reducing the intensity of follow-up in men identified at a reduced risk for recurrence.

Without being bound to a particular theory of operation, the skilled artisans will appreciate that pathological findings which can be suitably combined with the PSA-RPI index of this method to yield these advantageous predictive parameters are also contemplated herein.

By way of example, clinical studies using a higher sensitivity assay for total PSA (tPSA) showed that biochemical recurrence can be detected earlier by monitoring changes in serum PSA using the higher sensitivity assays. In contrast, the functional sensitivity of previously reported conventional assays is limited and cannot reliably report PSA levels less than around 0.01 ng/mL (10 pg/mL). Thus, use of high sensitivity [PSA] assays provides more reliable, early detection of recurrence.

### Example 1

### Nucleic Acid Detection Immunoassay (PSA-RPI Assay) for the Detection of Very Low Levels of Prostate Specific Antigen (PSA)

Total PSA (tPSA) in serum samples was measured using an exemplary assay of this invention, which was a nucleic acid detection immunoassay (PSA-RPI assay) having a functional sensitivity of 0.5 pg/mL. See Clin Chem 53(6) Suppl., 2007, #C-15. The exemplary PSA RPI assay is performed in sandwich immunoassay format.

Two antibodies directed to different epitopes on PSA were employed in an assay designed to detect pg/mL levels of PSA in patient samples from men who have undergone radical prostatectomy.

### Example 1A

### Production of Signal Nucleic Acid-Anti-PSA Conjugate

The first antibody is conjugated (chemically linked) to an oligonucleotide of 60 bases as described by Jablonski and Adams in IVD Technology, November 2006. This reporter antibody is then diluted to approximately 10-30 picomolar (pM) concentration in a buffered diluent containing bovine serum albumin (BSA) and a surfactant to decrease non-specific binding at a pH range of 7.0-7.5.

### Example 1B

### Production of Capture Nucleic Acid-Anti-PSA Conjugate

The second antibody is immobilized on a para-magnetic particle of approximately 1 micron in diameter. The capture antibody has biotin chemically attached to it, using EZ-Link Sulfo-NHS-LC-Biotin (Sulfosuccinimidyl-6-(biotinamido) hexanoate, Catalog Number 21335 as supplied by Pierce using methods described in their catalog, and is subsequently bound to the para-magnetic particle through a streptavidin linker that has been attached to the magnetic particle by the manufacturer, Seradyn (Catalog Number 3015-2104).

### Example 1C

### Conditions for ® Assays

75 microliters (μl) of reporter antibody is allowed to react with 20 μl of patient serum sample for two hours at room temperature. In a heterogeneous format, the capture antibody, immobilized on the para-magnetic particles, is then added to the reporter antibody and sample solution. This mixture is allowed to react for 30 minutes with mild agitation to keep the para-magnetic particles in suspension.

At the end of this incubation the particles are separated magnetically from the remaining solution which is carefully removed leaving the magnetic particles on the side of the well. The magnetic particles are then washed 3-5 times removing non-bound reporter antibody. This solution is buffered at neutral pH containing a surfactant such as Tween 20. The result is a washed particle containing only PSA, if present, sandwiched by a capture antibody and a reporter antibody labeled with DNA.

PCR reagent containing complementary primers to the DNA and Taq polymerase is then added to the washed para-magnetic particles and real time PCR is performed. This PCR amplification step uses standard commercially available reagents. In the presence of an immune-complex, which contains DNA bound to the reporter antibody, amplification of the DNA template Occurs.

The unknown sample is then read from a standard curve generated from calibrators of known tPSA concentration, 5, 25 and 100 pg/mL. Additionally each 96 well plate contains controls at 0.0, 10.0, and 80.0 pg/mL of PSA further ensuring the PCR amplification step is under proper control for each plate run.

As described in Jablonski and Adams in IVD Technology, November 2006, the assay can also be run in a homogenous format. For example, a first anti-PSA monoclonal antibody was labeled with an oligonucleotide sequence (a), and the second antibody was conjugated to oligonucleotide sequence (b) or (c). Oligonucleotide sequence (a) was complementary to the sequences (b) and (c), for the last 9 and 15 bases, respectively, at the 3′ ends. The conjugate pair was diluted to 10-100 pmol in 10 mmol Tris (pH 8.0) containing 0.1% bovine serum albumin (BSA) and combined in the presence of PSA for 2 hours. The solution was then diluted with Tris/BSA to reduce the bulk conjugate concentration to below 1 pmol and was held at 52° C. for 1 minute to fully melt unbound conjugate. PCR reagent mixture, containing Taq polymerase and downstream primers, was added, and the reaction was sealed. The temperature was lowered to 23° C. to fully hybridize the DNA strands associated with the immune complex and to initiate the first chain extension. Free MAb-DNA cannot hybridize to the same degree in the time frame of the first extension in dilute solution, and cannot participate in subsequent exponential amplification. The overlapping DNA labels that were associated with the PSA immune complex were extended for 5 minutes, and completed by increasing the temperature to 85° C. over 3 minutes. Real-time PCR amplification of the formed template was begun immediately, destroying the immune complex, which was no longer needed. The sensitivity of the assay was determined to be about 100 fg/mL.

To demonstrate the performance of the PSA RPI assay, IMD obtained patient samples from the Lab of Eleftherios Diamandis M. D., Ph.D. (University Health Network and Toronto Medical Laboratories, Toronto, ON, Canada). These samples included 42 patients which were previously characterized by Dr. Diamandis as stable and 43 patients with rising PSA values which he classified as having a biochemical recurrence. The samples were obtained post prostatectomy and were included if their PSA values post surgery dropped below 100 pg/mL. A biochemical recurrence was defined using several criteria and were based on time point values obtained during the course of the study. See Yu, He; Diamandis, Eleftherios, P. Wong, Pui-Yuen; Nam, Robert; Trachtenberg, John “Detection of Prostate Cancer Relapse with Prostate Specific Antigen Monitoring at Levels of 0.001 to 0.1 ug/L” J. Urology 157:913-18 (1997).

The ® PSA assay was sensitive enough to precisely distinguish tPSA values in all female samples and the lowest observed values in the samples from the male population in the retrospective clinical study from background values.

### Example 2

### Retrospective Study to Evaluate Indiceslndices of Disease Outcome

® assays were used to measure tPSA levels in serial serum samples from prostate cancer patients following radical prostatectomy. The results were compared to earlier measurements of the PSA levels in the serum samples using a research assay based on an immunofluorometric (IFM) assay. Vassilikos et al., Clin. Biochem. 33: 115-123 (2000). The 0 assay results were then analyzed to determine concordance with the patient's clinical outcome.

Samples

Serum samples (N=435) stored following a previously published study (J Urol 157:913-8, 1997) were used in this study. The samples were collected in 1993 and 1994, PSA levels were measured using the Abbott Laboratories IMx assay, and the samples were stored frozen at −40° C. The samples were also used in the study by Vassilikos et al. where the IFM assay was used to determine tPSA levels. The IFM assay is further described in Clin Chem 39:2108-14, 1993. The serum samples used in the Vassilikos et al. study were obtained from 85 patients who had baseline tPSA <100 pg/mL post-RP (measured using the IMx assay), and who each had more than 3 serial samples taken post-RP (mean 5.0, median 5, range 3-6). Median (range) age was 63 years (49-73), pre-RP tPSA was 7.1 ng/mL (0.1-49.0), Gleason score was 7 (5-9) and % tumor involvement was 25% (1-90%). Clinical stages were T1a-c (16), T2a-b (35) and unknown (33). 4 patients received pre-RP therapy (hormones=1; radiotherapy=2). In the Journal of Urology study, the serum samples for which tPSA values were originally determined with the Abbott IMx assay were re-analyzed by the IFM method and showed no significant differences compared to original values. The Journal of Urology article defined BCR as ≧2 successive tPSA increases reaching ≧100 pg/mL, with relapse backdated to the first tPSA increase.

Serum samples from post-radical prostatectomy (RP) patients were included in the ® assay PSA study if their PSA levels after a RP were below the detectable limit using currently FDA cleared conventional PSA assays. Many of the conventional assays report that a patient has a zero or <0.1 ng/mL (<100 pg/mL) value post surgery. The ® PSA assay can detect approximately a 200 fold lower level of PSA than the FDA cleared PSA assays. Therefore, use of a higher sensitivity PSA assay permitted for the first time the measurement of the true level of PSA in post prostatectomy patients. The more sensitive and precise measurement of PSA levels allowed placement of patients into two groups—stable disease and early stage biochemical relapse.

Descriptive Statistics for Patients in the Study

Seven patients were prospectively excluded from this analysis, because no ® assay data were available or no surgery data was available. The final number of patients included in this study was eighty-five (85). Measurements of [PSA] (pg/mL) obtained by time of sampling for each patient included in the study are shown in Table 1, below.

| | Recurrence | | |
|---|---|---|---|
| Patient | (1 = Yes, | Days Post- | |
| ID | 0 = No) | Surgery | pg/ml PSA |
| 11 | 1 | 970 | 4.23 |
| | 1 | 1285 | 42.37 |
| | 1 | 1517 | 1255.62 |
| | 1 | 1708 | 2680.00 |
| 28 | 1 | 229 | 30.79 |
| | 1 | 550 | 109.86 |
| | 1 | 915 | 350.69 |
| | 1 | 1364 | 319.66 |
| | 1 | 1721 | 502.86 |
| 31 | 1 | 452 | 88.02 |
| | 1 | 660 | 159.86 |
| | 1 | 807 | 156.12 |
| | 1 | 2067 | 1859.00 |
| | 1 | 2431 | 2008.00 |
| 38 | 1 | 112 | 5.43 |
| | 1 | 224 | 17.10 |
| | 1 | 329 | 58.69 |
| | 1 | 763 | 189.08 |
| | 1 | 1444 | 883.84 |
| | 1 | 1666 | 1322.65 |
| 41 | 1 | 375 | 6.35 |
| | 1 | 508 | 10.41 |
| | 1 | 882 | 15.08 |
| | 1 | 1069 | 20.68 |
| | 1 | 1264 | 23.65 |
| | 1 | 1701 | 73.83 |
| 60 | 1 | 891 | 9.38 |
| | 1 | 1031 | 5.76 |
| | 1 | 1459 | 11.21 |
| | 1 | 1859 | 18.17 |
| | 1 | 2202 | 21.62 |
| | 1 | | |
| 64 | 1 | 460 | 44.30 |
| | 1 | 845 | 102.10 |
| | 1 | 1036 | 132.20 |
| | 1 | 2224 | 278.80 |
| 65 | 1 | 644 | 18.38 |
| | 1 | 806 | 25.68 |
| | 1 | 1565 | 114.02 |
| | 1 | 2011 | 216.38 |
| | 1 | 2150 | 278.46 |
| | 1 | 2312 | 388.57 |
| 79 | 1 | 938 | 147.10 |
| | 1 | 1281 | 155.80 |
| | 1 | 1366 | 193.70 |
| | 1 | 1557 | 197.80 |
| | 1 | 1731 | 271.80 |
| | 1 | 1974 | 2357.00 |
| 87 | 1 | 583 | 10.50 |
| | 1 | 751 | 9.69 |
| | 1 | 1081 | 17.63 |
| | 1 | 1458 | 35.58 |
| | 1 | 2192 | 105.97 |
| 89 | 1 | 424 | 97.80 |
| | 1 | 772 | 143.25 |
| | 1 | 857 | 221.32 |
| | 1 | 998 | 330.06 |
| | 1 | | 752.68 |
| 92 | 1 | 155 | 41.87 |
| | 1 | 301 | 54.52 |
| | 1 | 429 | 119.80 |
| | 1 | 513 | 153.72 |
| | 1 | 1785 | 1406.41 |
| 97 | 1 | 557 | 75.88 |
| | 1 | 698 | 455.62 |
| | 1 | 1264 | 542.54 |
| | 1 | 1672 | 726.70 |
| 103 | 1 | 716 | 29.12 |
| | 1 | 1243 | 6.45 |
| | 1 | 1621 | 65.48 |
| | 1 | 1781 | 164.06 |
| 105 | 1 | 655 | 10.52 |
| | 1 | 879 | 23.36 |
| | 1 | 1863 | 295.16 |
| | 1 | 2226 | 399.07 |
| 108 | 1 | 385 | 56.15 |
| | 1 | 887 | 306.78 |
| | 1 | 1224 | 378.35 |
| | 1 | 1586 | 661.77 |
| 113 | 1 | 540 | 4.57 |
| | 1 | 928 | 8.66 |
| | 1 | 1320 | 18.69 |
| | 1 | 1730 | 49.04 |
| | 1 | 2258 | 78.79 |
| 124 | 1 | 275 | 167.90 |
| | 1 | 631 | 331.40 |
| | 1 | 716 | 636.40 |
| | 1 | 1974 | 1782.00 |
| 136 | 1 | 81 | 9.70 |
| | 1 | 340 | 72.74 |
| | 1 | 515 | 184.14 |
| | 1 | 1757 | 647.86 |
| 151 | 1 | 188 | 39.13 |
| | 1 | 432 | 62.72 |
| | 1 | 830 | 169.00 |
| | 1 | 1061 | 382.34 |
| 160 | 1 | 248 | 10.84 |
| | 1 | 346 | 6.63 |
| | 1 | 528 | 24.15 |
| | 1 | 794 | 67.76 |
| | 1 | 976 | 122.64 |
| | 1 | 1354 | 228.00 |
| 177 | 1 | 1196 | 0.39 |
| | 1 | 1375 | 20.17 |
| | 1 | 1674 | 1.60 |
| | 1 | 2193 | 52.22 |
| | 1 | 2204 | 1.37 |
| 179 | 1 | 863 | 8.87 |
| | 1 | 1236 | 16.11 |
| | 1 | 1635 | 35.70 |
| | 1 | 2006 | 40.80 |
| | 1 | 2335 | 57.90 |
| 183 | 1 | 15 | 13.51 |
| | 1 | 218 | 77.75 |
| | 1 | 1041 | 255.50 |
| | 1 | 1375 | 520.09 |
| 184 | 1 | 281 | 6.59 |
| | 1 | 960 | 43.05 |
| | 1 | 1131 | 61.97 |
| | 1 | 1302 | 93.32 |
| | 1 | 1711 | 1722.20 |
| 197 | 1 | 490 | 42.80 |
| | 1 | 905 | 129.10 |
| | 1 | 1329 | 446.10 |
| | 1 | 1476 | 357.10 |
| | 1 | 1813 | 1585.30 |
| 214 | 1 | 184 | 20.66 |
| | 1 | 310 | 53.05 |
| | 1 | 1257 | 108.69 |
| | 1 | 1677 | 178.18 |
| | 1 | 2039 | 248.77 |
| 230 | 1 | 48 | 8.13 |
| | 1 | 138 | 10.36 |
| | 1 | 230 | 9.61 |
| | 1 | 671 | 38.85 |
| | 1 | 1588 | 201.80 |
| 242 | 1 | 128 | 8.69 |
| | 1 | 285 | 12.78 |
| | 1 | 582 | 82.14 |
| | 1 | 1163 | 178.67 |
| | 1 | 1541 | 277.47 |
| 261 | 1 | 47 | 13.21 |
| | 1 | 608 | 2227.87 |
| | 1 | 720 | 3267.70 |
| | 1 | 1026 | 60.10 |
| | 1 | 1132 | 241.10 |
| | 1 | 1385 | 2920.06 |
| 262 | 1 | 722 | 22.47 |
| | 1 | 1114 | 77.27 |
| | 1 | 1488 | 612.22 |
| | 1 | 1688 | 217.07 |
| | 1 | 1849 | 171.23 |
| 282 | 1 | 147 | 31.72 |
| | 1 | 793 | 171.80 |
| | 1 | 1165 | 299.61 |
| | 1 | 1362 | 678.51 |
| 300 | 1 | 48 | 4.70 |
| | 1 | 192 | 34.70 |
| | 1 | 350 | 108.80 |
| | 1 | 445 | 222.98 |
| | 1 | 592 | 267.07 |
| | 1 | 864 | 578.09 |
| 301 | 1 | 112 | 1.60 |
| | 1 | 265 | 3.12 |
| | 1 | 623 | 14.22 |
| | 1 | 833 | 27.30 |
| 302 | 1 | 54 | 6.48 |
| | 1 | 122 | 41.39 |
| | 1 | 410 | 528.84 |
| | 1 | 577 | 805.97 |
| | 1 | 748 | 941.12 |
| | 1 | 921 | 1302.18 |
| 303 | 1 | 86 | 3.45 |
| | 1 | 385 | 5.30 |
| | 1 | 545 | 14.10 |
| | 1 | 748 | 13.99 |
| | 1 | 1031 | 43.80 |
| | 1 | 1437 | 78.12 |
| 308 | 1 | 87 | 4.60 |
| | 1 | 177 | 19.93 |
| | 1 | 545 | 93.62 |
| | 1 | 744 | 196.28 |
| | 1 | 1028 | 295.98 |
| | 1 | 1210 | 484.54 |
| 309 | 1 | 60 | 1.27 |
| | 1 | 346 | 1.84 |
| | 1 | 756 | 2.02 |
| | 1 | 1188 | 81.39 |
| 312 | 1 | 188 | 35.89 |
| | 1 | 261 | 26.73 |
| | 1 | 391 | 258.85 |
| | 1 | 572 | 9338.12 |
| | 1 | 678 | 13316.08 |
| 322 | 1 | 155 | 4.41 |
| | 1 | 597 | 43.57 |
| | 1 | 839 | 87.82 |
| | 1 | 1128 | 180.12 |
| | 1 | 1241 | 255.53 |
| | 1 | 1601 | 315.70 |
| 325 | 1 | 101 | 1.36 |
| | 1 | 224 | 1.65 |
| | 1 | 686 | 5.59 |
| | 1 | 866 | 9.33 |
| | 1 | 1112 | 15.13 |
| | 1 | 1474 | 30.93 |
| 337 | 1 | 110 | 3.52 |
| | 1 | 482 | 1.31 |
| | 1 | 580 | 14.20 |
| | 1 | 671 | 69.91 |
| | 1 | 881 | 230.07 |
| | 1 | 1255 | 348.08 |
| 340 | 1 | 52 | 58.17 |
| | 1 | 71 | 79.29 |
| | 1 | 113 | 149.15 |
| | 1 | 393 | 476.20 |
| | 1 | 505 | 568.08 |
| | 1 | 1149 | 11857.37 |
| 29 | 0 | 108 | 5.15 |
| | 0 | 276 | 3.59 |
| | 0 | 473 | 7.85 |
| | 0 | 646 | 3.68 |
| | 0 | 1718 | 1.65 |
| 37 | 0 | 947 | 2.43 |
| | 0 | 1107 | 1.37 |
| | 0 | 1275 | 3.28 |
| | 0 | 1808 | 2.42 |
| | 0 | 2494 | 3.93 |
| 81 | 0 | 368 | 3.68 |
| | 0 | 712 | 2.49 |
| | 0 | 1084 | 4.37 |
| | 0 | 1516 | 2.03 |
| | 0 | 1716 | 3.38 |
| 82 | 0 | 755 | 9.47 |
| | 0 | 958 | 4.29 |
| | 0 | 1128 | 3.99 |
| | 0 | 1394 | 2.98 |
| | 0 | 2185 | 1.91 |
| 86 | 0 | 492 | 2.73 |
| | 0 | 667 | 2.22 |
| | 0 | 858 | 3.41 |
| | 0 | 1031 | 2.60 |
| | 0 | 1545 | 2.91 |
| 100 | 0 | 1288 | 1.42 |
| | 0 | 1652 | 3.35 |
| | 0 | 2030 | 1.99 |
| | 0 | 2770 | 1.20 |
| | 0 | 3133 | 1.38 |
| 120 | 0 | 638 | 2.48 |
| | 0 | 806 | 2.18 |
| | 0 | 977 | 0.82 |
| | 0 | 1150 | 3.52 |
| | 0 | 1536 | 1.65 |
| 126 | 0 | 585 | 6.34 |
| | 0 | 892 | 1.34 |
| | 0 | 1477 | 0.79 |
| | 0 | 1896 | 3.39 |
| | 0 | 2166 | 3.89 |
| | 0 | 2273 | 1.03 |
| 128 | 0 | 212 | 8.11 |
| | 0 | 331 | 3.56 |
| | 0 | 513 | 1.60 |
| | 0 | 605 | 2.67 |
| | 0 | 1788 | 2.31 |
| 137 | 0 | 202 | 4.19 |
| | 0 | 356 | 1.44 |
| | 0 | 541 | 1.09 |
| | 0 | 723 | 1.88 |
| | 0 | 1078 | 1.46 |
| | 0 | 1416 | 0.87 |
| 144 | 0 | 203 | 3.13 |
| | 0 | 359 | 1.94 |
| | 0 | 532 | 5.09 |
| | 0 | 994 | 3.89 |
| | 0 | 1392 | 5.47 |
| | 0 | 1815 | 1.73 |
| 154 | 0 | 842 | 1.79 |
| | 0 | 1444 | 0.85 |
| | 0 | 1528 | 2.23 |
| | 0 | 1808 | 2.03 |
| | 0 | 2235 | 1.51 |
| | 0 | 2403 | |
| 164 | 0 | 315 | 5.77 |
| | 0 | 539 | 4.97 |
| | 0 | 1316 | 6.00 |
| | 0 | 1703 | 4.10 |
| | 0 | 1983 | 6.01 |
| 167 | 0 | 877 | 17.35 |
| | 0 | 1231 | 22.01 |
| | 0 | 1926 | 24.20 |
| | 0 | 2226 | 127.05 |
| 178 | 0 | 181 | 0.19 |
| | 0 | 251 | 0.15 |
| | 0 | 469 | 0.21 |
| | 0 | 1007 | 3.62 |
| | 0 | 1387 | 4.68 |
| | 0 | 1578 | 0.12 |
| 191 | 0 | 61 | 2.19 |
| | 0 | 256 | 1.36 |
| | 0 | 727 | 1.19 |
| | 0 | 987 | 6.27 |
| | 0 | 1385 | 1.17 |
| | 0 | 1687 | 2.72 |
| 193 | 0 | 61 | 5.56 |
| | 0 | 152 | 3.09 |
| | 0 | 277 | 6.43 |
| | 0 | 999 | 10.37 |
| | 0 | 1196 | 7.27 |
| 196 | 0 | 33 | 4.31 |
| | 0 | 537 | 1.97 |
| | 0 | 922 | 2.25 |
| | 0 | 1289 | 3.33 |
| | 0 | 1634 | 4.29 |
| 219 | 0 | 257 | 1.34 |
| | 0 | 700 | 7.71 |
| | 0 | 852 | 1.95 |
| | 0 | 1444 | 1.38 |
| 227 | 0 | 49 | 4.40 |
| | 0 | 235 | 4.13 |
| | 0 | 353 | 8.60 |
| | 0 | 446 | 25.08 |
| | 0 | 616 | 6.80 |
| | 0 | 790 | 8.00 |
| 231 | 0 | 1243 | 5.28 |
| | 0 | 1564 | 10.53 |
| | 0 | 1923 | 14.46 |
| | 0 | 2292 | 14.79 |
| | 0 | 2657 | 13.99 |
| 235 | 0 | 57 | 1.93 |
| | 0 | 87 | 4.76 |
| | 0 | 196 | 4.33 |
| | 0 | 415 | 6.34 |
| | 0 | 570 | 4.49 |
| | 0 | 967 | 4.05 |
| 244 | 0 | 299 | 2.10 |
| | 0 | 516 | 2.55 |
| | 0 | 760 | 3.27 |
| | 0 | 969 | 5.26 |
| | 0 | 1146 | 2.03 |
| | 0 | | 3.17 |
| 246 | 0 | 104 | 4.48 |
| | 0 | 229 | 11.95 |
| | 0 | 391 | 8.40 |
| | 0 | 761 | 4.13 |
| | 0 | 1104 | 3.64 |
| | 0 | 1498 | 5.24 |
| 254 | 0 | 118 | 1.23 |
| | 0 | 166 | 1.59 |
| | 0 | 811 | 3.11 |
| | 0 | 1154 | 2.58 |
| 255 | 0 | 1321 | 3.60 |
| | 0 | 1477 | 3.17 |
| | 0 | 1607 | 3.93 |
| | 0 | 1883 | 4.32 |
| | 0 | 2175 | 2.50 |
| | 0 | 2525 | 9.68 |
| 259 | 0 | 75 | 1.60 |
| | 0 | 173 | 2.44 |
| | 0 | 393 | 2.74 |
| | 0 | 581 | 2.14 |
| | 0 | 1042 | 1.90 |
| | 0 | 1526 | 2.89 |
| 265 | 0 | 175 | 5.01 |
| | 0 | 742 | 1.18 |
| | 0 | 1115 | 0.87 |
| | 0 | 1615 | 0.80 |
| 266 | 0 | 55 | 3.78 |
| | 0 | 191 | 3.90 |
| | 0 | 321 | 6.76 |
| | 0 | 697 | 5.05 |
| | 0 | 1035 | 4.87 |
| | 0 | 1480 | 14.39 |
| 280 | 0 | 428 | 2.08 |
| | 0 | 616 | 2.37 |
| | 0 | 990 | 0.71 |
| | 0 | 1401 | 1.02 |
| | 0 | 1813 | 2.20 |
| 285 | 0 | 220 | 1.01 |
| | 0 | 591 | 3.06 |
| | 0 | 955 | 0.98 |
| | 0 | 1147 | 1.27 |
| | 0 | 1343 | 0.81 |
| | 0 | 1493 | 3.47 |
| 290 | 0 | 91 | 1.38 |
| | 0 | 210 | 2.54 |
| | 0 | 478 | 3.73 |
| | 0 | 842 | 3.81 |
| | 0 | 1037 | 2.75 |
| | 0 | 1420 | 5.25 |
| 296 | 0 | 131 | 4.28 |
| | 0 | 552 | 3.06 |
| | 0 | 798 | 1.83 |
| | 0 | 976 | 3.34 |
| | 0 | 1178 | 1.01 |
| | 0 | 1464 | 1.23 |
| 305 | 0 | 55 | 2.39 |
| | 0 | 328 | 1.74 |
| | 0 | 738 | 2.66 |
| | 0 | 951 | 2.18 |
| | 0 | 1140 | 1.69 |
| | 0 | 1418 | 2.19 |
| 313 | 0 | 37 | 4.46 |
| | 0 | 95 | 3.86 |
| | 0 | 199 | 6.51 |
| | 0 | 472 | 7.71 |
| | 0 | 815 | 6.17 |
| | 0 | 1144 | 6.00 |
| 317 | 0 | 719 | 3.09 |
| | 0 | 930 | 0.76 |
| | 0 | 1094 | 1.01 |
| | 0 | 1315 | 0.89 |
| | 0 | 1749 | 1.18 |
| | 0 | 2543 | 3.58 |
| 321 | 0 | 91 | 0.92 |
| | 0 | 242 | 0.74 |
| | 0 | 641 | 0.70 |
| | 0 | 1005 | 1.13 |
| | 0 | 1440 | 1.77 |
| 326 | 0 | 24 | 2.40 |
| | 0 | 252 | 1.47 |
| | 0 | 426 | 1.49 |
| | 0 | 860 | 0.75 |
| | 0 | 1180 | 3.86 |
| | 0 | 1298 | 4.62 |
| 330 | 0 | 69 | 5.06 |
| | 0 | 524 | 5.64 |
| | 0 | 624 | 5.16 |
| | 0 | 820 | 6.80 |
| | 0 | 1016 | 7.71 |
| | 0 | 1234 | 6.56 |
| 336 | 0 | 75 | 1.66 |
| | 0 | 256 | 1.64 |
| | 0 | 599 | 2.45 |
| | 0 | 788 | 1.13 |
| | 0 | 958 | 1.07 |
| | 0 | 1313 | 1.61 |
| 341 | 0 | 58 | 19.33 |
| | 0 | 165 | 7.84 |
| | 0 | 382 | 4.49 |
| | 0 | 697 | 4.77 |
| | 0 | 1137 | 3.97 |
| | 0 | 1270 | 2.86 |
| 347 | 0 | 785 | 1.96 |
| | 0 | 1179 | 4.29 |
| | 0 | 1366 | 2.81 |
| | 0 | 1555 | 2.90 |
| | 0 | 1793 | 3.64 |
| | 0 | | |

Forty-three (43) were classified as recurring and forty-two (42) were classified as having stable disease based on the Diamandis research assay. Yu, et al., J. Urology 157:913-18 (1997). Clinicopathological variable descriptive statistics for the patient populations were obtained. Significance of differences in the clinical variables distribution between patients in recurring and stable disease classifications are summarized in Table 2 below (p<0.05 indicated a significant difference in the distribution of the variable between the recurring population and the stable disease population.

TABLE 2
Clinicopathological variables - significance of differences in distribution
between recurring and stable disease patients

| Variable | N | p |
|---|---|---|
| Age at diagnosis | 68 | 0.6117* |
| Stage | 51 | 0.3324** |
| Gleason Score | 66 | 0.0276** |
| Pre-op chemotherapy | 55 | 0.1611** |
| Treatment Type | 51 | 0.4216** |
| Margin involved | 61 | 0.0006** |
| Peri prostatic tissue invasion | 51 | 0.0006** |
| Capsular invasion | 62 | 0.0181** |
| Seminal vesicle invasion | 62 | 0.6216** |
| Bladder neck invasion | 51 | 0.7037** |
| Lymph node involved | 60 | n/a |
| Tumor volume | 56 | 0.0008* |

*Wilcoxon rank sum
**Chi-square

In the current study, eighty-four (98.8%) of the patients were evaluable for biochemical evaluation using ® assays to measure tPSA, and 60-70% of them were evaluable clinicopathologically. Measuring tPSA using ® showed that the median (range) nadir or first tPSA value post-RP was 4.1 pg/mL (0.2-167.9 pg/mL).

In addition, as shown in the table above of the significance of differences in the distribution of clinical variables between recurring and stable disease classifications: Gleason, Surgical margin, Peri-prostatic invasion, Capsular invasion, and Tumor volume all show significant differences between sub-populations and may be predictors of outcomes.

### Example 3

### Evaluation of [PSA] Based Measurements as Index(s) of Disease Outcome

Analysis of the data collected for the sample set permitted evaluation of hypotheses that various PSA measurement indices were predictive of disease outcome, and would be useful in monitoring patients following therapy for prostate cancer. These indices included the following values based on ® assay measurements of tPSA in serial samples from patients: tPSA doubling time (calculated only from patients for whom ® assay PSA values were capable of exponential fitting); first post-prostatectomy level (the nadir value is not always the same as the first post-prostatectomy value); maximum tPSA level observed post-nadir (can be at any point in monitoring); ratio of maximum tPSA level to nadir (requires at least one value higher than the apparent nadir level at some time point after the nadir to indicate a possible recurrence); second consecutive increase pg/mL/month; rate of increase; number of doublings during the monitoring period; number of consecutive doublings during monitoring.

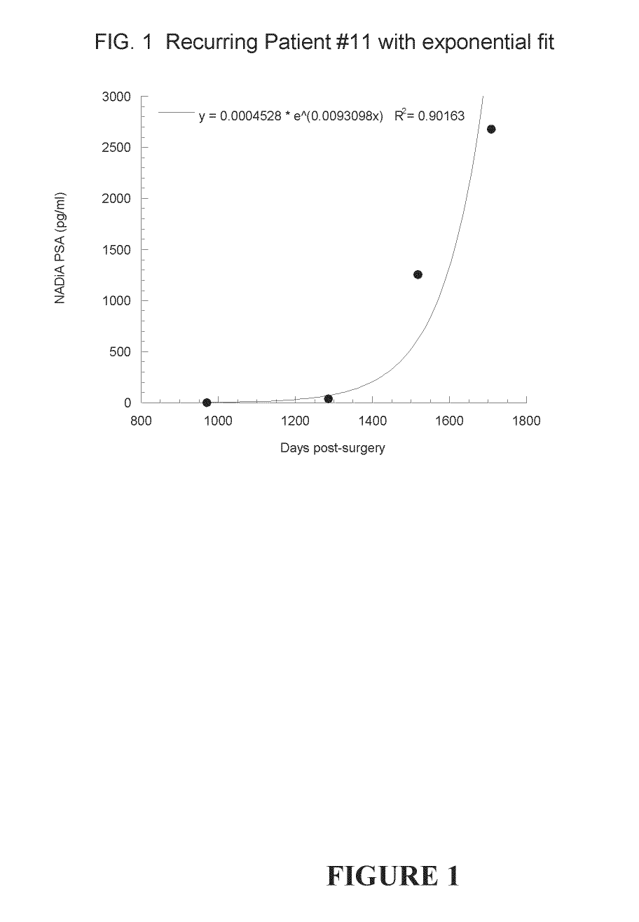

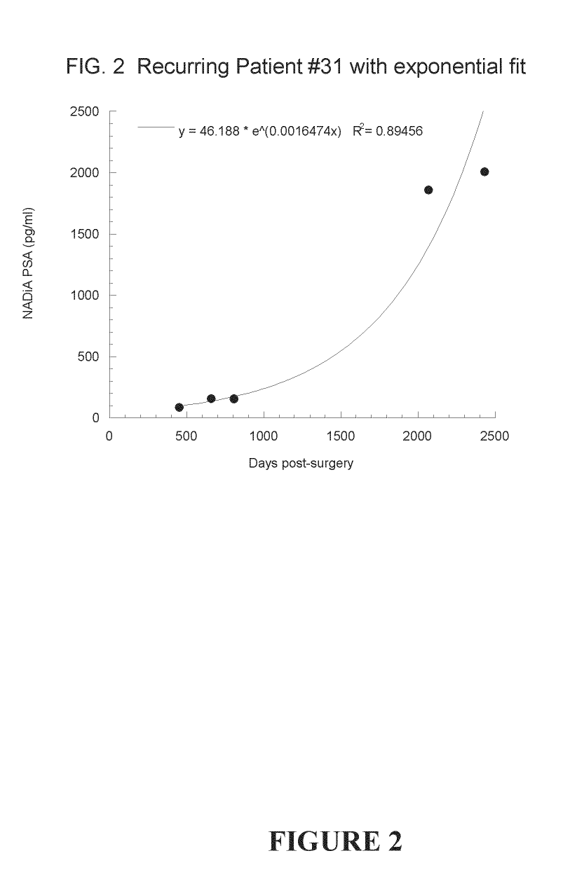

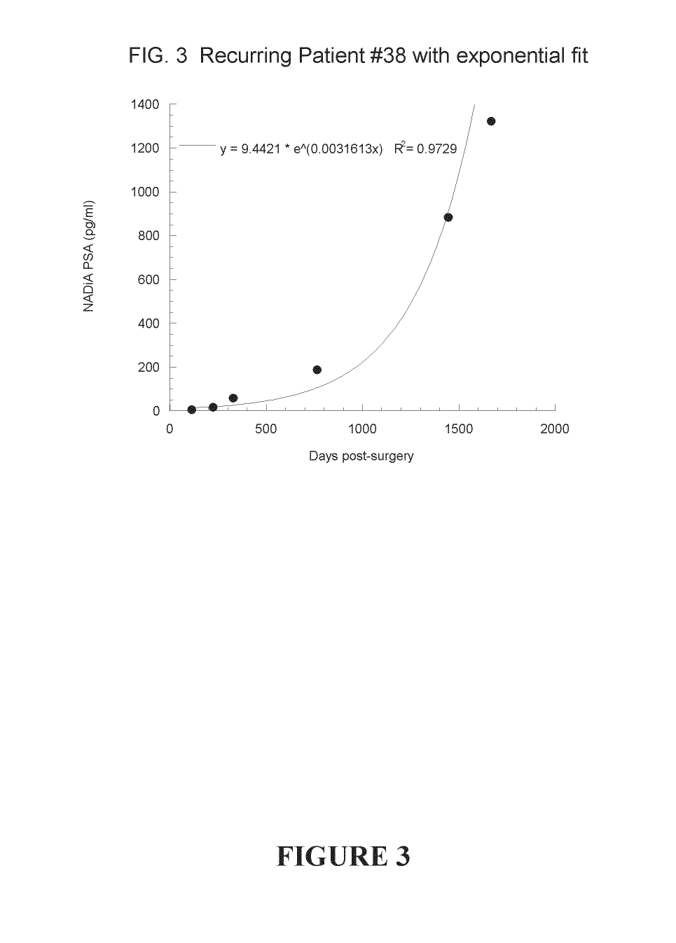

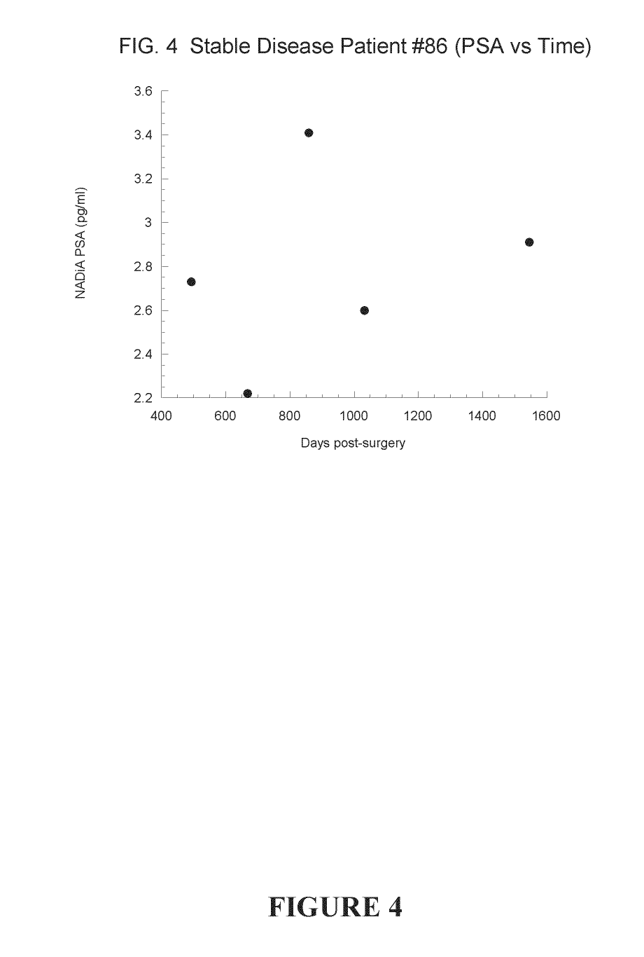

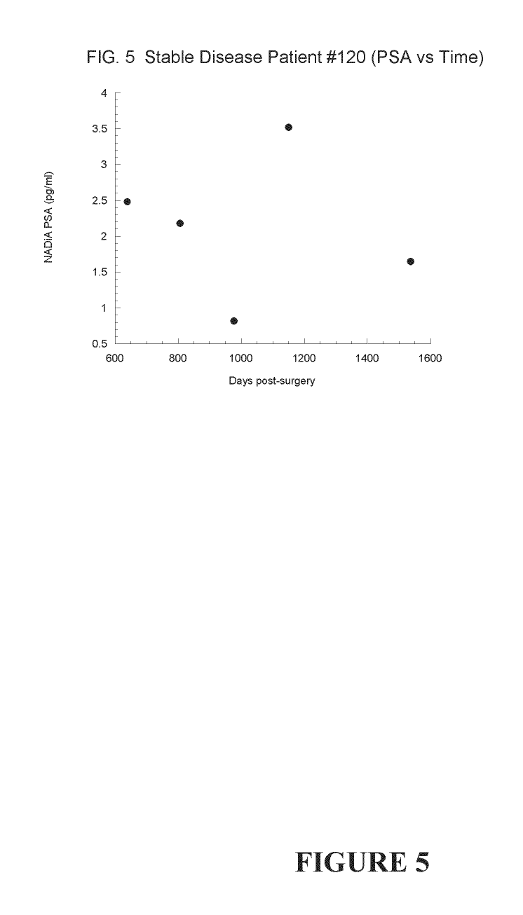

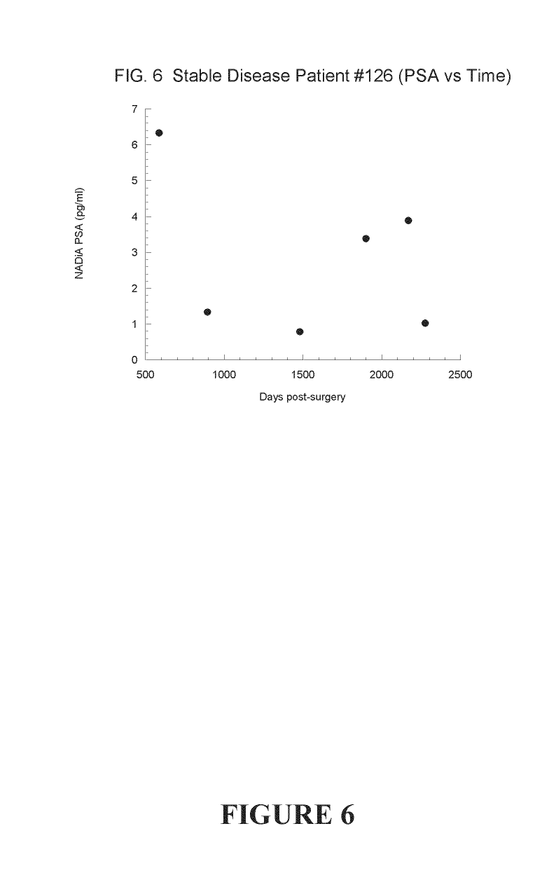

For each patient analyzed in this study, the tPSA (pg/mL) measured using ® assays was plotted as a function of days post-surgery. For example, FIG. 1 shows the plot of the ® t[PSA] in pg/mL vs. days post radical prostatectomy for recurring patient number 11, with exponential fit. FIG. 2 shows the plot of the ® t[PSA] in pg/mL vs. days post radical prostatectomy for recurring patient number 31, with exponential fit. FIG. 3 shows the plot of the ® t[PSA] in pg/mL vs. days post radical prostatectomy for recurring patient number 38, with exponential fit. FIG. 4 shows the plot of the ® t[PSA] in pg/mL vs. days post radical prostatectomy for stable patient number 86, with exponential fit. FIG. 5 shows the plot of the ® t[PSA] in pg/mL vs. days post radical prostatectomy for stable patient number 120, with exponential fit. FIG. 6 shows the plot of the ® [PSA] in pg/mL vs. days post radical prostatectomy for stable patient number 126, with exponential fit.

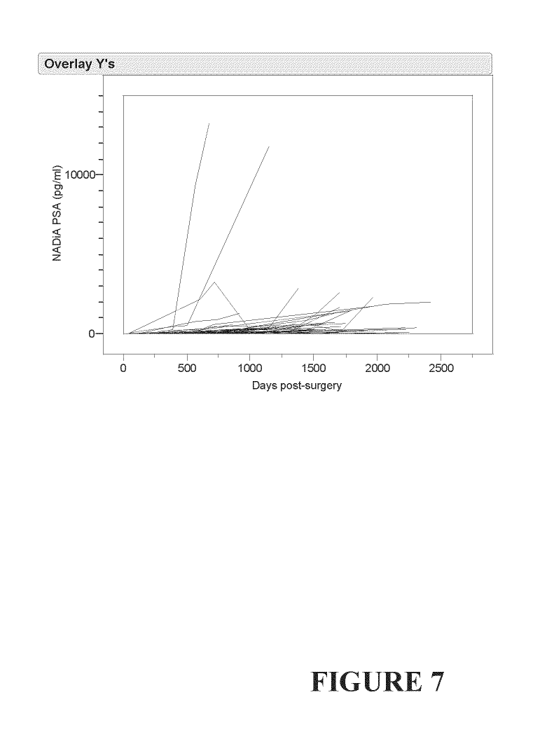

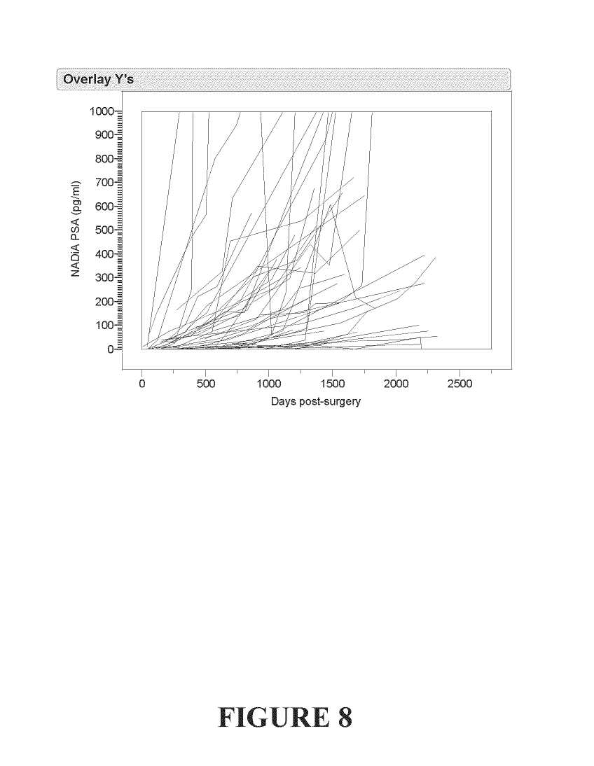

The plots for all patients were separated by whether patients fell into the Recurring category or Stable Disease category. FIG. 7 shows the plots of the ® t[PSA] in pg/mL vs. days post radical prostatectomy for all 43 recurring patients. FIG. 8 shows an overlay plot of the ® t[PSA] for 43 recurring patients vs time following prostatectomy, with range constrained to 1000 pg/ml, no points.

In the analysis of doubling time, the study excluded stable disease patients whose plots could not be fitted exponentially. Ten of the 42 stable disease patients were included in the doubling time analysis. For all other analyses (maximum observed PSA level, first post-prostatectomy PSA level, nadir PSA level, maximum observed PSA level/nadir level ratio, number of doublings, number of successive doublings, and 2^{nd} pg/mL/month rise) data from all 43 recurring and all 42 stable disease patients was utilized, i.e., no exclusions were made.

### Example 4

### Analyses of Potential Indices for Disease Outcome

An analysis of each possible PSA index (first post-prostatectomy PSA level, nadir PSA level, maximum observed PSA level, maximum observed PSA level/nadir level ratio, number of doublings, number of successive doublings, 2^{nd} pg/ml/month rise, doubling time (where exponential fits were possible)) versus recurring or stable disease was performed to assess the relative utility of each outcome as a predictor of recurrence. Clinical classification of patients as stable or having disease recurrence was used as a reference outcome. The statistical tests used were the Wilcoxon rank sum test for continuous variables, and the Pearson chi-square test for categorical variables.

The analyses demonstrated that all of the calculated [PSA] parameters were significant predictors (Wilcoxon rank sum or Pearson chi-square p<0.05) of clinical outcome (recurrence or stable disease). The maximum observed tPSA level, second consecutive increase pg/mL/month, and doubling time were the best at discriminating the patient sub-populations. The ratio of maximum PSA level to nadir level and the number of doublings also demonstrated fair discrimination.

The analysis for each of the [PSA] indices is discussed below.

### Example 4A

### Analysis of 1st Post-Prostatectomy Level vs. Patient Sub-Population (Recurrence or Stable Disease)

TABLE 4
Quantiles

| | | | | | | | Maxi- |
|---|---|---|---|---|---|---|---|
| Level | Minimum | 10% | 25% | Median | 75% | 90% | mum |
| 0 | 1.2 | 2.2 | 3.15 | 4.5 | 7.7 | 14.15 | 127 |
| 1 | 21.6 | 54.48 | 164 | 484.5 | 1406.4 | 2550.8 | 13316 |

TABLE 5
Means and Std Deviations

| | | | | Std Err | Lower | Upper |
|---|---|---|---|---|---|---|
| Level | Number | Mean | Std Dev | Mean | 95% | 95% |
| 0 | 40 | 8.96 | 19.67 | 3.11 | 2.67 | 15.3 |
| 1 | 43 | 1296.86 | 2648.59 | 403.91 | 481.75 | 2112.0 |

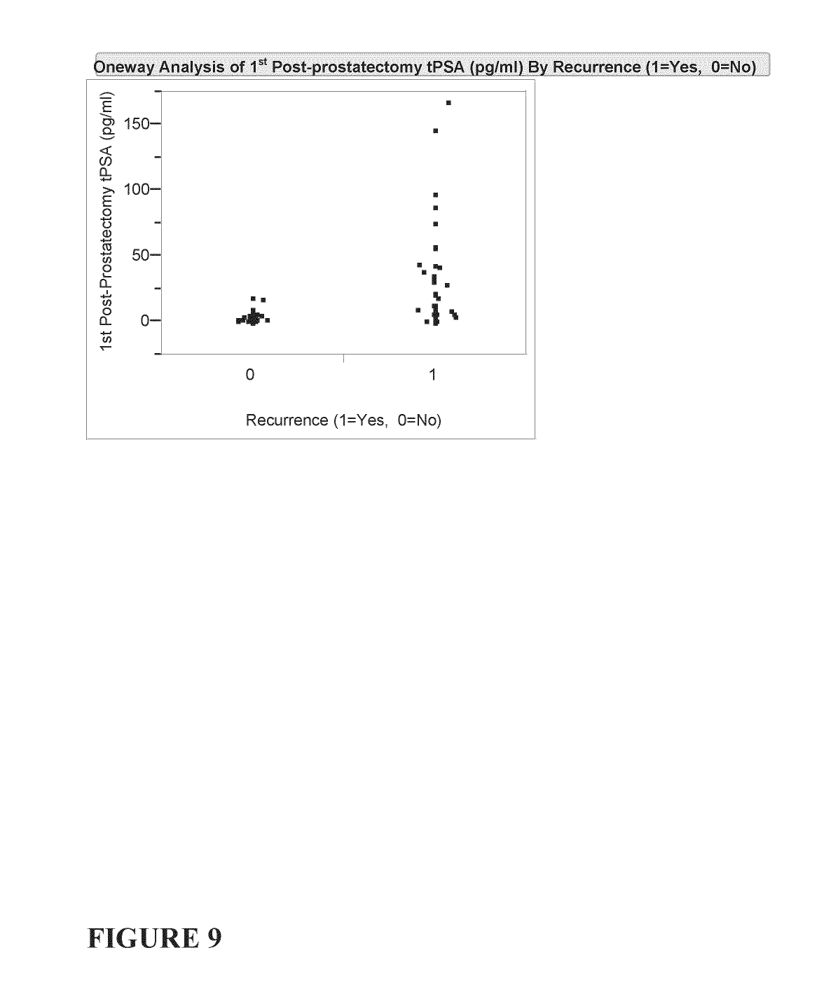

A plot of the first post-prostatectomy tPSA level vs. the patient sub-population (recurrence of prostate cancer (1) or with stable disease (0)) is shown in FIG. 9. Quintiles for the stable disease group (0) and the recurrence group (1) are shown in Table 4. The means and standard deviations for the stable disease group (0) and the recurrence group (1) are shown in Table 5. According to the data analysis for the plot of the first post-prostatectomy tPSA level vs. the patient sub-population (recurrence or stable disease), this parameter significantly differentiates the two populations and is thus a predictor of outcomes. The mean +/− standard error of the mean (SEM) [PSA] for the stable group was 4.1 pg/mL+/−0.58, while the mean+/−SEM [PSA] for the group having recurrence was 28.2+/−5.72. The p was <0.0001. However, the stable population overlaps the recurring population up to and beyond the median value.

### Example 4B

### Analysis of Nadir tPSA Level vs. Patient Sub-Population (Recurrence or Stable Disease)

TABLE 6
Quantiles

| | | | | | | | Maxi- |
|---|---|---|---|---|---|---|---|
| Level | Minimum | 10% | 25% | Median | 75% | 90% | mum |
| 0 | 0.2 | 0.8 | 0.975 | 1.7 | 2.95 | 4.38 | 17.4 |
| 1 | 0.4 | 1.48 | 4.7 | 9.7 | 39.1 | 83.16 | 167.9 |

TABLE 7
Means and Std Deviations

| | | | Std | Std Err | Lower | Upper |
|---|---|---|---|---|---|---|
| Level | Number | Mean | Dev | Mean | 95% | 95% |
| 0 | 42 | 2.3976 | 2.7038 | 0.4172 | 1.555 | 3.240 |
| 1 | 43 | 27.1605 | 37.7972 | 5.7640 | 15.528 | 38.793 |

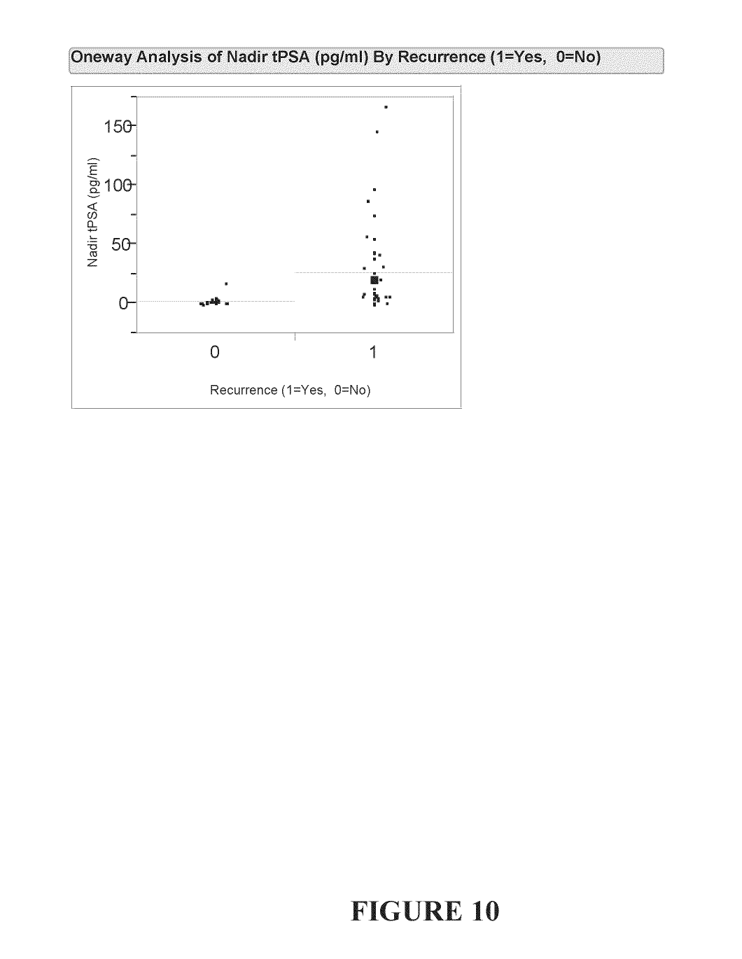

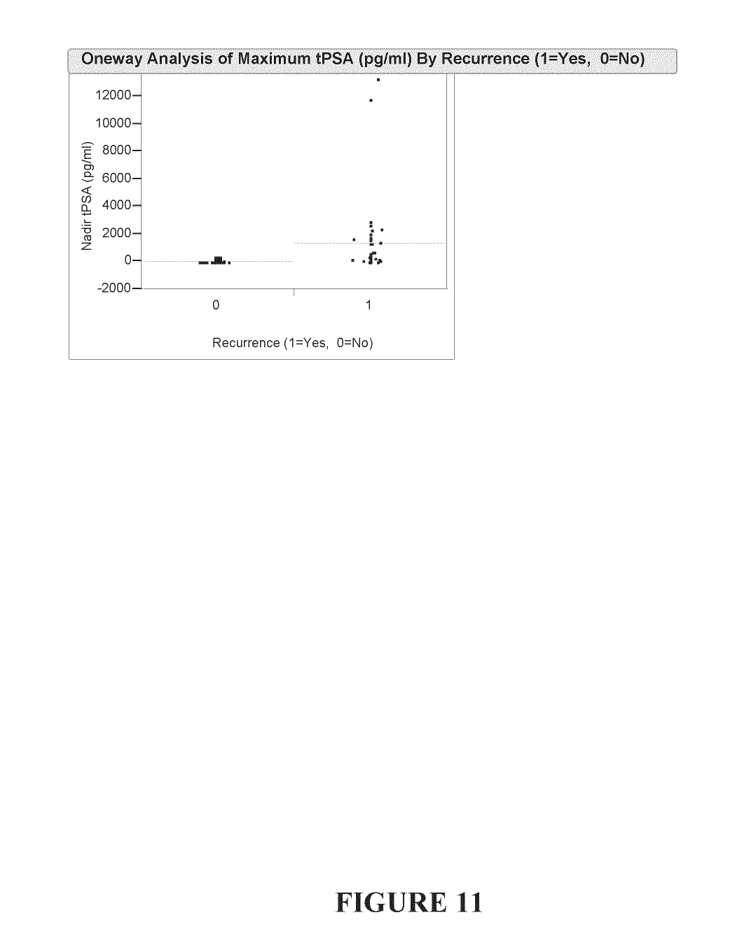

A plot of the nadir t[PSA] level (pg/mL) vs. the patient sub-population (recurrence of prostate cancer (1) or with stable disease (0)) is shown in FIG. 10. Quintiles for the stable disease group (0) and the recurrence group (1) are shown in Table 6. The means and standard deviations for the stable disease group (0) and the recurrence group (1) are shown in Table 7. According to the data analysis for the nadir [PSA] level, this parameter significantly differentiates the two populations and is thus a predictor of outcome. The mean+/−SEM nadir [PSA] for the stable group was 2.4 pg/mL+/−0.42, while the mean+/−SEM nadir [PSA] for the group having recurrence was 27.2+/−5.8. The p was <0.0001. However, the stable population overlaps the recurring population up to and beyond the median value.

### Example 4C

### Analysis of Maximum Observed tPSA Level vs. Patient Sub-Population (Recurrence or Stable Disease)

TABLE 8
Quantiles

| | Mini- | | | | | | Maxi- |
|---|---|---|---|---|---|---|---|
| Level | mum | 10% | 25% | Median | 75% | 90% | mum |
| 0 | 1.2 | 2.2 | 3.15 | 4.5 | 7.7 | 14.15 | 127 |
| 1 | 21.6 | 54.48 | 164 | 484.5 | 1406.4 | 2550.8 | 13316 |

TABLE 9
Means and Std Deviations

| | | | Std | Std Err | Lower | Upper |
|---|---|---|---|---|---|---|
| Level | Number | Mean | Dev | Mean | 95% | 95% |
| 0 | 40 | 8.96 | 19.67 | 3.11 | 2.67 | 15.3 |
| 1 | 43 | 1296.86 | 2648.59 | 403.91 | 481.75 | 2112.0 |

A plot of the maximum observed [PSA] level (pg/mL) vs. the patient sub-population (recurrence of prostate cancer (1) or with stable disease (0)) is shown in FIG. 11. Quantiles for the stable disease group (0) and the recurrence group (1) are shown in Table 8. The means and standard deviations for the stable disease group (0) and the recurrence group (1) are shown in Table 9. Analysis of the maximum observed [PSA] level vs. the patient sub-population showed that the maximum tPSA level significantly differentiated the two populations of stable and recurring patients and was therefore a predictor of outcome. The mean+/−SEM [PSA] for the stable group was 9.0 pg/mL+/−3.11, while the mean+/−SEM [PSA] for the group having recurrence was 1295.9+/−403.91. The p was <0.0001. The stable population only overlaps the recurring population somewhere between 10 and 25% and was thus nicely discriminated. In this study there was only one stable disease patient with an observed PSA level above 15 pg/mL.

### Example 4D

### Analysis of Maximum tPSA Level/Nadir Level vs. Patient Sub-Population (Recurrence or Stable Disease)

TABLE 10
Quantiles

| | Mini- | | | | | | Maxi- |
|---|---|---|---|---|---|---|---|
| Level | mum | 10% | 25% | Median | 75% | 90% | mum |
| 0 | 1.2 | 1.5 | 1.8 | 2.6 | 4.4 | 5.9 | 23.5 |
| 1 | 3.4 | 7.74 | 12 | 27.2 | 123 | 254.54 | 638.1 |

TABLE 11
Means and Std Deviations

| | | | Std | Std Err | Lower | Upper |
|---|---|---|---|---|---|---|
| Level | Number | Mean | Dev | Mean | 95% | 95% |
| 0 | 39 | 3.6154 | 3.602 | 0.577 | 2.448 | 4.78 |
| 1 | 43 | 87.5372 | 133.004 | 20.283 | 46.605 | 128.47 |

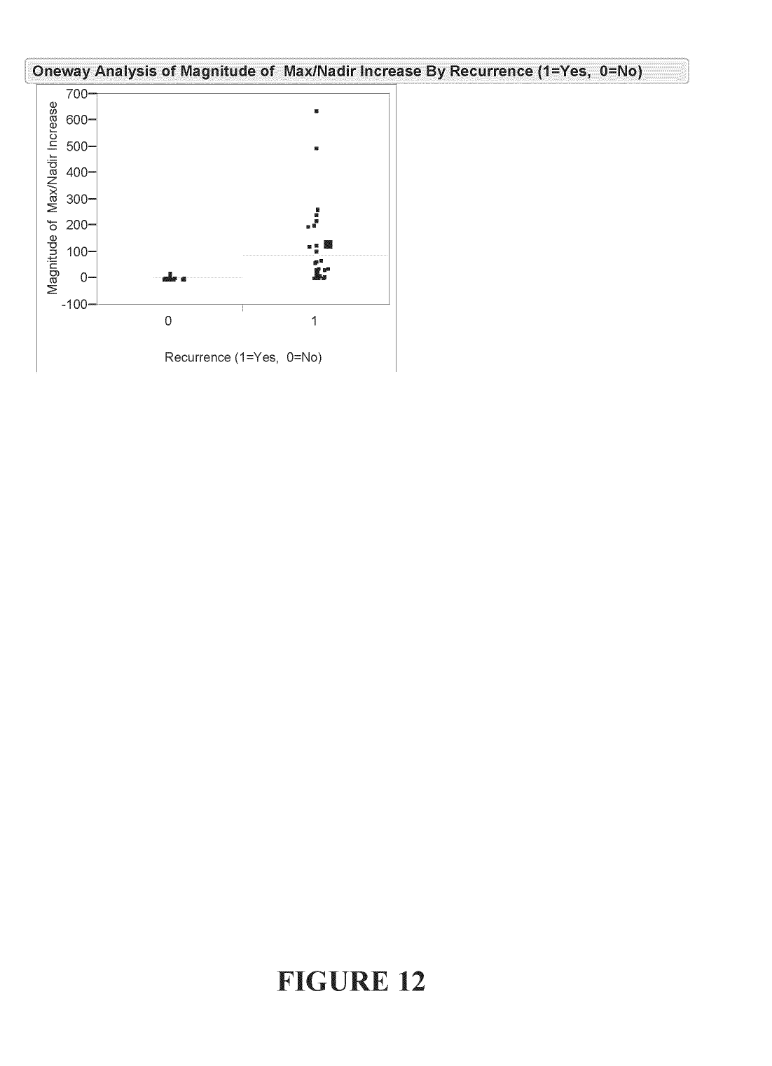

A plot of the maximum [PSA] level (pg/mL)/nadir level [PSA] (pg/mL) vs. the patient sub-population (recurrence of prostate cancer (1) or with stable disease (0)) is shown in FIG. 12. Quantiles for the stable disease group (0) and the recurrence group (1) are shown in Table 10. The means and standard deviations for the stable disease group (0) and the recurrence group (1) are shown in Table 11. Analysis of the maximum PSA level/nadir level PSA vs. patient sub-population showed that the ratio of the maximum PSA level to the nadir [PSA] significantly differentiates the two populations and is thus a predictor of outcome. The p was <0.001. The mean+/−SEM for the stable population was 3.6+/−0.6, while the mean+/−SEM for the recurring population was 87.5+/−20.3. However, the stable population overlaps the recurring population close to the median value.

### Example 4E

### Analysis of 2nd Consecutive Increase pg/mL/Month vs. Patient Sub-Population (Recurrence or Stable Disease)

TABLE 12
Quantiles

| | Mini- | | | | | | Maxi- |
|---|---|---|---|---|---|---|---|
| Level | mum | 10% | 25% | Median | 75% | 90% | mum |
| 0 | −0.73 | −0.195 | −0.085 | 0.015 | 0.175 | 0.332 | 5.4 |
| 1 | −140.7 | 1.64 | 4.7 | 7 | 20.1 | 117.36 | 1526.8 |

TABLE 13
Means and Std Deviations

| | | | Std | Std Err | Lower | Upper |
|---|---|---|---|---|---|---|
| Level | Number | Mean | Dev | Mean | 95% | 95% |
| 0 | 42 | 0.1490 | 0.861 | 0.133 | −0.12 | 0.42 |
| 1 | 43 | 63.4930 | 241.163 | 36.777 | −10.73 | 137.71 |

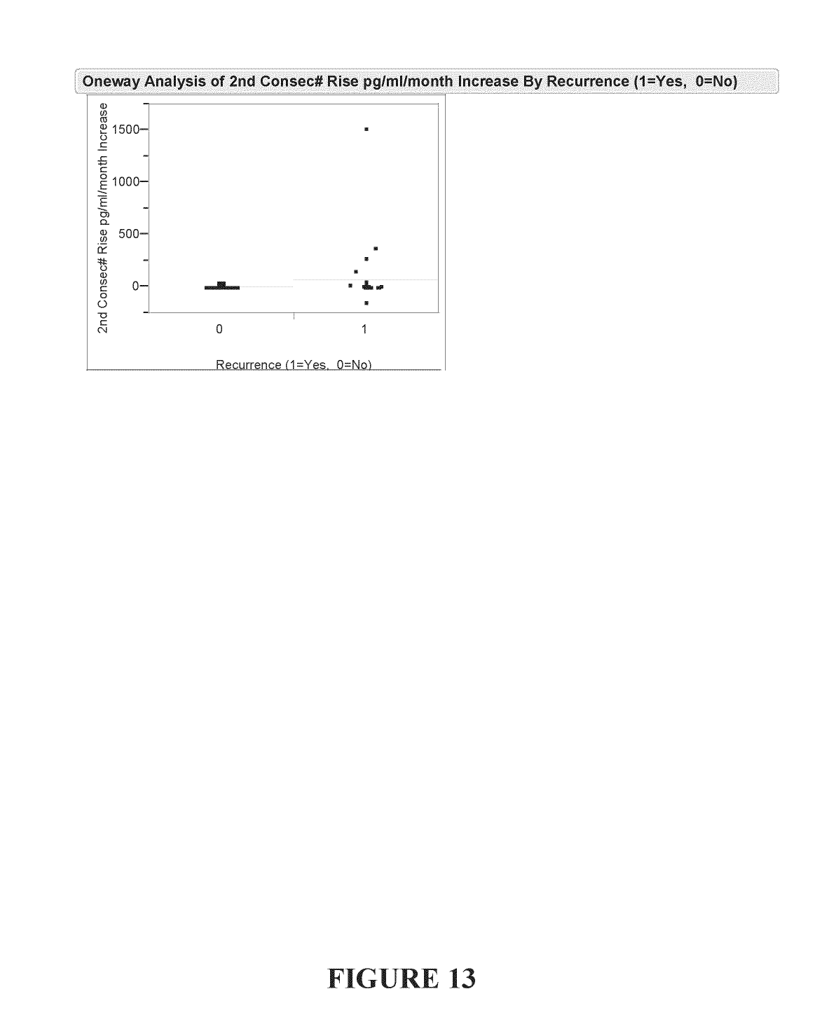

A plot of the second consecutive increase in [PSA] level (pg/mL/month) vs. the patient sub-population (recurrence of prostate cancer (1) or with stable disease (0) is shown in FIG. 13. Quantiles for the stable disease group (0) and the recurrence group (1) are shown in Table 12. The means and standard deviations for the stable disease group (0) and the recurrence group (1) are shown in Table 13. The analysis for the second consecutive increase (pg/mL/month) showed that this parameter significantly differentiates the two populations and is thus a predictor of outcome. The mean+/−SEM second consecutive increase for the stable group was 0.15 pg/mL/month+/−0.13, while the mean+/−SEM or the group having recurrence was 63.5+/−36.78. The p was <0.0001. The stable population overlaps the recurring population approximately 25% and thus indicates a good discriminatory power.

### Example 4F

### Analysis of Doubling Time (Days) vs. Patient Sub-Population (Recurrence or Stable Disease)

TABLE 14
Quantiles

| | Mini- | | | | | | Maxi- |
|---|---|---|---|---|---|---|---|
| Level | mum | 10% | 25% | Median | 75% | 90% | mum |
| 0 | 577.6 | 611.04 | 970.65 | 1127.7 | 1356.325 | 2127.22 | 2166.1 |
| 1 | 49.2 | 127.26 | 203.9 | 291.9 | 407.7 | 544.54 | 796.7 |

TABLE 15
Means and Std Deviations

| | | | Std | Std Err | Lower | Upper |
|---|---|---|---|---|---|---|
| Level | Number | Mean | Dev | Mean | 95% | 95% |
| 0 | 10 | 1207.99 | 451.736 | 142.85 | 884.84 | 1531.1 |
| 1 | 40 | 318.55 | 164.681 | 26.04 | 265.88 | 371.2 |

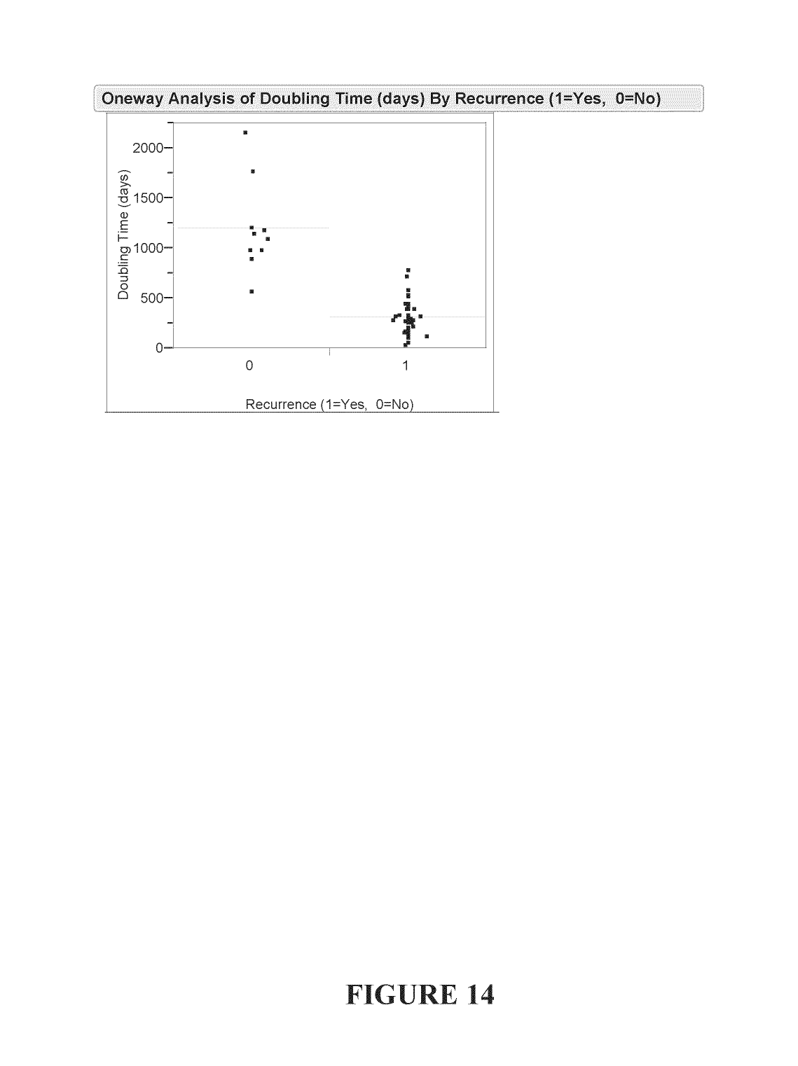

A plot of the doubling time data (days) vs. the patient sub-population (recurrence of prostate cancer (1) or with stable disease (0)) is shown in FIG. 14. Quintiles for the stable disease group (0) and the recurrence group (1) are shown in Table 14. The means and standard deviations for the stable disease group (0) and the recurrence group (1) are shown in Table 15. Analysis of the data showed that the doubling time (days) significantly differentiates the two populations and is thus a predictor of outcome. The p was <0.0001. The mean for the stable population was 1208+/142.9, while the mean for the recurring population was 318.6+/−26.04. The stable population only overlaps the recurring population between 10 and 25% and is thus nicely discriminated.

Additional Categorization of Patients Based on Doubling Time Observed Using a PSA Assay

Further analysis was undertaken to determine whether the doubling time could be used to discriminate between further subclasses of the recurring subpopulation of patients. The analysis of PSA doubling time permitted further sorting of patients into three groups, categorized by <150 days (rapid recurrences), 150-400 days (medium recurrences), and >400 days (slow recurrences). Rate was expected to reflect the rate of exponential growth, and therefore reflect the aggressiveness of the growth of the cancer.

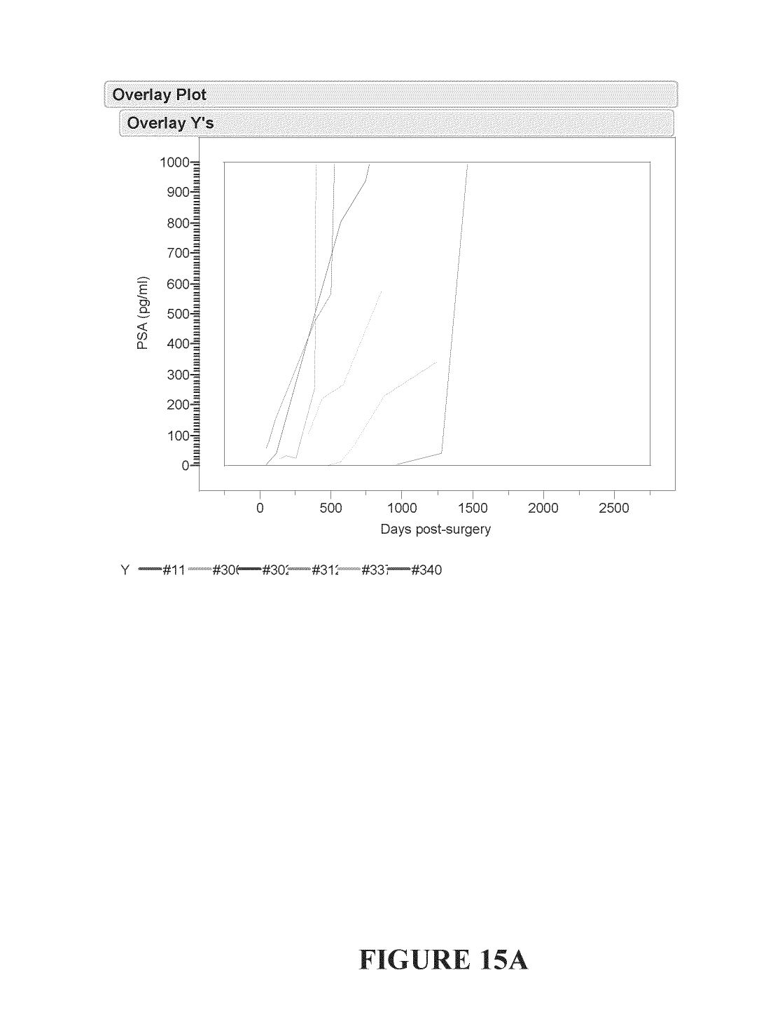

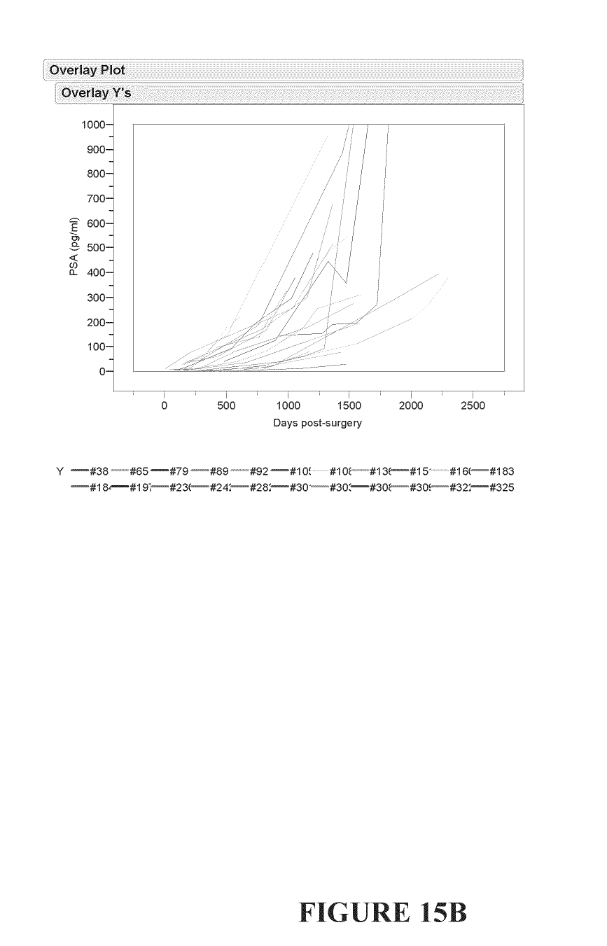

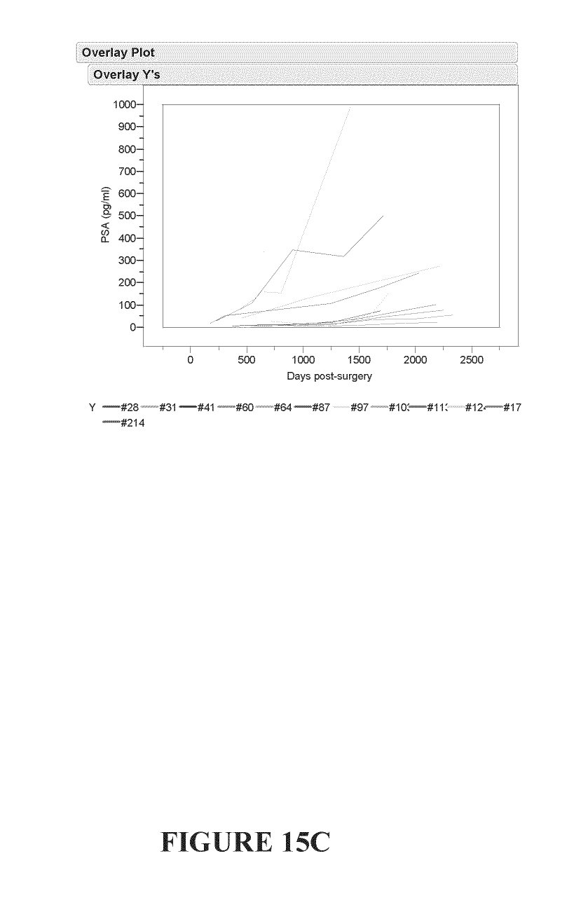

FIGS. 15A-C show the overlay plots for recurring patients with doubling times of <150 days, 150-400 days, or >400 days, respectively. FIG. 15A shows the overlay plots for recurring patients, of [PSA] pg/ml vs days post surgery, with doubling times of <150 with range constrained to 1000 pg/mL

FIG. 15B shows the overlay plots for recurring patients, of [PSA] pg/ml vs days post surgery with doubling times of 150-400 with range constrained to 1000 pg/mL

FIG. 15C shows the overlay plots for recurring, of [PSA] pg/ml vs days post surgery patients with doubling times of >400 with range constrained to 1000 pg/Ml

The recurring patients can be divided into four classes, Group 1, doubling time of less than 150 days, Group 2, with doubling times between 150-400 days, Groups 3 and 4, which both had doubling times greater than 400 days. In Group 3 the maximum observed PSA exceeded 200 pg/mL, while in Group 4 the maximum observed PSA did not exceed 200 pg/mL.

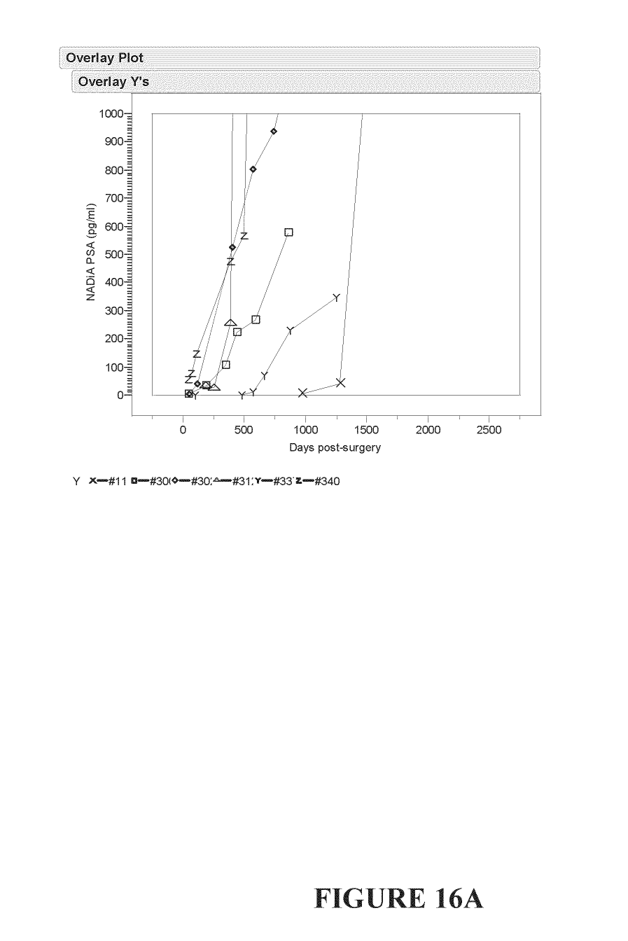

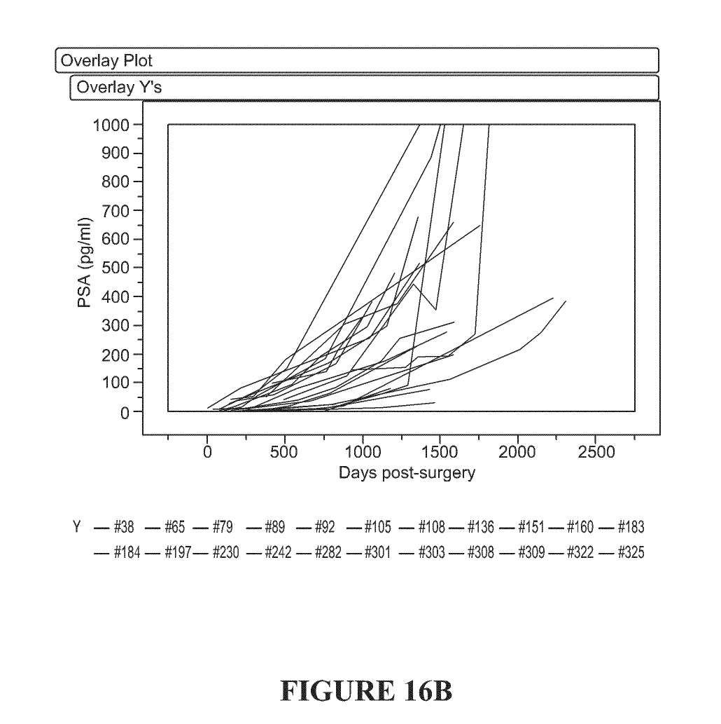

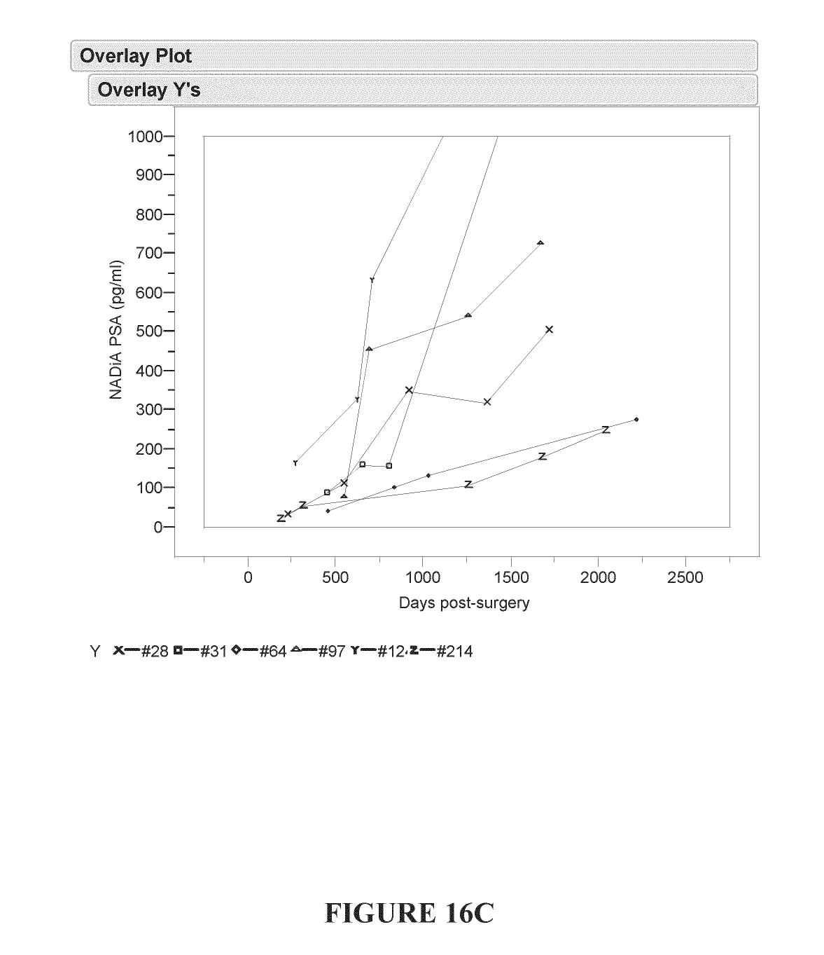

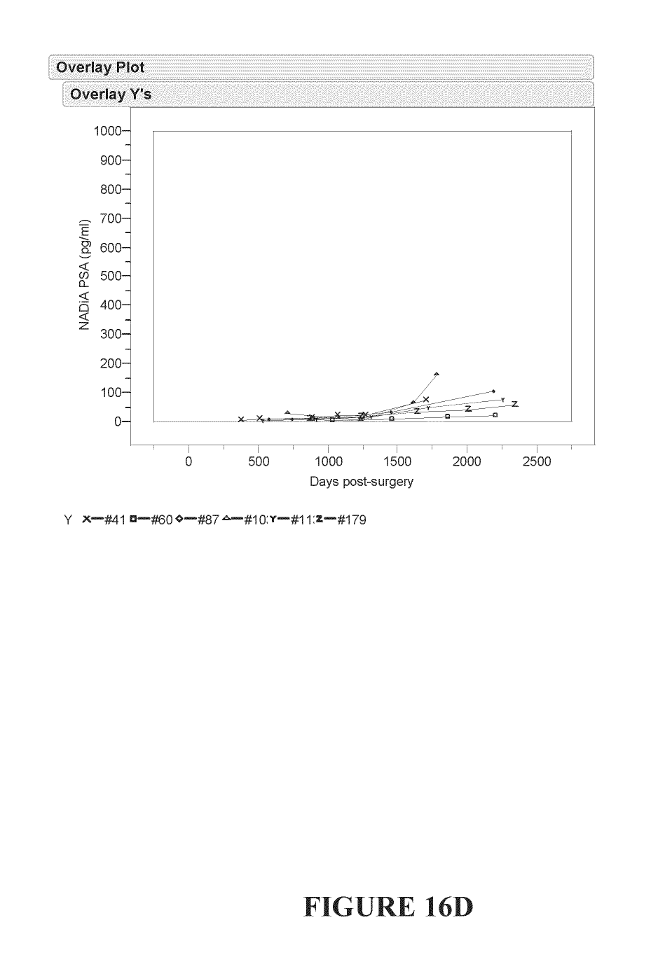

FIGS. 16A-D shows the overlay plots for subclasses of recurring patients by doubling time, with ranges constrained to 1000 pg/mL, respectively. The recurring patients with doubling times of >400 days have been further subdivided whether the maximum observed PSA is above or below 200 pg/mL.

FIG. 16A shows the overlay plots for recurring patients with doubling time <150 days of [PSA] pg/ml vs days post surgery.

FIG. 16B shows the overlay plots for recurring patients with doubling time <150-400 days of [PSA] pg/ml vs days post surgery.

FIG. 16C shows the overlay plots for recurring patients with doubling time >400 days, maximum [PSA] >200 pg/mL vs days post surgery.

FIG. 16D shows the overlay plots for recurring patients [PSA] pg/ml vs days post surgery.

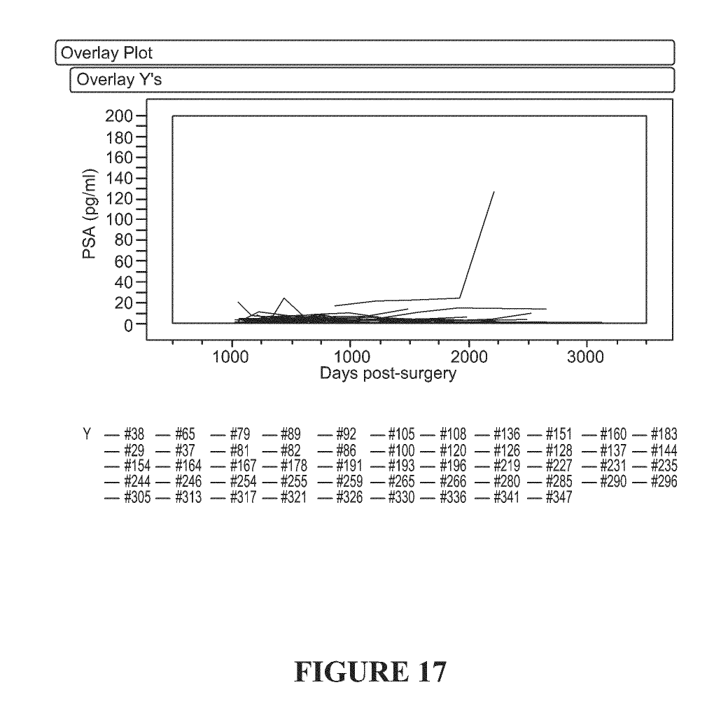

FIG. 17 shows the overlay plots of [PSA] pg/ml vs days post surgery that, with few exceptions, the stable disease patients generally have PSA maximums which do not exceed 15 pg/mL.

### Example 4G

### Univariate Analysis of Number of Doublins vs. Patient Sub-Population (Recurrence or Stable Disease)

TABLE 16
Contingency Table

| | Count | | | | | | |
|---|---|---|---|---|---|---|---|
| | Total % | | | | | | |
| | Col % | # of Doublings During Monitoring | | | | | |
| | Row % | 0 | 1 | 2 | 3 | 4 | |
| Recurrence | 0 | 15 | 23 | 4 | 0 | 0 | 42 |
| (1 = Yes, | | 17.65 | 27.06 | 4.71 | 0.00 | 0.00 | 49.41 |
| 0 = No) | | 100.00 | 71.88 | 17.39 | 0.00 | 0.00 | |
| | | 35.71 | 54.76 | 9.52 | 0.00 | 0.00 | |
| | 1 | 0 | 9 | 19 | 12 | 3 | 43 |
| | | 0.00 | 10.59 | 22.35 | 14.12 | 3.53 | 50.59 |
| | | 0.00 | 28.13 | 82.61 | 100.00 | 100.00 | |
| | | 0.00 | 20.93 | 44.19 | 27.91 | 6.98 | |
| | | 15 | 32 | 23 | 12 | 3 | 85 |
| | | 17.65 | 37.65 | 27.06 | 14.12 | 3.53 | |

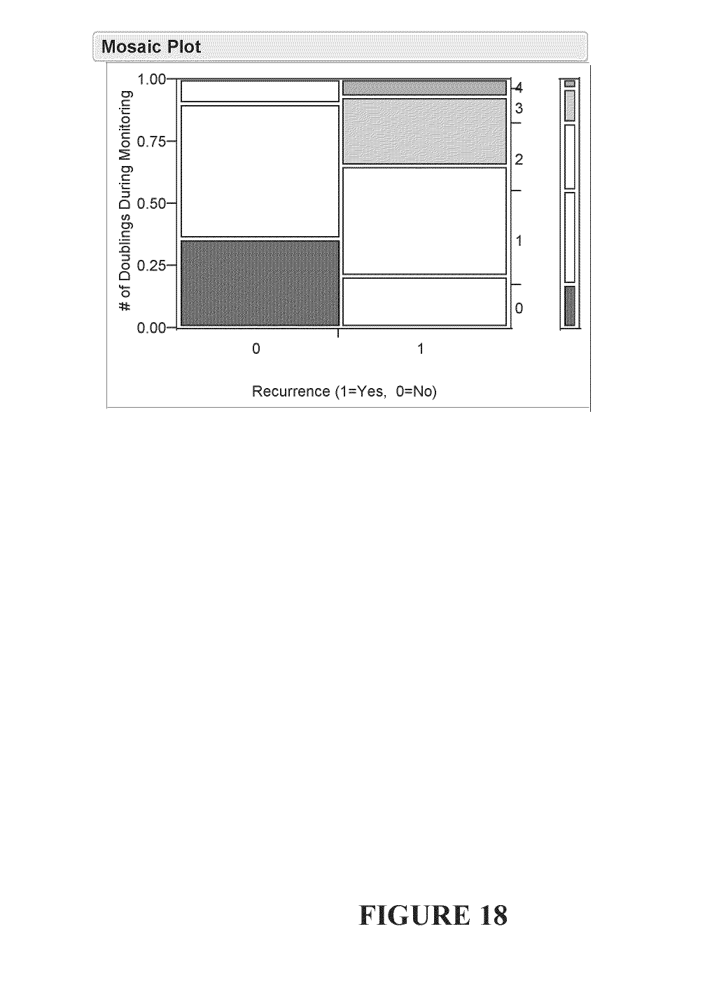

Table 16 above demonstrates that the number of doublings is increased for the 43 patients with prostate cancer recurrence versus the 42 patients with stable disease. The difference was significant at p<0.0001 (Chi-square). There is some overlap between sub-populations in the areas of 1 and 2 doublings. The degree of overlap is approximately 60% of the overall population, but it is of interest that (a) a doubling is always observed for recurrence and (b) there are no patients with 3 or 4 doublings in stable disease. A mosaic plot of the data showing the number of doublings during monitoring vs. the patient subpopulation of recurrence of prostate cancer (1) or with stable disease (0) is shown in FIG. 18.

### Example 4H

### Univariate Analysis of Number of Consecutive Doublings vs. Patient Sub-Population (Recurrence or Stable Disease)

TABLE 17
Contingency Table

| | Count | | | | | | |
|---|---|---|---|---|---|---|---|
| | Total % | | | | | | |
| | Col % | # of Successive Doublings | | | | | |
| | Row % | 0 | 1 | 2 | 3 | 4 | |
| Recurrence | 0 | 40 | 0 | 2 | 0 | 0 | 42 |
| (1 = Yes, | | 47.06 | 0.00 | 2.35 | 0.00 | 0.00 | 49.41 |
| 0 = No) | | 74.07 | 0.00 | 10.00 | 0.00 | 0.00 | |
| | | 95.24 | 0.00 | 4.76 | 0.00 | 0.00 | |
| | 1 | 14 | 4 | 18 | 6 | 1 | 43 |
| | | 16.47 | 4.71 | 21.18 | 7.06 | 1.18 | 50.59 |
| | | 25.93 | 100.00 | 90.00 | 100.00 | 100.00 | |
| | | 32.56 | 9.30 | 41.86 | 13.95 | 2.33 | |
| | | 54 | 4 | 20 | 6 | 1 | 85 |
| | | 63.53 | 4.71 | 23.53 | 7.06 | 1.18 | |

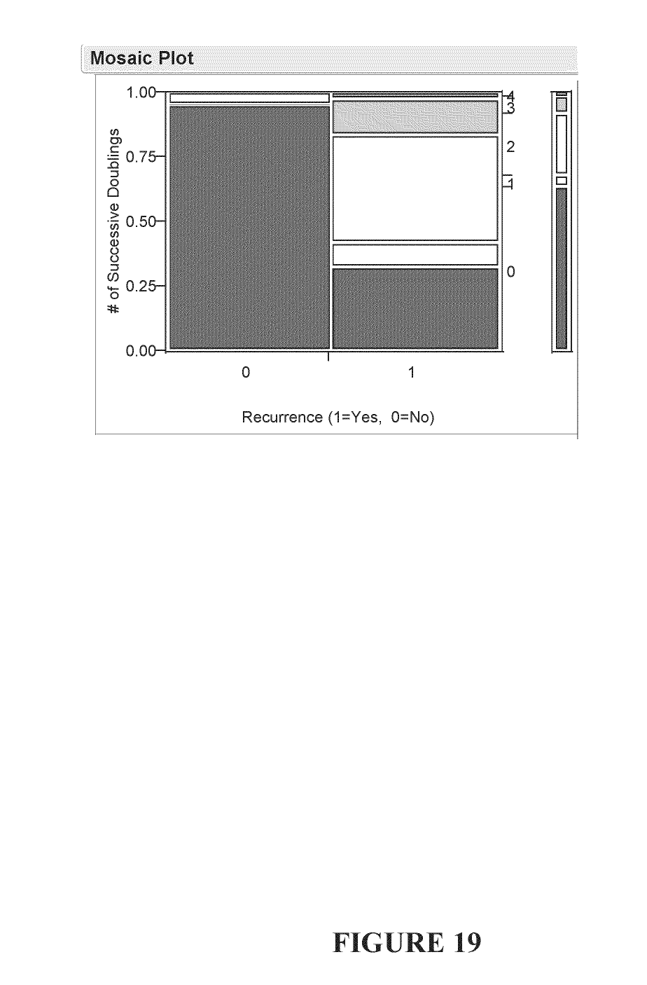

Table 17 above demonstrates that the number of consecutive doublings is increased in the 43 patients with prostate cancer recurrence vs. the 42 patients with stable disease. The difference was significant at p<0.0001 (Chi-square). The degree of overlap is approximately 80% of the overall population. A mosaic plot of the data showing the number of consecutive doublings vs. the patient subpopulation of recurrence of prostate cancer (1) or with stable disease (0) is shown in FIG. 19.

### Example 5

### Index Evaluation Using Univariate Logistic Regression and Receiver-Operating Characteristic (ROC) Analysis

Univariate logistic regression and receiver operating characteristic (ROC) curve analyses were used in evaluating whether various indices based on PSA measurements (first post-prostatectomy PSA level, nadir PSA level, maximum observed PSA level, number of doublings, number of successive doublings, 2^{nd} pg/mL/month rise) were predictive of disease outcome. The clinical classification of patients as stable or having recurring disease was used as a reference. Additionally, for calculation of doubling time, statistical analysis showed that exponential and other fits were appropriate for 40 of 43 recurring patients and 10 of 42 stable disease patients. Exponential parameters were taken for doubling time calculations if R2 was at least ˜0.5, even if other fits gave a better fit. In addition, the tPSA values must have been rising with time for calculation of doubling time.

To assess the ability of candidate ® assay indices to predict biochemical recurrence of prostate cancer, logistic regression and ROC analyses were employed. Logistic regression models taking each candidate index separately (in its own model) including maximum observed value, doubling time, maximum observed PSA lev1/nadir level ratio, 2^{nd} pg/mL/month, and number of doublings, were used to generate Odds Ratios (a measure of treatment effect that compares the probability of a type of outcome in the treatment group with the outcome of a control group; odds ratios deviating significantly from a value of 1.0 are desired) and p-values from the Wald test. ROC analysis provided point estimates of the area under the ROC curve (plotted as Sensitivity vs. 100-Specificity; an area of 1.0 is ideal) and the associated 95% confidence intervals (95% CIs), the best discriminating index value, and the associated Sensitivity and Specificity at the best discriminating index value. The results are summarized in Tables 18 and 19, below.

Summary of Results of Univariate Analyses

TABLE 18
Summary of Univariate Logistic Regression and ROC Results:

| Parameter | AUC | Wald p |
|---|---|---|
| Maximum | 0.994 | 0.0009 |
| observed value | | |
| Doubling time | 0.992 | |
| Max/Nadir | 0.973 | 0.0002 |
| pg/mL/month | 0.968 | 0.0444 |
| Number | 0.902 | |
| doublings | | |

TABLE 19
Summary of Univariate Logistic Regression and ROC Results

| | Parameter | | | | | | |
|---|---|---|---|---|---|---|---|
| | | | | | | | Sensitivity/ |
| | Odds | Wald | | AUC | Discriminating | | Specificity at |
| | Ratio | p-value | ROC-AUC | 95% Cl | Cutpoint | | cutpoint |
| Doubling time | | | 0.992 | 0.914-1.000 | 545.8 | days | 93%/100% |
| Maximum | 1.0657 | 0.0009 | 0.994 | 0.994-0.996 | 25.1 | pg/mL/mo | 98%/98% |
| observed value | | | | | | | |
| Max/Nadir | 1.4718 | 0.0002 | 0.973 | 0.911-0.996 | 6.1 | | 95%/95% |
| ratio | | | | | | | |
| 2^{nd} Rise | 1.0516 | 0.0444 | 0.968 | 0.905-0.994 | 0.6 | pg/mL/mo | 95%//98% |
| pg/mL/month | | | | | | | |

| | | | | |
|---|---|---|---|---|
| # Doublings | 0.902 | 0.818-0.956 | 1 | 79%/90% |

Areas under the ROC curves were close to the ideal state of 1.0 and the combinations of sensitivity and specificity were high except for the index of # of doublings. The logistic regression models for doubling time and # of doublings failed to converge due to limitations of observations. Thus, the strongest indices of sub-populations (stable disease and early stage biochemical recurrence) were maximum observed level, the maximum PSA level/nadir level ratio, and the 2^{nd} pg/mL/month increase in ® assay PSA levels. All these indices were significant predictors of biochemical recurrence (all Wald p values were <0.05).

### Example 6

### Index Evaluation Using Multivariate Logistic Regression and ROC

To further assess the candidate indices found to be strong predictors in univariate analysis (maximum observed level, maximum level/nadir ratio, and 2^{nd} pg/mL/month increase in ® assay PSA), multivariate logistic regression and ROC analyses were performed. The intent was to determine if the ® assay candidate indices were able to maintain predictive capability even in the presence of clinicopathological prognostic indices within the models. These clinicopathological indices all had been previously shown to be significant predictors of recurrence and included: surgical margin involvement; capsular invasion of cancer; and peri-prostatic tissue invasion of cancer.

For each model, Odds Ratio and Wald p-value are provided for the ® assay index and the clinicopathological indices. The overall area under the curve (AUC) of the ROC and it's associated 95% CI are also presented. Additionally, the significance of the difference between the AUC for the multivariate model vs. the AUC for the univariate model of the ® assay index was determined statistically. If the p-value for this statistical interpretation was <0.05 it would indicate that the multivariate model displayed increased predictive power over the ® assay index by itself and conversely p-values >0.05 would indicate that the ® assay index is a powerful and independent predictor and that adding clinicopathological indices to the model does not significantly improve predictive capability for detection of prostate cancer recurrence.

The following figures and tables present the multivariate ROC curves in comparison to the univariate ROC curves employing the ® assay index only, and the results of the logistic regression and ROC computations.

### Example 6A

### Multivariate Results—Maximum Observed PSA

TABLE 20

| | | | | | p vs. |
|---|---|---|---|---|---|
| | Odds | Wald | ROC- | AUC | Max |
| Term | Ratio | p-value | AUC | 95% CI | by itself |
| NADIA Maximum | 1.066 | 0.0497 | 0.996 | 0.918-1.000 | 0.797 |
| Surgical margins | 236.3 | 0.0962 | | | |
| (categorical) | | | | | |
| Peri-Prost Tissue | 19.5 | 0.7478 | | | |
| invasion | | | | | |
| (categorical) | | | | | |
| Capsular invasion | 0.0042 | 0.5700 | | | |
| (categorical) | | | | | |

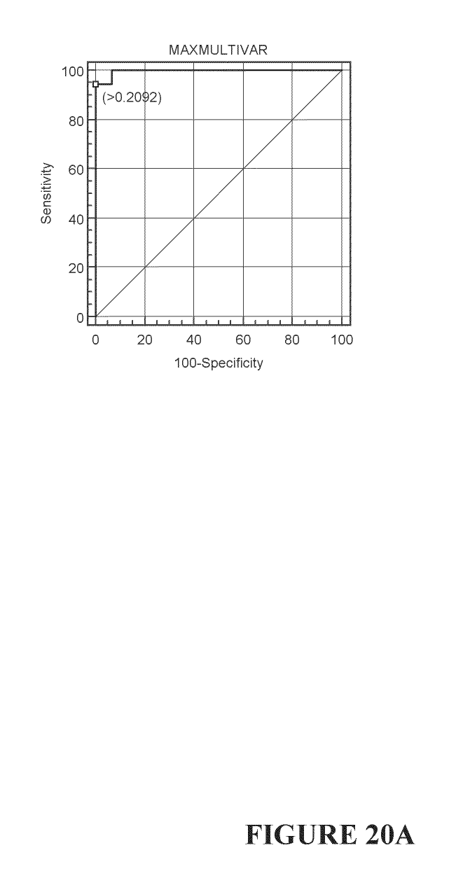

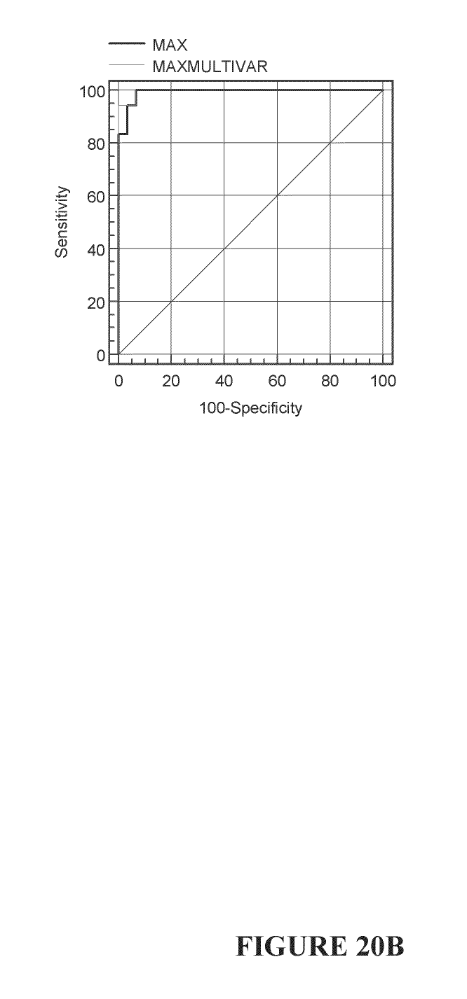

FIGS. 20A and 20B show the multivariate ROC curve in comparison to the univariate ROC curve for the ® maximum observed [PSA] level. FIG. 20A shows the multivariate ROC curve. FIG. 20B shows the univariate ROC curve for the ® maximum observed [PSA] level (black line) vs. the multivariate ROC curve (dotted line). Table 20 shows the results of the logistic regression and ROC computations. A logistic regression model for maximum observed [PSA] value was used to generate Odds Ratios and p-values from the Wald test. ROC analysis provided point estimates of the area under the curve (AUC) and it's associated 95% CI are also presented.

The ® maximum observed PSA level is an independent and significant predictor of outcome (p=0.0497) and the multivariate model does not significantly improve AUC (p=0.797) compared to using the parameter by itself.

### Example 6B

### Multivariate Results—Maximum tPSA/Nadir Ratio

TABLE 21

| | Regression | | Odds | Wald | | AUC | p vs. Max/ |
|---|---|---|---|---|---|---|---|
| Term | Coeff. | SE | Ratio | p-value | ROC-AUC | 95% CI | Nadir by itself |
| NADIA Max/Nadir ratio | 0.2764 | 0.098 | 1.3184 | 0.0051 | 0.963 | 0.866-0.995 | 0.191 |
| Surgical margins (categorical) | 1.7221 | 1.04 | 5.5964 | 0.0982 | | | |
| Peri-Prost Tissue invasion (categorical) | 1.0151 | 1.28 | 2.7597 | 0.4277 | | | |
| Capsular invasion (categorical) | 1.1495 | 2.03 | 3.1567 | 0.5711 | | | |

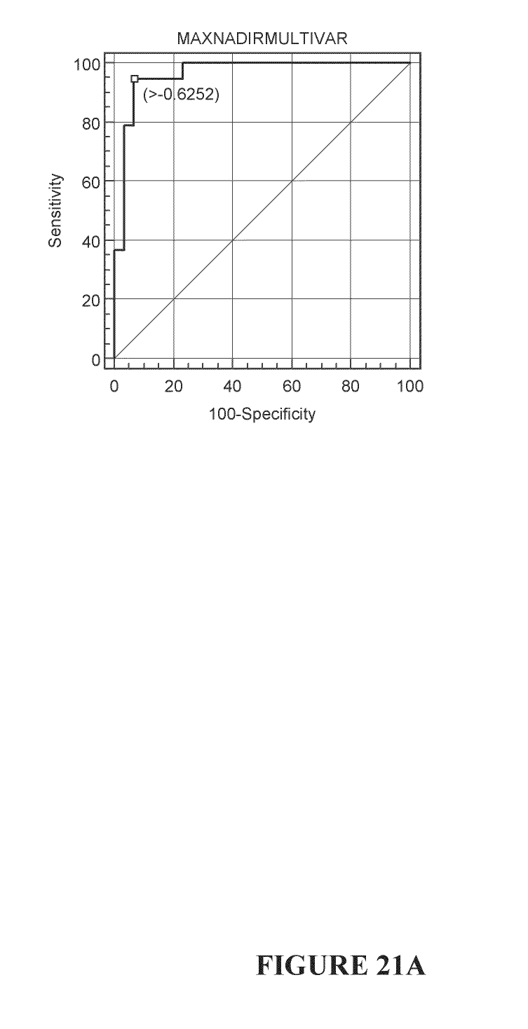

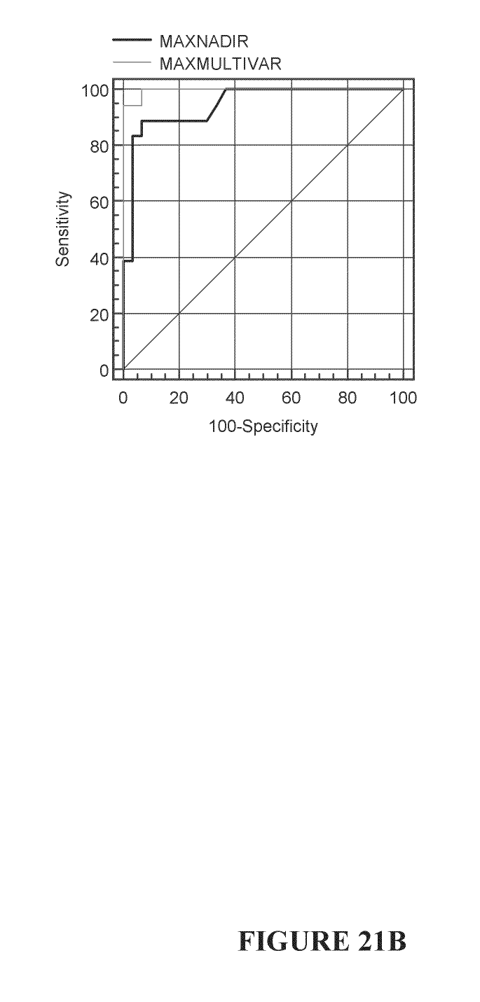

FIGS. 21A and 21B show the multivariate ROC curve in comparison to the univariate ROC curve for the ® maximum total [PSA]/nadir [PSA] levels. FIG. 21A shows the multivariate ROC curve. FIG. 21B shows the univariate ROC curve for the ® maximum total [PSA]/nadir [PSA] levels (black line) vs. the multivariate ROC curve (dotted line). Table 21 shows the results of the logistic regression and ROC computations. A logistic regression models for maximum total [PSA]/nadir [PSA] levels was used to generate Odds Ratios and p-values from the Wald test. ROC analysis provided point estimates of the area under the curve (AUC) and it's associated 95% CI are also presented.

The ratio of the maximum observed PSA level to the nadir PSA level is an independent predictor of outcome (p=0.0051) and the multivariate model does not significantly improve AUC (p=0.191) compared to using maximum observed PSA/nadir by itself.

### Example 6C

### Multivariate Results—Second Rise (pg/mL/Month)

TABLE 22

| | | | | | p |
|---|---|---|---|---|---|
| | | Wald | ROC- | AUC | vs. pg/ml/mo |
| Term | Odds Ratio | p-value | AUC | 95% CI | by itself |
| NADIA | 4.4250 | 0.0023 | 0.991 | 0.924-0.995 | 0.701 |
| pg/ml/month | | | | | |
| Surgical | 16.1609 | 0.0553 | | | |
| margins | | | | | |
| (categorical) | | | | | |

Model did not converge when Peri-Prost Tissue Invasion and Capsular Invasion were included.

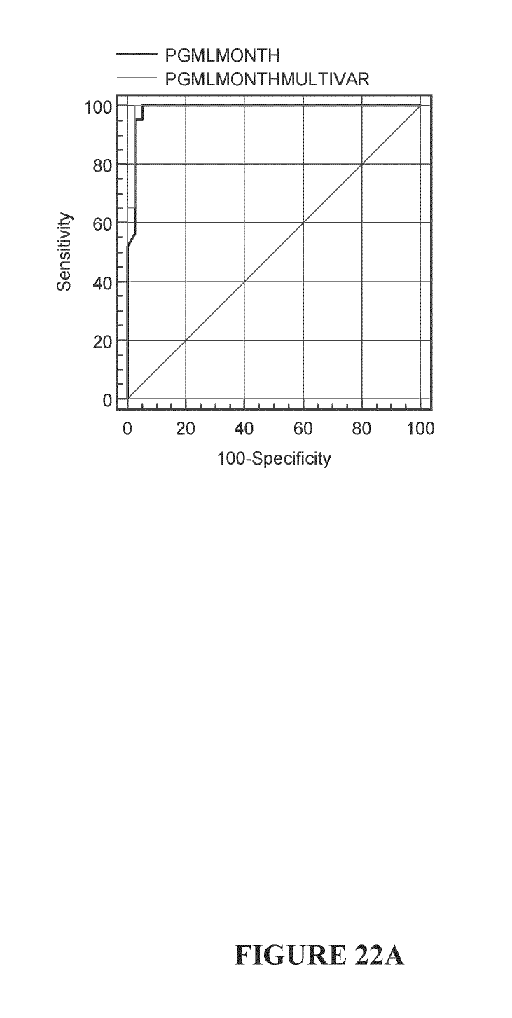

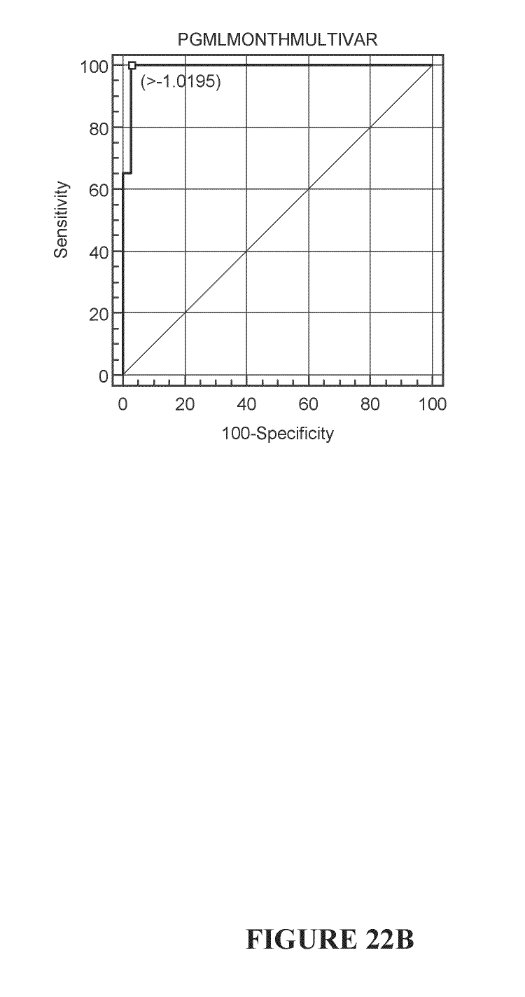

FIGS. 22A and 22B show the multivariate ROC curve in comparison to the univariate ROC curve for the second rise in [PSA] (pg/mL/month). FIG. 22B shows the multivariate ROC curve. FIG. 22A shows the univariate ROC curve for the ® second rise in [PSA] (pg/mL/month) (black line) vs. the multivariate ROC curve (dotted line). Table 22 shows the results of the logistic regression and ROC computations. A logistic regression models for second rise in [PSA] (pg/mL/month) was used to generate Odds Ratios and p-values from the Wald test. ROC analysis provided point estimates of the area under the curve (AUC) and it's associated 95% CI are also presented.

The second rise (pg/mL/month) is an independent predictor of outcome (p=0.0023) and the multivariate model does not significantly improve AUC (p=0.701) compared to using the second rise by itself.

### Example 7

### Evaluation of [PSA] Indices as Binary Categorical Representations

Univariate logistic regression and ROC analyses to evaluate the use of maximum observed PSA level, the maximum level/nadir level ratio, and the second rise (pg/mL/month) as binary categorical representations was also performed. Results are shown in Table 23. Index cutoffs for the binary categorical representations were 25 pg/mL maximum observed level, 0.6 pg/mL/month value for second rise and a maximum observed PSA level/nadir level ratio of 6. Each patient was categorized as either exceeding or not exceeding these cutoffs.

TABLE 23
BINARY CATEGORICAL REPRESENTATIONS

| | | | | | | |
|---|---|---|---|---|---|---|
| | Regression | | Odds | Wald | | AUC |
| Term | Coeff. | SE | Ratio | p-value | ROC-AUC | 95% CI |

Univariate Logistic Regression for: Maximum observed value post-prostatectomy (Binary)

| | | | | | | |
|---|---|---|---|---|---|---|
| NADIA Maximum | −6.6821 | 1.25 | 0.0013 | <0.0001 | 0.963 | 0.897-0.992 |

Univariate Logistic Regression for: Max/Nadir (Binary)

| | | | | | | |
|---|---|---|---|---|---|---|
| NADIA Max/Nadir ratio | −5.5053 | 0.94 | 0.0041 | <0.0001 | 0.938 | 0.862-0.979 |

Univariate Logistic Regression for: pg/ml/month (Binary)

| | | | | | | |
|---|---|---|---|---|---|---|
| NADIA pg/ml/month | −6.734 | 1.24 | 0.0012 | <0.0001 | 0.965 | 0.900-0.992 |

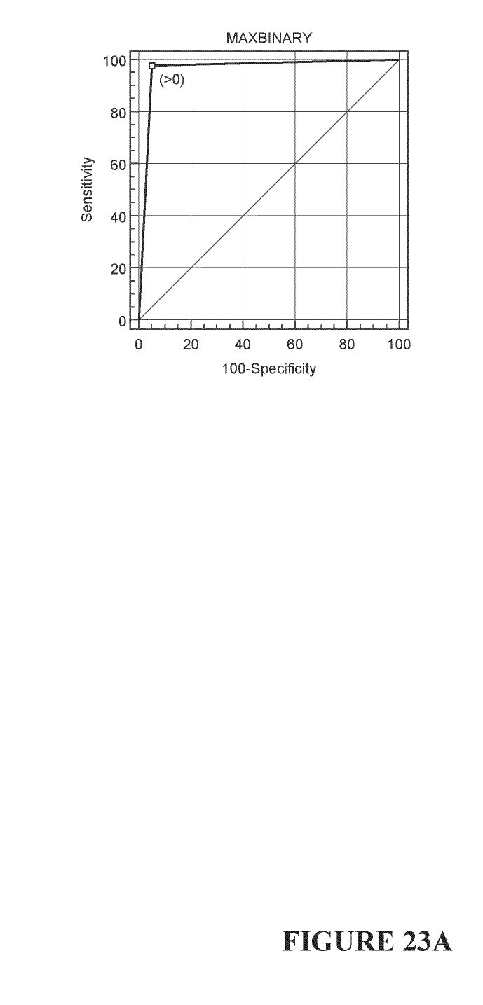

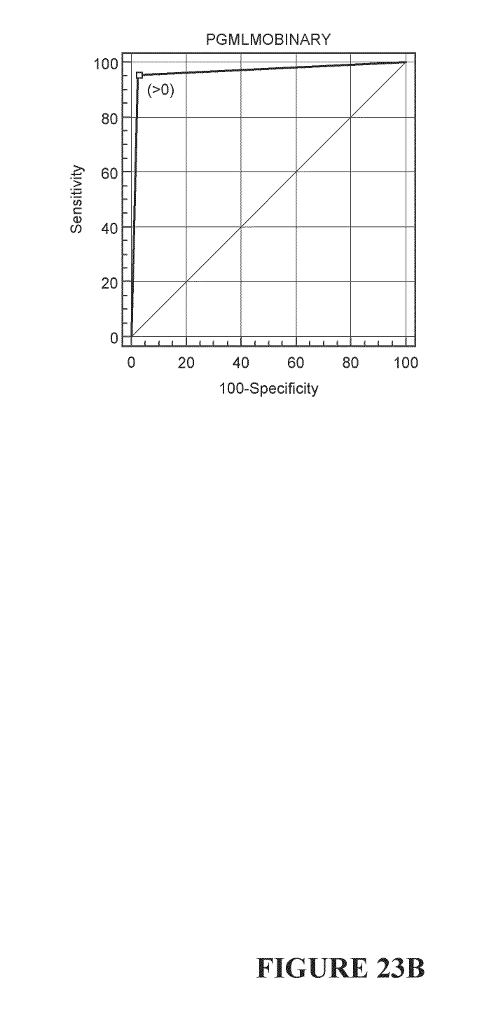

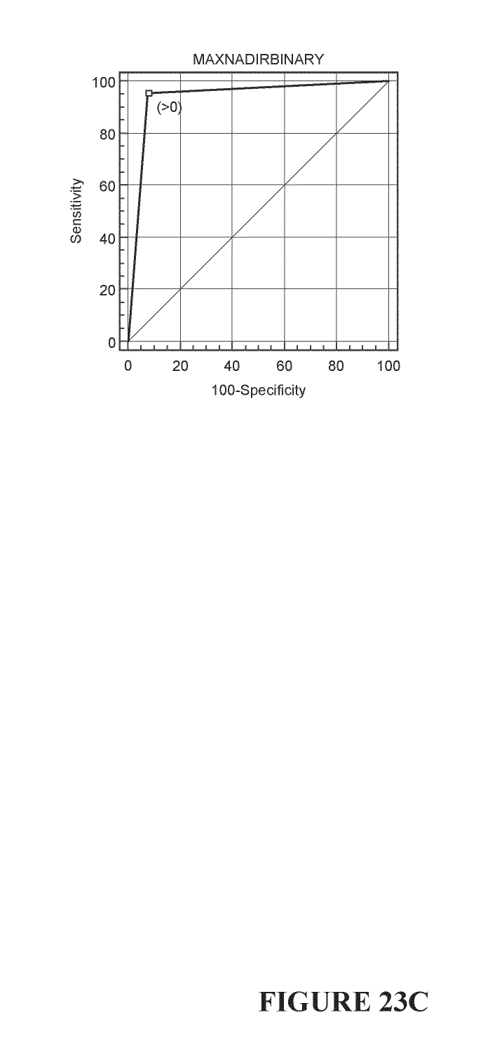

As shown in FIGS. 23A-C, the univariate analysis for each [PSA] index showed that the binary representations of these [PSA] indices were all very powerful, with p-values <0.0001 and AUC values approaching 1.0.

Conclusions on the Study:

The ® tPSA assay having a detection limit at least as low as 0.2 pg/mL and a functional sensitivity at least as low as 0.5 pg/mL can reliably measure tPSA concentration as low as 0.5 pg/mL providing precise PSA nadir results and PSA-doubling time calculations. Measurement of tPSA using a PSA assay having a low functional sensitivity at least as low as 0.5 pg/mL, such as ® assays, showed that the group of stable disease patients has a low and constant level of PSA with an approximate mean of 3.5 PG/ML (0.0035 ng/Ml). The difference between the patients having stable disease and the patients having biochemical recurrence is highly statistically significant.

In addition, on average, PSA-RPI assays detected a rising tPSA 34 months before the tPSA value reached 100 pg/mL (0.1 ng/mL).

The maximum observed PSA level is a very powerful index of stable disease or biochemical recurrence subpopulations. The maximum observed PSA level obtained using a PSA assay having a functional sensitivity at least as low as 0.5 pg/mL can be used to detect biochemical relapse early. The pg/mL/month increase is also a very powerful index of subpopulations having stable disease or biochemical recurrence subpopulations. The ratio of maximum observed PSA level to the nadir level is also a very powerful index of subpopulations having stable disease or biochemical recurrence.

The ® assay study showed that the tPSA parameters which served as the most discriminating indices of sub-populations (stable disease and early stage biochemical recurrence) were the maximum observed level, 2nd consecutive pg/mL/month increase rate, and doubling time.

### Example 8

### Calculations of Significance of Differences for Patients for Whom Earlier Data Was not Available

Calculation of the number of days required to reach [PSA] of 10, 25, 100, and 200 pg/mL when tPSA was measured using ® assays on the sample set was performed based on exponential fitting of 40 recurring and 10 stable disease patients. This type of analysis enables a comparison of recurring and stable disease populations at very early time points following radical prostatectomy. In this retrospective study, extrapolation based on the available data fit exponentially led to greater percentage error in the determination of small values associated with the time required for recurring patients to reach 10 pg/mL PSA. However, the results for the time required to reach 25, 100, and 200 pg/ml displayed increased confidence. Wilcoxon rank sum analysis was used to determine the significance of the differences between the two sub-populations. As shown in Table 24, below, the specified levels of [PSA] were reached significantly earlier in the recurring disease population than the stable disease population.

TABLE 24
Days required to reach various pg/ml PSA levels based on exponenetial fitting

| | Days to reach 10 | | Days to reach 25 | | Days to reach | | Days to reach | |
|---|---|---|---|---|---|---|---|---|
| | pg/ml, Mean | | pg/ml, Mean | | 100 pg/ml, Mean | | 200 pg/ml, Mean | |
| Population | (SD) | | (SD) | | (SD) | | (SD) | |
| Total (N = 50) | 491.6 | (1396.2) | 1147.8 | (1834.7) | 2140.7 | (2600.0) | 2637.2 | (3003.6) |
| Recurring (N = 40) | −26.7 | (762.5) | 394.4 | (745.0) | 1031.5 | (833.7) | 1350.0 | (920.1) |
| Stable (N = 10) | 2564.7 | (1457.7) | 4161.6 | (1818.3) | 6577.7 | (2539.6) | 7785.7 | (2938.3) |

| | | | | |
|---|---|---|---|---|
| p, Recurring v. Stable* | <0.0001 | <0.0001 | <0.0001 | <0.0001 |

*Wilcoxon rank sum

Calculation of ® assay pg/mL PSA at various time points (3, 6, 9, 12, and 18 months) were based on exponential fitting of 40 recurring and 10 stable disease patients. Wilcoxon rank sum analysis was used to determine the significance of the differences between the two sub-populations. As shown in Table 25, below, all of the values at a given point in time are higher in the recurring subpopulation than in the stable disease subpopulation. The significance of the difference increases with time, finally reaching p<0.001 at 18 months. This indicates that the populations consistently diverge with time.

TABLE 25
PSA pg/ml at various time points calculated by exponential fitting

| | pg/ml at 3 | pg/ml at 6 | pg/ml at 9 | pg/ml at 12 | pg/ml at 18 |
|---|---|---|---|---|---|
| | months, Mean | months, Mean | months, Mean | months, Mean | months, Mean |
| Population | (SD) | (SD) | (SD) | (SD) | (SD) |
| Total (N = 50) | 40.7 (153.1) | 53.3 (208.3) | 71.0 (284.0) | 97.1 (387.6) | 228.8 (833.9) |
| Recurring (N = 40) | 50.2 (170.2) | 65.9 (231.8) | 87.9 (316.0) | 120.4 (431.2) | 285.0 (926.1) |
| Stable (N = 10) | 3.0 (1.8) | 3.2 (2.0) | 3.4 (2.1) | 3.6 (2.3) | 4.1 (2.8) |
| p, Recurring v. Stable* | 0.0035 | 0.0012 | 0.0006 | 0.0002 | <0.0001 |

*Wilcoxon rank sum

### Example 9

### Use of Velocity as an Index of EC-BCR

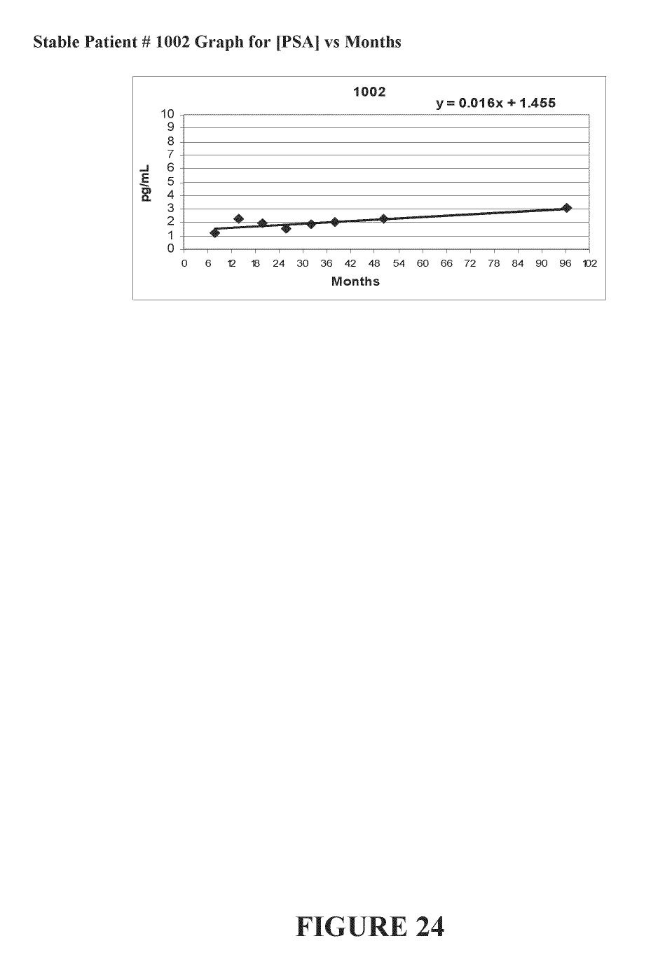

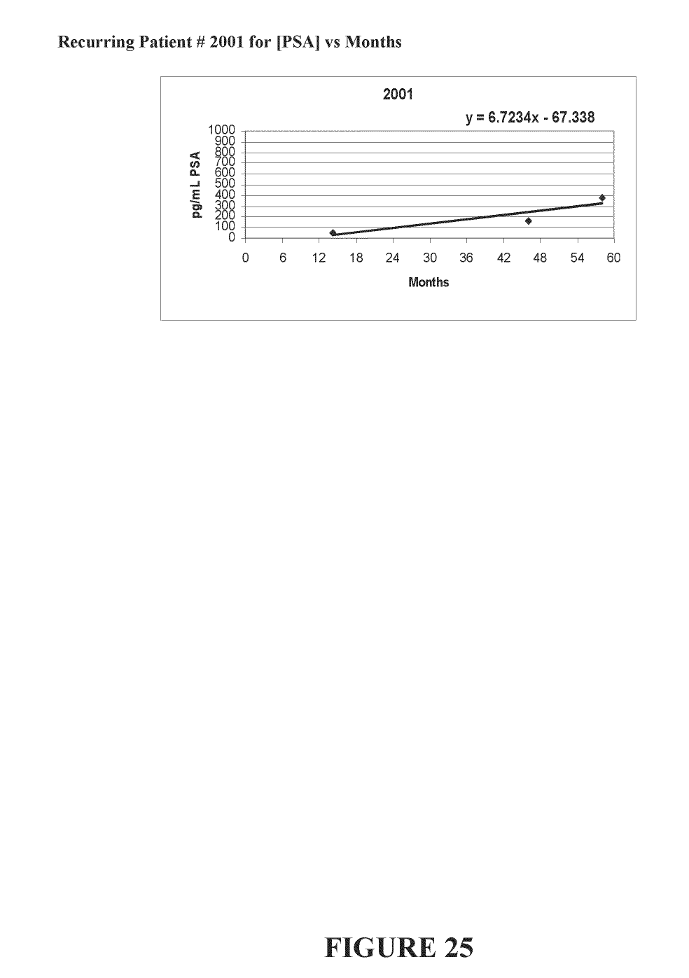

A retrospective study was completed comparing the linear plot of [PSA] post radical prostatectomy vs time for 16 stable patients and 13 recurring patients, over a period up to eight years. This study used the assay to measure total [PSA] as described in examples 1-6. The stable patients were defined as stable if the patient had no indication of recurrence of prostate cancer during the study period. A patient was defined as recurring if they had either a positive bone scan for prostate cancer recurrence and or death due to prostate cancer. The level of [PSA] was determined using the assay over a time period of approximately eight years. A linear curve fit was calculated for each patient. An example of the linear curve fit is shown for a stable patient (#1002) (FIG. 24) and for a recurring patient (#2001) (FIG. 25).

The slopes for each of the patients were determined and listed in Table 26 below:

TABLE 26
Slope of the linear curve for each of the stable and recurring patients
is included below:

| | | Slope of Linear | | Slope of |
|---|---|---|---|---|
| | | Curve (PSA | | Linear Curve |
| | | pg/ml per | | (PSA pg/ml |
| | Stable Patient # | Month) | Recurrent Patient # | per Month) |
| 1 | 1001 | 0.001 | 2001 | 6.723 |
| 2 | 1002 | 0.016 | 2002 | 26.604 |
| 3 | 1003 | 0.024 | 2003 | 13.035 |
| 4 | 1004 | 0.012 | 2004 | 16.290 |
| 5 | 1005 | −0.086 | 2005 | 29.044 |
| 6 | 1006 | 0.106 | 2006 | 22.712 |
| 7 | 1007 | 0.003 | 2007 | 30.255 |
| 8 | 1008 | 0.049 | 2008 | 10.004 |
| 9 | 1009 | 0.020 | 2009 | 70.460 |
| 10 | 10010 | 0.472 | 20010 | 39.419 |
| 11 | 10011 | 0.022 | 20012 | 41.681 |
| 12 | 10012 | 0.005 | 20013 | 6.576 |
| 13 | 10013 | −0.075 | 20014 | 7.523 |
| 14 | 10014 | −0.006 | | |
| 15 | 10015 | 0.0001 | | |
| 16 | 10016 | 0.041 | | |
| | Max. Value | 0.472 | | 70.46 |
| | Min. Value | −0.086 | | 6.58 |
| | Average Value | 0.038 | | 24.641 |

The maximum slope value for the stable patient group (pg/ml-month) is 0.472, or over 13 times lower than the minimum slope value of 6.58 from the group of recurring patients. The data demonstrated that using this high sensitivity assay provides a 100% discrimination between stable and recurring patients for prostate cancer, if one assumes a patient is not having a recurrence of prostate cancer post radical prostatectomy if the slope of the [PSA] vs time is less than 1. Note there was also a significant difference between the average values for each group of patients.

### Example 10

### Administration of Post-Prostatectomy Therapy Based on Determination of Fast, Medium or Slow ES-BCR

[PSA] values are obtained for post-prostatectomy patients, as described above. A PSA rate value, such as doubling time is determined, in order to discriminate between further subclasses of the recurring subpopulation of patients.

The analysis of PSA doubling time permits further sorting of patients into three groups, characterized by (1) a doubling time equal to or less than about ten months, which indicates fast or rapid recurrence; (2) a doubling time of more than about ten months up to equal to or about 24 months, which indicates medium ES-BCR, and (3) characterized by a doubling time of more than about 24 months, which indicates slow ES-BCR.

Patients displaying fast recurrence are administered post-prostatectomy therapy using external radiation therapy.

Clinical observations or pathological findings of Gleason scores, and wound margins are obtained for patients displaying medium or slow recurrence. Patients younger than 60 years old with Gleason scores >7 and poor margins who display medium or slow recurrence are administered post-prostatectomy therapy using external radiation therapy.

Patients older than eighty years old who display slow recurrence do not receive additional therapy.

Other patients are monitored for biochemical recurrence.

### Example 11

### Odds of Clinical Recurrence by Pathological Risk Factors and PSA-RPI Slope

Prostate cancer is a complex disease, and it has proved difficult to accurately predict clinical outcomes in individual men following RP. (Shariat S F, Kattan M W, Vickers A J, et al: Critical review of prostate cancer predictive tools. Future Oncology 2009; 5: 1555-1584.) Previous studies have linked undetectable PSA levels <0.01 ng/mL (<10 pg/mL) post-RP to relapse-free survival and nadir values to a lower likelihood for BCR. (Shen S, Lepor H, Yaffee R, et al: Ultrasensitive serum prostate specific antigen nadir accurately predicts the risk of early relapse after radical prostatectomy. J Urol 2005; 173: 777-80). However, the risk of BCR using assays with limits of quantitation higher than 2 pg/mL (such as 10 pg/mL or 5 pg/mL), is not informative as a predictor of clinical endpoints due to the apparently unpredictable kinetics of post-RP PSA values after a BCR event occurs—the result of trying to measure [PSA] levels below the limit of quantitation of those assays. (Moreira, D. M., Presti, J. C., Aronson, W. J., et al: Natural history of persistently elevated prostate specific antigen after radical prostatectomy: Results from the SEARCH database. J. Urol. 2009; 182: 2250-2256. D'Amico, A. V., Moul, J. W., Carroll, P. R., et al: Surrogate marker for prostate cancer-specific mortality following radical prostatectomy or radiation therapy. J. Natl. Cancer Inst. 2003; 95: 1376-1383. Amling, C. L., Bergstralh, E. J., Blute, M. L., et al: Defining prostate specific antigen progression after radical prostatectomy: what is the most appropriate cut point? J. Urol. 2001: 165: 1146-1151. Stephenson, A. J., Kattan, M. W., Eastham, J. A., et al. Defining biochemical recurrence of prostate cancer after radical prostatectomy: a proposal for a standardized definition. J. Clin. Oncol. 2006; 24: 3973-3978. Shinghal, R., Yemotom, C., McNeal, J. E., et al: Biochemical recurrence without PSA progression characterizes a subset of patients after radical prostatectomy. Urology 2003; 61: 380-385. For this reason, the pre-IDE protocol stipulated that the endpoint must be clinically documented recurrence rather than BCR for determining the efficacy of PSA-RPI. Patients with a [PSA] slope value ≦a PSA-RPI of 2.0 pg/mL/month enjoyed a 90% reduced risk for clinical recurrence in multivariate analyses. In a multivariate model that included pre-RP [PSA], pathologic stage and Gleason score, PSA-RPI slope was the most powerful independent prognostic factor for identifying men at reduced risk for clinical recurrence (HR 9.8, P<0.0001). While Gleason score (HR 5.4) was also a predictor, PSA-RPI slope was the strongest independent prognostic index of recurrence risk, including the subset of men having pathologic Gleason score ≧7 cancers.

A recently developed and validated immuno-polymerase chain reaction (IPCR) assay was used for measuring total serum PSA in human samples. (McDermed J E, Sanders R, Fait S, et al: Nucleic acid detection immunoassay for prostate specific antigen based on immuno-PCR methodology. Clin. Chem. 2012; 58: 732-740.) The dual-antibody sandwich IPCR assay uses in some aspects a double-stranded (ds) DNA label for analyte detection, but also incorporates features that resist non-specific binding and improve analytical sensitivity and precision. The limit of quantification (LOQ) of this validated assay is 0.00065 ng/mL (0.65 pg/mL), roughly an order of magnitude lower than the most sensitive commercial PSA assays. Further, in some aspects the assay employs an index based on the change in [PSA] over time post-RP as a unique prognostic factor. In pilot studies measuring [PSA] using a PSA assay with an LOQ lower than 2 pg/mL, it was determined that most men have serum PSA concentrations well below 10 pg/mL in the first several months following RP. (Moul, J. W., Semmes, O. J., Vessella, R. L., et al: Prognostic impact of post-prostatectomy PSA slope determined with a novel, nucleic acid detection immunoassay ProsVue for total PSA. J. Clin. Oncol. 2011; 29 (Suppl 7): 47.)

In addition, in some aspects the superior sensitivity of the assays of this invention gave precise [PSA] determinations to below 1 pg/mL and provided a unique opportunity to evaluate the kinetics of changes in [PSA] at these very low post-RP levels. It was further determined that [PSA] change over time exhibited first-order kinetics in non-recurring men and that the rate of change (in pg/mL/month) was significantly different from men who suffered from clinical recurrence. (Moul J, Vessella R, Semmes O J, et al: PSA slope identifies men at reduced risk for prostate cancer recurrence post-radical prostatectomy: A multicenter study of the ® ProsVue™ assay. 2011 AUA Annual Meeting Proceedings; abstr. 1915.)

These studies determined the potential clinical utility of a [PSA] slope cutpoint, or [PSA] slope recurrence probability index (PSA-RPI) of 2.0 pg/mL/month and the hypothesis that men displaying a slope ≦2.0 pg/mL/month post-RP could be at a reduced risk of clinical recurrence.

The study protocol was developed in conjunction with the U.S. Food and Drug Administration and cleared through the pre-Investigational Device Exemption (IDE) process. Its objective was to validate the hypothesis that men displaying PSA slopes ≦2.0 pg/mL/month post-RP were at a reduced risk of clinical recurrence in an appropriately powered case-cohort designed trial. Prentice RL: A case-cohort design for epidemiologic cohort studies and disease prevention trials. Biometrika 1986; 73: 1-11.

These results provided the basis for United States 510(k) clearance of ProsVue™ as a post-RP prognostic marker. From 4 clinical sites, we selected a cohort of 304 men followed up to 17.6 years postprostatectomy for clinical recurrence. We assessed the prognostic value of a PSA slope cutpoint of 2.0 pg/mL/month against established risk factors to identify men at very low risk of clinical recurrence using uni- and multivariate Cox proportional hazards regression and Kaplan-Meier analysis. The univariate HR (95% CI) of a PSA slope >2.0 pg/mL/month was 18.3 (10.6-31.8), compared to a slope ≦2.0 pg/mL/month (P<0.0001). Median disease-free survival was 4.8 years versus >10 years in the 2 groups (P<0.0001). Multivariate HR for PSA slope with the covariates of preprostatectomy PSA, pathologic stage and Gleason score was 9.8 (5.4-17.8), an 89.8% risk reduction, for men with PSA slopes ≦2.0 pg/mL/month (P<0.0001). Gleason Score (<7 vs. ≧7) was the only other significant predictor (HR 5.4, 2.1-13.8, P=0.0004). Although clinical recurrence following radical prostatectomy is often difficult to predict since established factors do not reliably stratify risk, results show that men displaying serum PSA slopes <2.0 pg/mL/month postprostatectomy, measured with a new immuno-PCR diagnostic test (ProsVue™) were at a reduced risk of clinical recurrence as determined by positive biopsy, imaging or death due to prostate cancer, and that the PSA time value such as PSA slope added predictive power to established risk factors.

### Example 11A

### Patients and Methods

Study Design

The required sample size assuming a type I error rate of 5%, 80% power, and a univariate hazard ratio (HR) for PSA slope of 1.4 (estimated from pilot studies) based on a two-sided test was 262. Assuming 20% prevalence for recurrence, a minimum of 52 men with recurrence was required. A cohort random sampling of eligible men was performed within four strata arising from categorization as above or below median patient age (61.4 years) and median sample storage time (13.5 years). The final selected study population consisted of 64 men with clinical recurrence and 240 controls (prevalence 21.1%). The linear slope of PSA in pg/mL vs. time post-RP in months (PSA-RPI) was calculated for each man via least squares linear regression. PSA-RPI slope was expressed as a binary categorical variable with the cutpoint set at 2.0 pg/mL/month for clinical investigation. A slope of ≦2.0 pg/mL/month was the pre-specified hypothesis for reduced risk of recurrence, therefore a slope >2.0 pg/mL/month denoted men “not at reduced risk for recurrence.”

Study Population

The study was prospectively designed and incorporated archived serum samples from men treated by RP at four investigational sites (Duke University, Eastern Virginia Medical School, Memorial Sloan-Kettering Cancer Center, and the University of Washington) during 1990-2001. Institutional review boards at all sites approved the study protocol. Samples for analysis were de-identified such that no personally identifiable health information was released by the sites except for dates of birth, death, and clinical procedures, which were used to calculate age at RP, time to recurrence or length of follow-up. Inclusion criteria consisted of men with biopsy-confirmed prostate cancer treated by RP with a PSA value in the first post-RP sample <0.1 ng/mL (<100 pg/mL) using standard-of-care immunoassays for PSA. Three serum samples obtained between 1.5 and 20 months post-RP were required with at least a two-month interval between each sampling time. Documentation of pre-RP PSA, pathologic stage and Gleason score was required to serve as covariates in multivariate analyses. Findings of positive surgical margins, extracapsular extension, seminal vesicle invasion, bladder neck invasion and positive lymph nodes were also recorded if documentation was available. Men categorized by the sites as non-recurring required a minimum post-RP follow-up of 8 years. Exclusion criteria were radiation treatment administered in the first 12 months post-RP, androgen deprivation therapy administered in the first 20 months post-RP, and serum samples stored >20 years.

Criteria for Response

Men categorized by the sites as recurrent required documented evidence of local recurrence by biopsy, evidence of metastases by imaging methods (MRI, CT, bone scan, or ¹¹¹In immunoscintigraphy in conjunction with CT), or death due to prostate cancer. Patients categorized as non-recurrent (no clinical evidence of disease) were followed after RP a median of 11.0 (IQR, 9.6-12.9) years versus a median of 4.7 (IQR, 2.7-8.4 years) for patients with clinically recurrent disease. Men with BCR without documented disease were categorized as non-recurrent.

Clinical Samples

Serum samples from 433 men treated by RP were available for this study. Samples were stored at ≦−70° C. in each site's biorepository. The dates samples were drawn and the PSA immunoassay method used were documented in medical records. Samples from 24 men could not be used because the men either did not meet protocol eligibility or were excluded because secondary treatment was administered during the sample collection period. A total of 1,227 archived samples from the 409 men eligible for cohort selection were shipped on dry ice.

PSA Assay

ProsVue is a re-engineered IPCR assay that utilizes a non-native dsDNA label for analyte detection (IRIS International, Inc.). Details of the PSA assay procedure have been previously described. (McDermed J E, Sanders R, Fait S, et al: Nucleic acid detection immunoassay for prostate specific antigen based on immuno-PCR methodology. Clin Chem 2012; 58: 732-740.) Briefly, PSA is first reacted in solution with a monoclonal antibody (MAb) directed to one epitope on the PSA molecule that is conjugated to a 59-base non-native double stranded (ds) DNA label. Following 2-hour incubation at ambient temperature, a second MAb targeting a second epitope of the PSA molecule and immobilized on paramagnetic microparticles is added and incubated for another 30 minutes. After the particles are washed, the dsDNA label is amplified by PCR and the quantification cycle (Cq) is measured automatically on an Applied Biosystems 7500 FastDx real time PCR instrument (Life Technologies, Inc.). Cq is plotted vs. log₁₀ PSA concentration with linear regression analysis. Each sample is assayed in duplicate and re-assayed in duplicate if the coefficient of variation (CV) is >20%. The assay's linear range extends beyond its reportable range (0.65-100 pg/mL), and intra-assay precision is <9.0%. (McDermed, J. E., Sanders, R., Fait, S., et al: Nucleic acid detection immunoassay for prostate specific antigen based on immuno-PCR methodology. Clin. Chem. 2012; 58: 732-740.)

Statistical Analyses

Demographic variables and available clinicopathological covariates were analyzed descriptively. Continuous variables were compared via the Wilcoxon rank sum test. Categorical variables were compared via the Pearson chi-square or Fisher's exact tests, as appropriate. Univariate and multivariate Cox proportional hazards regression models were performed to determine the predictive capability for risk of recurrence of the binary expression of the PSA slope and covariates. Time to recurrence was calculated via subtracting the RP date from the date of the first documented event, either local recurrence, distant metastases or death due to prostate cancer. Length of follow-up for non-recurrent patients was determined by subtracting the RP date from the last date of patient follow-up. If a death not related to prostate cancer occurred in this group, the date of death was used to calculate length of follow-up. All follow-up times were expressed in years. A Kaplan-Meier plot of the univariate survival probabilities (proportion of men without clinical recurrence or death due to prostate cancer vs. length of follow-up) of men categorized by the PSA-RPI slope cutpoint as at reduced risk vs. those not at reduced risk was constructed. In addition, Kaplan-Meier plots were constructed for men according to the final pathologic findings (positive and negative) and differences in survival were compared via the Wilcoxon and log-rank tests. Sensitivity, specificity and positive and negative predictive values were calculated via a standard 2×2 table of clinical recurrence categorization vs. PSA-RPI slope binary categorization and exact binomial 95% confidence intervals (CI) were determined. SAS V9.1 (SAS Institute, Cary, N.C.), JMP v5.01 (SAS Institute), and NCSS 2007 (NCSS Inc., Kaysville, Utah) were used for the statistical analyses. All tests were two-sided and P<0.05 was considered significant.

### Example 11B

### Results

Study Population

Table 11A displays the distributions of demographic and clinicopathological variables for the studied population which included Caucasian (88.8%), African-American (8.2%), and Asian (2.0%) men with a median age of 61.4 (IQR, 59.6-66.1) years at RP. Tumors ranged from pathologic Gleason score four to nine, were primarily pT2, and were often associated with positive surgical margins and extracapsular extension. Comparing the recurrent and non-recurrent groups, pre-RP PSA (mean 10.53 vs. 6.88 ng/mL), tumor volume (mean 26.5 vs. 15.7%), pathologic stage T3/T4 (78.1 vs. 34.6%), Gleason score ≧7 (59.2 vs. 43.7%), positive surgical margins (42.2 vs. 24.4%), extracapsular extension (75.0 vs. 32.1%), seminal vesicle invasion (37.5 vs. 5.4%) and bladder neck invasion (10.9 vs. 4.6%) were higher in the recurrent vs. non-recurrent groups, respectively, indicating increased aggressiveness of cancers in the men with documented clinical recurrence.

TABLE 11A
Clinicopathologic characteristics of the studied population

| Characteristic | N | Pct. | Mean | Median (IQR) |
|---|---|---|---|---|
| Age at RP (years) | 304 | — | 61.3 | 61.4 (59.6-66.1) |
| Percent tumor volume | 65 | — | 18.3 | 15.0 (6.5-25.0) |
| Pre-RP treatment | 59 | 19.4 | | |
| Pre-RP PSA (ng/mL) | | | | |
| ≦4.0 | 70 | 23.0 | | |
| 4.0-10.0 | 163 | 53.6 | | |
| 10.1-20.0 | 57 | 18.8 | | |
| >20.0 | 14 | 4.6 | | |
| Pathologic GS | | | | |
| <6 | 44 | 14.5 | | |
| 6 | 96 | 31.6 | | |
| 7 | 126 | 41.4 | | |
| >7 | 38 | 12.5 | | |
| Pathologic stage | | | | |
| T0 | 2 | 0.7 | | |
| T2 | 168 | 55.4 | | |
| T3 | 120 | 39.4 | | |
| T4 | 14 | 4.6 | | |
| Other pathologic findings¹ | | | | |
| Surgical margin positive | 95 | 31.3 | | |
| Extracapsular extension | 125 | 41.4 | | |
| Seminal vesicle involvement | 37 | 12.4 | | |
| Bladder neck invasion | 18 | 11.0 | | |
| Lymph node involvement | 8 | 3.0 | | |
| Race¹ | | | | |
| African-American | 25 | 8.3 | | |
| Asian | 6 | 2.0 | | |
| Caucasian | 270 | 89.7 | | |
| Ethnicity¹ | | | | |
| Hispanic | 8 | 4.4 | | |
| Non-Hispanic | 173 | 95.6 | | |

PSA Results

Median sampling times across the study cohort were 4.9, 8.5, and 12.8 months after RP. In the non-recurring group, the median PSA value in the first post-RP serum sample was 3.1 interquartile range (IQR) of 1.8-6.6 pg/mL. Median PSA values in the second and third samples showed minimal change and were 3.4 (IQR, 2.0-7.1) pg/mL and 4.1 (IQR, 2.2-8.4) pg/mL respectively. In the clinically recurring group, median PSA values showed a significant rise across the three sampling points. Median PSA values of the first, second and third samples were 14.1 (IQR, 4.1-48.4) pg/mL, 45.0 (IQR, 10.2-104.4) pg/mL and 78.4 (IQR, 17.7-211.0) pg/mL, respectively. The median PSA-RPI slope calculated from the three samples was 0.03 (IQR, −0.04-0.24) pg/mL/month in the non-recurrent group compared to 5.6 (1.6-22.0) pg/mL/month in the clinically recurring group (P<0.0001).

Efficacy Analysis

Clinical recurrence was documented in 64/304 (21.1%) men; 15 initially recurred with biopsy-proven localized disease and 49 had distant metastases as their first clinically recurrent event. Skeletal metastases initially presented in 24/64 (37.5%) Of the 64 men that recurred, 46/64 (71.9%) were correctly classified as “not at reduced risk” of recurrence based on a PSA-RPI slope >2.0 pg/mL/month. Recurrence did not occur in 240 men of whom 227/240 (94.5%) were correctly classified as “at reduced risk” of recurrence based on a PSA-RPI slope ≦2.0 pg/mL/month. The study hypothesis was to validate PSA-RPI for identifying men at a reduced risk for recurrence, sensitivity, specificity, positive predictive value (PPV) and negative predictive value (NPV) for clinical recurrence were determined. Sensitivity (95% CI) was 71.9% (59.2-82.4%) at 94.6% (90.9-97.1%) specificity. PPV and NPV at the 21.1% prevalence of recurrence in the present study were 78.0% (65.3-87.7%) and 92.7% (88.6-96.5%), respectively.

PSA-RPI Slope and Pathological Endpoints

Table 11B summarizes the risk of clinical recurrence according to the post-RP PSA slope and pathologic variables using uni- and multivariate Cox proportionate hazards regression analysis. A PSA-RPI slope ≦2.0 pg/mL/month post-RP was significantly associated with a reduced risk of clinically recurrent prostate cancer by univariate Cox proportional hazards regression analysis (HR 18.3, 95% CI, 10.6-31.8, P<0.0001). Most covariates and other pathologic variables were also significantly associated with clinical recurrence using univariate analysis. In the multivariate model including pre-RP PSA, pathologic stage, and Gleason score as covariates, the HR for PSA-RPI slope was minimally attenuated to 9.8 (95% CI, 5.4-17.8, P <0.0001) and was an independent predictor of recurrence risk. Taking the inverse of the HR yields a ratio of 0.102 or an 89.8% risk reduction for men with a PSA-RPI slope ≦2.0 pg/mL/month. Of the covariates, only Gleason score was significantly associated with recurrence risk (HR 5.4, 95% CI, 2.1-13.8, P=0.0004) or an 81.4% reduction in risk for men with Gleason score <7.

TABLE 11B
Univariable and multivariate Cox regression analysis of the risk of clinical
recurrence according to the post-RP PSA-RPI slope and pathologic variables

| Clinicopathologic | | Median | No. | Univariate Analysis | | Multivariate Analysis | |
|---|---|---|---|---|---|---|---|
| variables | N | Follow-up | Events | HR (95% CI) | P | HR (95% CI) | P |
| PSA slope | | | | | | | |
| ≦2.0 | 245 | 10.6 | 18 | 1.0 | | 1.0 | |
| >2.0 | 59 | 7.8 | 46 | 18.3 (10.6- | <0.0001 | 9.8 (5.4- | <0.0001 |
| Pre-RP PSA level | | | | | | | |
| (ng/mL) | | | | | | | |
| Continuous | 304 | 8.6 | 64 | 1.1 (1.03- | <0.0001 | 1.0 (0.98- | 0.647 |
| Pathologic Gleason | | | | | | | |
| 4, 5, 6 | 140 | 10.9 | 5 | 1.0 | | 1.0 | |
| 7, 8, 9 | 164 | 10.0 | 59 | 12.1 (4.8- | <0.0001 | 5.4 (2.1- | 0.0004 |
| Pathologic stage | | | | | | | |
| pT0-pT2 | 170 | 10.8 | 13 | 1.0 | | 1.0 | |
| pT3-pT4 | 134 | 9.7 | 51 | 5.8 (3.2- | <0.0001 | 1.7 (0.9-3.4) | 0.105 |
| Other pathologic findings¹ | | | | | | | |
| Percent tumor volume | | | | | | | |
| Continuous | 65 | 10.1 | 16 | 1.0 (1.0- | 0.059 | | ND² |
| Surgical margins | | | | | | | |
| Negative | 209 | 10.5 | 27 | 1.0 | | | |
| Positive | 95 | 9.9 | 37 | 3.3 (2.0- | <0.0001 | | ND |
| Extracapsular | | | | | | | |
| Absent | 177 | 10.8 | 15 | 1.0 | | | |
| Present | 125 | 9.7 | 48 | 5.3 (3.0- | <0.0001 | | ND |
| Seminal vesicle | | | | | | | |
| Absent | 261 | 10.5 | 40 | 1.0 | | | |
| Present | 37 | 8.8 | 24 | 5.3 (3.2- | <0.0001 | | ND |
| Bladder neck | | | | | | | |
| Absent | 146 | 10.5 | 24 | 1.0 | | | |
| Present | 18 | 9.8 | 7 | 2.8 (1.2- | 0.016 | | ND |

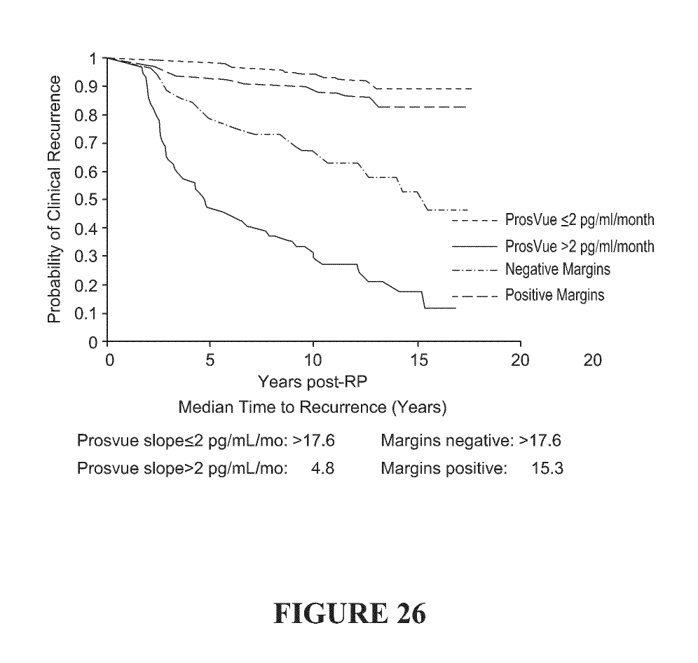

FIG. 26 displays the Kaplan-Meier plot of the univariate survival probabilities for men at reduced risk and not at reduced risk for recurrence based on the PSA slope cutpoint of 2.0 pg/mL/month. The survival curves diverged within the first two years of follow-up (Wilcoxon P <0.0001) and continue to separate due to the minimal decline in surviving proportion of the men with a PSA slope ≦2.0 pg/mL/month. Median survival was 4.8 years and >10 years in the groups of men with a PSA slope >2.0 and ≦2.0 pg/mL/month, respectively (log-rank P<0.0001).

In the Gleason score ≧7 subset, sensitivity was 71.2% (57.9-82.2%) at 91.4% (84.4-96.0%) specificity and PPV and NPV were 82.4% (69.1-91.6%) and 85.0% (77.0-91.0%) respectively. Multivariate Cox proportional hazards regression analysis including pre-RP PSA and pathologic stage (T3-T4 vs. T0-T2) as covariates showed minimal attenuation of the HR for PSA-RPI slope to 8.1 (95% CI, 4.5-14.7, P<0.0001) and it remained the strongest independent predictor of recurrence risk.

### Example 11C

Prostate cancer is a complex disease, and has proved difficult to predict clinical outcomes accurately in individual men following RP. Shariat S F, Kattan M W, Vickers A J, et al: Critical review of prostate cancer predictive tools. Future Oncology 2009; 5: 1555-1584. Previous studies have linked undetectable PSA levels <0.01 ng/mL (<10 pg/mL) post-RP to relapse-free survival (Doherty, A. P., Bower, J., Smith, G. L., et al: Undetectable ultrasensitive PSA after radical prostatectomy for prostate cancer predicts relapse-free survival. Br. J. Cancer 1999; 83: 1432-1436.) and nadir values to a lower likelihood for BCR. Shen, S., Lepor, H., Yaffee, R., et al: Ultrasensitive serum prostate specific antigen nadir accurately predicts the risk of early relapse after radical prostatectomy. J. Urol 2005; 173: 777-80.) However, the risk of BCR is not informative as a predictor of clinical endpoints because of the unpredictable kinetics of post-RP PSA values after a BCR event occurs. (Moreira, D. M., Presti, J. C., Aronson, W. J., et al: Natural history of persistently elevated prostate specific antigen after radical prostatectomy: Results from the SEARCH database. J. Urol 2009; 182: 2250-2256. D'Amico, A. V., Moul, J. W., Carroll, P. R., et al: Surrogate marker for prostate cancer-specific mortality following radical prostatectomy or radiation therapy. J. Natl. Cancer Inst. 2003; 95: 1376-1383. Amling, C. L., Bergstralh, E. J., Blute, M. L., et al: Defining prostate specific antigen progression after radical prostatectomy: what is the most appropriate cut point? J. Urol 2001: 165: 1146-1151. Stephenson, A. J., Kattan, M. W., Eastham, J. A., et al. Defining biochemical recurrence of prostate cancer after radical prostatectomy: a proposal for a standardized definition. J. Clin. Oncol. 2006; 24: 3973-3978. Shinghal, R., Yemoto, C., McNeal, J. E., et al: Biochemical recurrence without PSA progression characterizes a subset of patients after radical prostatectomy. Urology 2003; 61: 380-385.) For this reason, the pre-IDE protocol stipulated that the endpoint must be clinically documented recurrence rather than BCR for determining the efficacy of PSA-RPI. Patients with a PSA-RPI slope ≦2.0 pg/mL/month enjoyed a 90% reduced risk for clinical recurrence in multivariate analyses. In a multivariate model that included pre-RP PSA, pathologic stage and Gleason score, PSA-RPI slope was the most powerful independent prognostic factor for identifying men at reduced risk for clinical recurrence (HR 9.8, P<0.0001). While Gleason score (HR 5.4) was also a predictor, PSA-RPI slope was the strongest independent prognostic index of recurrence risk, including the subset of men having pathologic Gleason score >7 cancers.

### Example 11D

### Conclusions

This is the first study of a diagnostic for risk stratification of clinical prostate cancer recurrence with >10 years median follow-up and PSA-RP is the first assay to receive FDA clearance based on linear slope of tumor marker concentration over time. In this study, PSA-RPI slope was the most powerful index of reduced risk of clinical recurrence and added predictive value to established risk factors. PSA-RPI could possibly reduce healthcare costs by reducing the intensity of follow-up in men identified at a reduced risk for recurrence. Further studies will assess method performance and define the role of the assay in risk models and nomograms.

### Example 12

### Combined Risk Factors

It was further discovered that certain pathological findings had an unexpectedly high predictive value for prostate cancer recurrence. As an example, it was discovered that the combination of the PSA RPI and one or more pathological findings was surprisingly highly correlated with a higher risk of clinical recurrence, and/or higher likelihood of stable disease, and added predictive value to established risk factors. These factors can include, for example, seminal vesicle invasion, Gleason score, extracapsular invasion and surgical margins.

In one embodiment, a combination of PSA time value exceeding the PSA-RPI and positive pathological findings provided an unexpectedly high predictive value for likelihood of prostate cancer recurrence, expressed in term of odds ratios for various pathological finding parameters across a wide range of PSA time indicator values (such as PSA RPI) of 0.2 to 5 pg/mL/month. The combination of PSA RPI and pathological findings is a strong predictor of recurrence, as discussed further below.

TABLE 12A

| | Odds | Odds Ratio | | |
|---|---|---|---|---|
| | Ratio For | For Positive | Odds Ratio For | |
| | extracapsular | Surgical | Gleason Score 7 | Odds Ratio For |
| Slope | Extension | Margins | and Higher | Seminal Vesicle |
| >5.0 | 7.70 | 6.90 | 9.49 | 5.96 |
| >2.0 | 6.06 | 5.88 | 8.31 | 4.88 |
| >1.0 | 5.40 | 5.40 | 6.82 | 3.61 |
| >0.5 | 3.56 | 2.81 | 4.45 | 3.11 |
| >0.2 | 2.50 | 2.39 | 2.80 | 2.49 |

TABLE 12B

| | Odds Ratio For | | | |
|---|---|---|---|---|
| | N0 | Odds Ratio For | Odds Ratio For | |
| | extracapsular | NO Positive | Gleason Score | Odds Ratio For |
| Slope | Extension | Surgical | <7 | NO Seminal |
| >5.0 | 39.60 | 31.46 | 54.00 | 23.02 |
| >2.0 | 29.70 | 21.57 | 27.00 | 15.47 |
| >1.0 | 10.80 | 9.15 | 9.82 | 9.21 |
| >0.5 | 5.40 | 5.39 | 4.50 | 4.51 |
| >0.2 | 3.69 | 3.29 | 3.27 | 3.09 |

Seminal Vesicle Invasion

Seminal vesicle invasion (SVI) is a significant risk factor for clinical recurrence of prostate cancer post-prostatectomy. Determination of a PSA time value such as a PSA RPI adds significantly more information to a risk assessment based on seminal vesicle invasion. For example, Table 12 A shows that the odds of clinical recurrence in men with SVI and a PSA-time value >a PSA RPI of 2.0 pg/mL/month is 4.88 times higher compared to men with SVI. As another example, as shown in Table 12 B, the odds of clinical recurrence in men without SVI and a PSA-time value >a PSA RPI of 2.0 pg/mL/month are 15.47 times higher compared to men without SVI.

Extracapsular Extension

Determination of a PSA-RPI adds significantly more information to a risk assessment based on extracapsular extension. For example, Table 12 A shows that the odds of clinical recurrence in men with CE and a PSA-time value >a PSA RPI of 2.0 pg/mL/month is 6.06 times higher compared to men with CE. As another example, as shown in Table 12 B, the odds of clinical recurrence in men without CE and a PSA-time value >a PSA RPI of 2.0 pg/mL/month are 29.70 times higher compared to men without CE.

Surgical Margins

Determination of a PSA RPI adds significantly more information to a risk assessment based on positive surgical margins (SM). For example, Table 12 A shows that the odds of clinical recurrence in men with SM and a PSA-time value >a PSA RPI of 2.0 pg/mL/month is 5.88 times higher compared to men with SM. As another example, as shown in Table 12 B, the odds of clinical recurrence in men without SM and a PSA-time value >a PSA RPI of 2.0 pg/mL/month are 21.57 times higher compared to men without SM.

Gleason Score

A pathological Gleason score (pGS) ≧7 is a significant risk factor for clinical recurrence of prostate cancer post-prostatectomy. As shown in Table 12A, the odds of clinical recurrence in men with a pGS ≧7 and a PSA-RPI >2.0 pg/mL/month are 8.31 times higher compared to men with a pGS ≧7. Determination of PSA-RPI adds significantly more information to this risk assessment. As shown in Table 12 B, the odds of clinical recurrence in men with a pGS <7 and a PSA-RPI >2.0 pg/mL/month is 27.0 times higher compared to men with a pGS <7.

In another embodiment, the combination of PSA time value not exceeding the PSA-RPI and negative pathological finding provided an unexpectedly high predictive value for likelihood of stable disease/or recurrence expressed in term of odds ratios for various pathological finding parameters across a wide range of PSA RPI values (0.2 to 5). The combination of PSA RPI and pathological findings is a strong predictor of stable disease.

TABLE 12C

| | Odds | Odds Ratio | | |
|---|---|---|---|---|
| | Ratio For | For Positive | Odds Ratio For | Odds Ratio For |
| | extracapsular | Surgical | Gleason Score 7 | Seminal Vesicle |
| Slope | Extension | Margins | and Higher | Invasion |
| ≦0.2 | 0.19 | 0.09 | 0.17 | 0.07 |
| ≦0.5 | 0.22 | 0.12 | 0.19 | 0.06 |
| ≦1.0 | 0.27 | 0.19 | 0.25 | 0.22 |
| ≦2.0 | 0.33 | 0.22 | 0.32 | 0.30 |
| ≦5.0 | 0.53 | 0.44 | 0.49 | 0.59 |

TABLE 12D

| | Odds Ratio For | | | |
|---|---|---|---|---|
| | N0 | Odds Ratio For | Odds Ratio For | |
| | extracapsular | NO Positive | Gleason Score | Odds Ratio For |
| Slope | Extension | Surgical | <7 | NO Seminal |
| ≦0.2 | 0.17 | 0.29 | 0.26 | 0.24 |
| ≦0.5 | 0.16 | 0.30 | 0.24 | 0.27 |
| ≦1.0 | 0.22 | 0.32 | 0.22 | 0.27 |
| ≦2.0 | 0.27 | 0.42 | 0.21 | 0.31 |
| ≦5.0 | 0.27 | 0.49 | 0.20 | 0.39 |

Seminal Vesicle Invasion

Seminal vesicle invasion (SVI) is a significant risk factor for clinical recurrence of prostate cancer post-prostatectomy. Determination of a PSA-time value such as PSA RPI adds significantly more information to assessment of likelihood of stable disease/or recurrence combined with observation of whether there is seminal vesicle invasion. For example, Table 12 C shows that the odds of clinical recurrence in men with SVI and a PSA-time value ≦a PSA RPI of 2.0 pg/mL/month are 0.30 times the odds of clinical recurrence in men with SVI. As another example, as shown in Table 12 D, the odds of clinical recurrence in men without SVI and a PSA-time value ≦a PSA RPI of 2.0 pg/mL/month are 0.31 times the odds of clinical recurrence in men with without SVI.

Extracapsular Extension

Determination of a PSA-RPI adds significantly more information to assessment of likelihood of stable disease/or recurrence combined with observation of whether there is extracapsular extension. For example, Table 12 C shows that the odds of clinical recurrence in men with CE and a PSA-time value ≦a PSA RPI of 2.0 pg/mL/month are 0.33 times the odds of clinical recurrence in men with CE. As another example, as shown in Table 12 D, the odds of clinical recurrence in men without CE and a PSA-time value ≦a PSA RPI of 2.0 pg/mL/month are 0.27 times the odds of clinical recurrence in men with without CE.

Surgical Margins

Determination of a PSA-RPI adds significantly more information to assessment of likelihood of stable disease combined with observation of whether there are positive surgical margins (SM). For example, Table 12 C shows that the odds of clinical recurrence in men with SM and a PSA-time value <a PSA RPI of 2.0 pg/mL/month are 0.22 times the odds of clinical recurrence in men with SM. As another example, as shown in Table 12 D, the odds of clinical recurrence in men without SM and a PSA-time value ≦a PSA RPI of 2.0 pg/mL/month are 0.42 times the odds of clinical recurrence in men without SM.

Gleason Score

A pathological Gleason score (pGS) ≧7 is a significant risk factor for clinical recurrence of prostate cancer post-prostatectomy. Determination of a PSA-RPI adds significantly more information to assessment of likelihood of stable disease/or recurrence combined with the Gleason score. The odds of clinical recurrence in men with a pGS ≧7 and a PSA-slope ≦2.0 pg/mL/month are 0.32 times the odds of clinical recurrence in men with a pGS ≧7. Determination of PSA-RPI adds significantly more information to this risk assessment. The odds of clinical recurrence in men with a pGS <7 and a PSA− slope ≦2.0 pg/mL/month are 0.21 times the odds of clinical recurrence in men with a pGS <7.

### Example 13

### A Prognostic Test for Identifying Men at a Reduced Risk for Prostate Cancer Recurrence Following Radical Prostatectomy

Methods used to estimate the risk of PSA-only recurrence post-prostatectomy (e.g., multivariate algorithms or nomograms) take into account several factors, but rely heavily on pathologic findings, such as Gleason score, surgical margin involvement, extracapsular extension, seminal vesicle invasion and lymph node involvement. However, these models cannot predict the risk of clinically documented prostate cancer recurrence (identified by biopsies, X-rays or scans) because of the heterogeneous natural history of prostate cancer progression after biochemical recurrence occurs (Shariat S F, Kattan M W, Vickers A J, et al. Future Oncology 2009; 5: 1555-1584; Moreira D M, Presti J C, Aronson W J, et al. J Urol 182:2250-2256, 2009.; D'Amico A V, Moul J W, Carroll P R, et al. J Natl Cancer Inst 95:1376-1383, 2003; Amling C L, Bergstralh E J, Blute M L, et al. J Urol 165:1146-1151, 2001; Stephenson, A. J., Kattan, M. W., Eastham, J. A., et al. J. Clin. Oncol. 24: 3973-3978, 2006; Shinghal, R., Yemoto, C., McNeal, J. E., Brooks, J. D., Urology 61:380-385, 2003.)

Following treatment e.g. radical prostatectomy, 1 in 5 men will recur with higher rates of recurrence, 40-60%, among men with adverse pathological risk factors (Han, M., Partin, A. W., Pound, C. R., et al. Urol. Clin. North Am. 28:555-565, 2001. Swanson, G. P., Riggs, M., Hermans, M. Urol. Oncol. 25:110-114, 2007). Men believed to be at a high risk recurrence because of adverse pathology often receive additional treatment with a goal of eliminating possible residual cancer. Empiric use of adjuvant radiation therapy (RT) in men with high-risk pathology clearly leads to overtreatment. It is estimated that 5 men would need adjuvant RT to prevent one from developing metastatic disease and 12 would need to be treated to prevent one cancer-related death (Thompson, I. M., Tangen, C. M., Paradelo, J., et al. J. Urol. 181:956-962, 2009.)

Accordingly, the instant disclosure is directed to a surprising and unexpected discovery of the improved efficacy and sensitivity in the predictive power of an extraordinarily sensitive PSA assay measured by a re-engineered version of immuno-PCR we called Nucleic Acid Detection Immunoassay. The assay provides the basis for a recently FDA cleared in-vitro diagnostic (IVD) test (http://www.accessdatalda.gov/cdrh docs/reviews/K101185.pdf) PSA RPI—which is indicated as an aid for identifying men at reduced risk of clinically recurrent prostate cancer in conjunction with established risk factors over an 8-year period post-RP. In the 510(k) study, a multivariate model that included pre-RP PSA, pathologic stage and Gleason score showed that PSA-RPI≦2.0 pg/mL/month (mo) was the most powerful independent prognostic factor for identifying men at reduced risk for clinical recurrence (HR 9.8, P<0.0001). While Gleason score (HR 5.4, P=0.0004) was also an independent predictor, PSA-RPI was the strongest independent prognostic index of recurrence risk, including the subset of men having pathologic Gleason score ≧7 prostate cancer.

The presence of positive surgical margins is considered a risk factor for recurrence and has been used to identify men as candidates for adjuvant RT post-RP. This study compared the prognostic strength of the PSA-RPI vs. surgical margin status to identify men at a reduced risk of post-RP clinical recurrence.

Study Details

The final selected study population consisted of 64 men with clinical recurrence and 240 controls (prevalence 21.1%). The linear slope of PSA in pg/mL vs. time post-RP in months (PSA-RPI) was calculated for each man via least squares linear regression. PSA-RPI slope was expressed as a binary categorical variable with the cutpoint set at 2.0 pg/mL/month for clinical investigation. A slope of ≦2.0 pg/mL/month was the pre-specified hypothesis for reduced risk of recurrence, therefore a slope >2.0 pg/mL/month denoted men “not at reduced risk for recurrence.”

The study was prospectively designed and incorporated archived serum samples from men treated by RP at four investigational sites (Duke University, Eastern Virginia Medical School, Memorial Sloan-Kettering Cancer Center, and the University of Washington) during 1990-2001. Institutional review boards at all sites approved the study protocol. Samples for analysis were de-identified such that no personally identifiable health information was released by the sites except for dates of birth, death, and clinical procedures, which were used to calculate age at RP, time to recurrence or length of follow-up. Inclusion criteria consisted of men with biopsy-confirmed prostate cancer treated by RP with a PSA time value in the first post-RP sample <0.1 ng/mL (<100 pg/mL) using standard-of-care immunoassays for PSA. Three serum samples obtained between 1.5 and 20 months post-RP were required with at least a two-month interval between each sampling time. Documentation of pre-RP PSA, pathologic stage and Gleason score was required to serve as covariates in multivariate analyses. Findings of positive surgical margins, extracapsular extension, seminal vesicle invasion, bladder neck invasion and positive lymph nodes were also recorded if documentation was available. Men categorized by the sites as non-recurring required a minimum post-RP follow-up of 8 years. Exclusion criteria were radiation treatment administered in the first 12 months post-RP, androgen deprivation therapy administered in the first 20 months post-RP, and serum samples stored >20 years.

Criteria for Response

Men categorized by the sites as recurrent required documented evidence of local recurrence by biopsy, evidence of metastases by imaging methods (MRI, CT, bone scan, or ¹¹¹In immunoscintigraphy in conjunction with CT), or death due to prostate cancer. Patients categorized as non-recurrent (no clinical evidence of disease) were followed after RP a median of 11.0 (IQR, 9.6-12.9) years versus a median of 4.7 (IQR, 2.7-8.4 years) for patients with clinically recurrent disease. Men with BCR without documented disease were categorized as non-recurrent.

Covariates in the multivariate model were pre-RP PSA, post-RP Gleason score and final pathologic stage—other risk factors were evaluated by univariate analysis.

Analytical Methods

Samples and 3 levels of PSA controls were assayed in duplicate in each run. 3 PSA calibrators (100, 25, 5 pg/mL) assayed in triplicate in each run. An AB 7500 Fast Dx PCR instrument was used for DNA amplification. Threshold cycles were converted to pg/mL using standard curve. Least-squares linear slope of PSA calculated in pg/mL/month.

Statistical Analysis

Categorical variables compared via the Pearson chi-square or Fisher's exact tests. Univariate Cox regression and Kaplan-Meier analysis were used to estimate the risk of recurrence for the PSA-RPI slope cutpoint and clinicopathologic factors. Multivariate Cox proportional hazards regression models were performed to determine the risk of recurrence of the PSA-RPI slope and clinical covariates.

Results

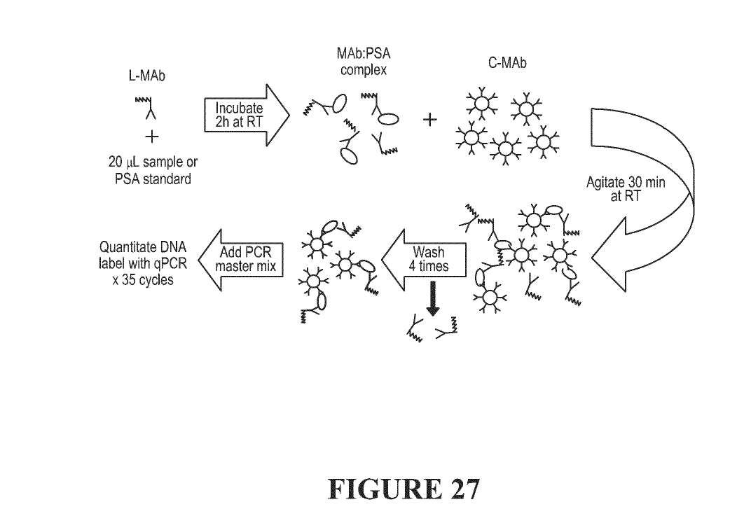

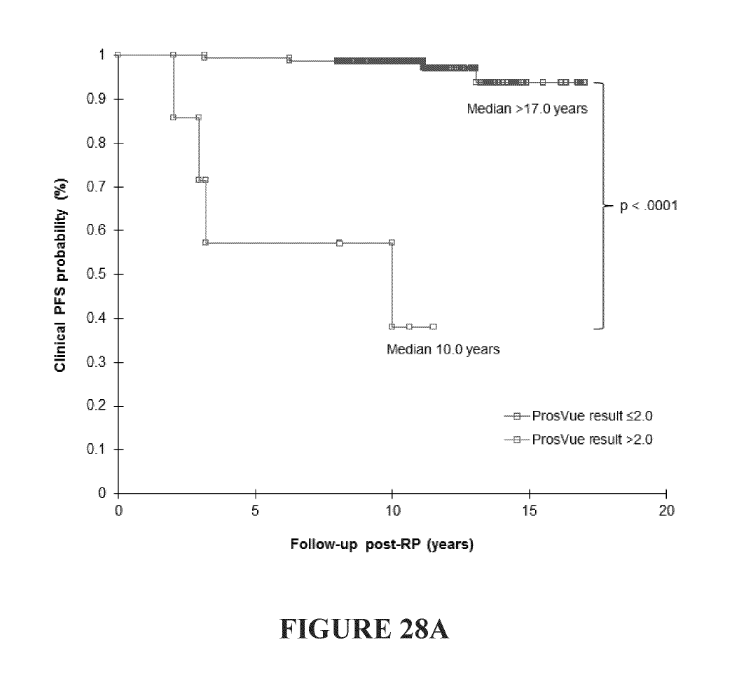

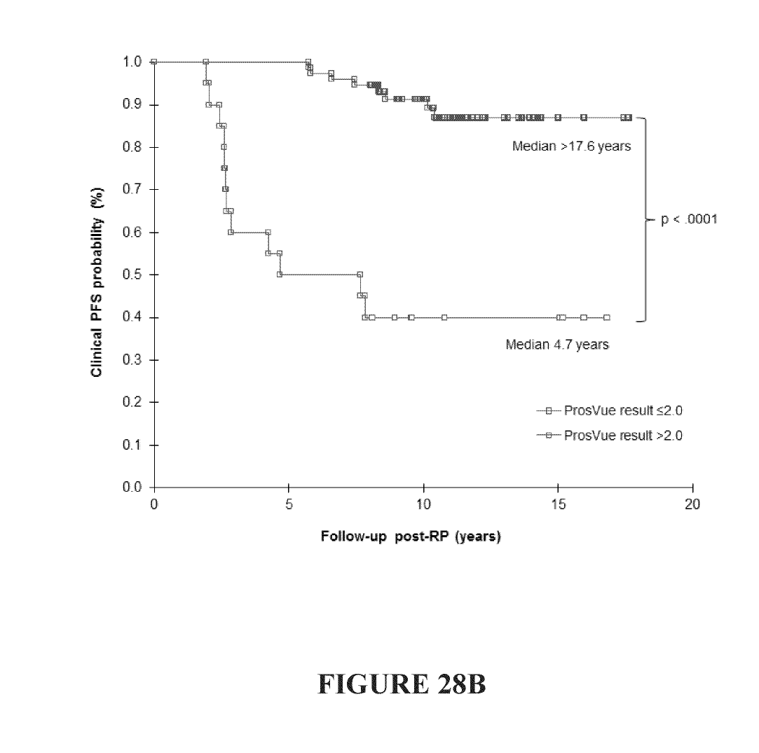

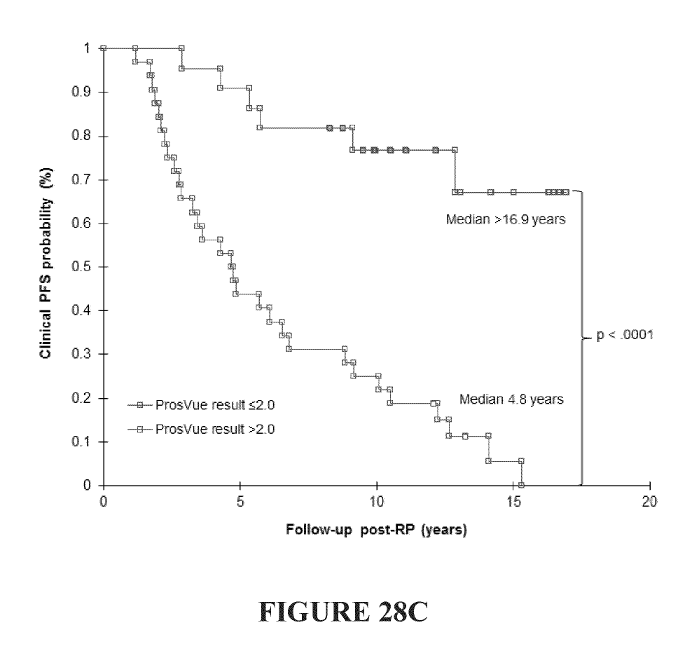

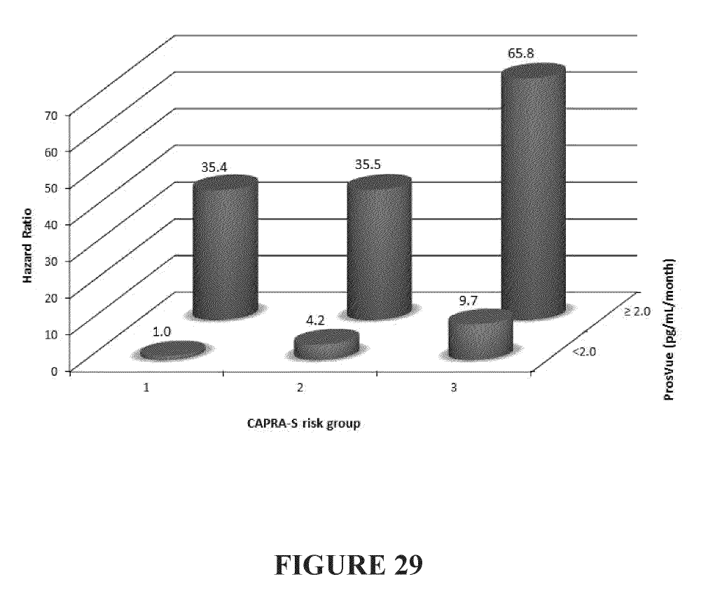

FIG. 27 describes the Assay Procedure. A PSA− slope ≦2.0 pg/mL/month was significantly associated with a reduced risk of recurrence by univariate Cox analysis (HR 18.3, 95% CI, 10.6-31.8, P<0.0001).

Covariates and most of the pathologic variables were also significantly associated with clinical recurrence using univariate analysis (Table 1).

In the multivariate model including pre-RP PSA, pathologic stage, and Gleason score the HR for PSA− slope was minimally attenuated to 9.8 (95% CI, 5.4-17.8, P<0.0001) and was an independent predictor of recurrence risk.

Median time to recurrence for men with PSA− slope ≦2.0 pg/mL/mo and those with negative margins exceeded 17.6 years (yrs) (FIG. 1).

The odds of recurrence when PSA− slope was ≦2.0 pg/mL/mo were not significantly difference whether margins were positive or negative (Table 2).

The odds of recurrence when PSA− slope was >2.0 pg/mL/month were not significantly different whether margins were negative or positive (Table 2).

TABLE 1
PSA-slope and clinical covariates

| | | | | |
|---|---|---|---|---|
| | | No. (%) | Univariate HR | |
| Variables | N | Recurred | (95% CI) | P value |

| | | | | | | | |
|---|---|---|---|---|---|---|---|
| PSA-slope | ≦2 pg/mL/month | 245 | 18 | (7.3) | 1.0 | | |
| | >2 pg/mL/month | 59 | 46 | (78.0) | 18.3 | (10.6-31.8) | <0.0001 |
| Pre-RP PSA | Continuous | 304 | 64 | (21.1) | 1.1 | (1.03-1.08) | <0.0001 |
| Gleason score | 4, 5 and 6 | 140 | 5 | (3.6) | 1.0 | | |
| | 7, 8 and 9 | 164 | 59 | (36.0) | 12.1 | (4.8-30.1) | <0.0001 |
| Pathologic stage | pT0-pT2 | 170 | 13 | (7.6) | 1.0 | | |
| | pT3-pT4 | 134 | 51 | (38.1) | 5.8 | (3.2-10.7) | <0.0001 |
| Surgical margins | Negative | 209 | 17 | (12.9) | 1.0 | | |
| | Positive | 95 | 37 | (38.9) | 3.3 | (2.0-5.4) | <0.0001 |
| Bladder neck | Absent | 146 | 24 | (16.4) | 1.0 | | |
| invasion | Present | 18 | 7 | (38.9) | 2.8 | (1.2-6.5) | 0.016 |
| % tumor volume | Continuous | 65 | 16 | (24.6) | 1.0 | (1.0-1.1) | 0.059 |

TABLE 2
Odds of clinical recurrence based on PSA-slope and surgical margin
status

| | Margin | | No. (%) | | Odds ratio | | |
|---|---|---|---|---|---|---|---|
| PSA-slope | status | N | Recurred | | (95% CI) | | P value |
| ≦2 pg/mL/ | Negative | 188 | 11 | (5.9) | 1.0 | | |
| month | Positive | 57 | 7 | (12.3) | 2.3 | (0.8-6.1) | 0.1108 |
| >2 pg/mL/ | Negative | 21 | 16 | (76.2) | 1.0 | | |
| month | Positive | 38 | 30 | (78.9) | 1.2 | (0.3-4.2) | 0.2440 |

CONCLUSIONS

PSA-slope ≦2.0 pg/mL/mo was found to be a stronger risk identifier for clinical recurrence of prostate cancer in men following RP than surgical margin status.

The description of specific embodiments of the invention described herein are not intended to be limiting or exclusive of other embodiments falling within the scope of the invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A method of determining the combined risk of prostate cancer recurrence in a subject following treatment and a post-treatment level of PSA of <100 pg/mL, comprising:
a) determining at least one pathological finding (PF) in the subject following treatment wherein the pathological finding is selected from:
i. a Gleason score of ≧7,
ii. a positive surgical margin,
iii. seminal vesicle invasion,
iv. extracapsular extension (ECE);
b) obtaining measurement of the PSA levels in two or more samples obtained from the patient within 18 months after treatment for prostate cancer,
wherein the PSA assay for measuring the PSA levels has a functional sensitivity less than 2.0 pg/mL, and the PSA levels from two or more samples are used to determine a [PSA]-time value, wherein the [PSA] time value does not exceed a PSA recurrence probability index and the combined recurrence risk of the subject is classified as PF+/PSA−; and
c) whereupon adjuvant treatment is not administered based on the [PSA]-time value and PSA recurrence probability index (RPI).

2. A method of determining the combined risk of prostate cancer recurrence in a subject following treatment and a post-treatment level of PSA of <100 pg/mL, comprising:
a) determining at least one pathological finding (PF) in the subject following treatment wherein the pathological finding is selected from:
i. a Gleason score of ≧7,
ii. a positive surgical margin,
iii. seminal vesicle invasion,
iv. extracapsular extension (ECE);
b) obtaining measurement of the PSA levels in two or more samples obtained from the patient within 18 months after treatment for prostate cancer,
wherein the PSA assay for measuring the PSA levels has a functional sensitivity less than 2.0 pg/mL, and the PSA levels from two or more samples are used to determine a [PSA]-time value, wherein the [PSA]-time value exceeds a PSA-RPI and the combined recurrence risk of the subject is classified as PF+/PSA+; and
c) whereupon adjuvant treatment is administered based on the combined risk classification using the [PSA]-time value and PSA-RPI in combination with the pathological findings.

3. A method of determining the combined risk of prostate cancer recurrence in a subject following treatment and a post-treatment level of PSA of <100 pg/mL, comprising:
a) determining at least one pathological finding (PF) in the subject following treatment wherein the pathological finding is selected from:
i. a Gleason score of <7,
ii. a negative surgical margin,
iii. a negative seminal vesicle invasion,
iv. a negative extracapsular extension (ECE);
b) obtaining measurement of the PSA levels in two or more samples obtained from the patient within 18 months after treatment for prostate cancer,
wherein the PSA assay for measuring the PSA levels has a functional sensitivity less than 2.0 pg/mL, and the PSA levels from the two or more samples are used to determine a [PSA]-time value, wherein the [PSA] time value does not exceed a PSA RPI and the combined recurrent risk of the subject is classified as PF−/PSA−; and
c) whereupon adjuvant treatment is not administered based on the combined risk classification using the [PSA]-time value and PSA-RPI in combination with the pathological findings.

4. A method of determining the combined risk of prostate cancer recurrence in a subject following treatment and a post-treatment level of PSA of <100 pg/mL, comprising:
a) determining at least one pathological finding (PF) in the subject following treatment wherein the pathological finding is selected from:
i. a Gleason score of less than 7,
ii. a negative surgical margin,
iii. a negative seminal vesicle invasion,
iv. a negative extracapsular extension (ECE);
b) obtaining measurement of the PSA levels in two or more samples obtained from the patient within 18 months after treatment for prostate cancer,
wherein the PSA assay for measuring the PSA levels has a functional sensitivity less than 2.0 pg/mL, and the PSA levels from the two or more samples are used to determine a [PSA]-time value, wherein the [PSA]-time value exceeds a PSA RPI of 2.0 pg/mL/month and the combined recurrent risk of the subject is classified as PF−/PSA+; and
c) whereupon adjuvant treatment is administered based on the classification using the [PSA]-time value and PSA-RPI.

5. The method of claim 2 or 4 wherein the likelihood of recurrence is greater than for the subject who is classified as PSA+ than for a subject who is classified as PSA/− and PF+.

6. The method of claim 2 or 4 wherein the PF is seminal vesicle invasion.

7. The method of claim 6 wherein the likelihood of recurrence is greater than for the subject who is classified as PSA+ than the subject who is classified as PSA/− and seminal vescicle positive.

8. The method of claim 6 wherein the likelihood of recurrence is greater than for the subject who is classified as PSA+ than the subject who is classified as PSA/− and seminal vescicle negative.

10. The method of claim 2 or 4 wherein the PF is ECE.

11. The method of claim 10 wherein the likelihood of recurrence is greater than for the subject who is classified as PSA+ than the subject who is classified as PSA/− and ECE positive.

12. The method of claim 10 wherein the likelihood of recurrence is greater than for the subject who is classified as PSA+ than the subject who is classified as PSA/− and ECE negative.

13. The method of claim 2 or 4 wherein the PF is surgical margins.

14. The method of claim 13 wherein the likelihood of recurrence is greater than for the subject who is classified as PSA+ than the subject who is classified as PSA/− and surgical margins positive.

15. The method of claim 13 wherein likelihood of recurrence is greater than for the subject who is classified as PSA+ than the subject who is classified as PSA/− and surgical margins negative.

16. The method of claim 2 or 4 wherein the PF is Gleason score.

17. The method of claim 16 wherein the likelihood of recurrence is greater than for the subject who is classified as PSA+ than the subject who is classified as PSA/− and a Gleason score of more than 7.

18. The method of claim 16 wherein the likelihood of recurrence is greater than for the subject who is classified as PSA+ than the subject who is classified as PSA/− and a Gleason score of less than 7.
